(19) United States
(12) Patent Application Publication
(10) Pub. No.: US 2023/0257804 A1
(43) Pub. Date: Aug. 17, 2023

(54) SWITCH OLIGONUCLEOTIDE

(71) Applicant: Oxford University Innovation Limited, Oxford (GB)

(72) Inventors: Wei HUANG, Oxford (Oxfordshire) (GB); Zhanfeng CUI, Oxford (Oxfordshire) (GB); Boon Chuan LIM, Oxford (Oxfordshire) (GB); Chia-Chen HSU, New Taipei City (Taiwan) (TW); Yejiong YU, Oxford (Oxfordshire) (GB)

(21) Appl. No.: 18/020,700

(22) PCT Filed: Aug. 10, 2021
(86) PCT No.: PCT/GB2021/052059

§ 371 Date: Feb. 10, 2023

(30) Foreign Application Priority Data
Aug. 11, 2020 (GB) 2012480.6

(51) Int. Cl. C12Q 1/6853; C12Q 1/70

(52) U.S. Cl. CPC C12Q 1/6853; C12Q 1/701; C12Q 2600/16

(57) ABSTRACT

The invention relates to a set of oligonucleotide primers and their use in methods for amplifying and detecting oligonucleotides, detecting pathogens, or diagnosing infections such as SARS-CoV-2 and Covid-19. The primer set includes a switch oligonucleotide that is adapted to anneal to a forward or reverse primer of the set at temperatures that are below the temperature range for amplification of the DNA. The switch oligonucleotide prevents amplification from the complementary primer when the complementary primer is bound to the switch oligonucleotide. The invention also provides a method of reducing false positives in the detection of a target DNA or RNA sequence using loop-mediated isothermal amplification (LAMP) or reverse transcription loop-mediated isothermal amplification (RT-LAMP), by using such a switch oligonucleotide.

### FIELD OF THE INVENTION

The disclosure relates to a set of oligonucleotide primers and their use in methods for amplifying and detecting oligonucleotide, detecting pathogens, or diagnosing infections such as SARS-CoV-2 and Covid-19.

### BACKGROUND TO THE INVENTION

Loop-mediated isothermal amplification (LAMP) is a rapid technology of DNA amplification (Notomi et al. 2000, Tomita et al. 2008). which has been applied to pathogen detection such as virus, bacteria and malaria. The LAMP reaction generally takes place in a constant temperature, and the target DNA can be amplified in 30 min. The LAMP method employs 4 or 6 primers to bind six regions of a target DNA and the specificity is extremely high. Since the LAMP only needs one constant temperature, the device can be simple. Initially LAMP used 4 primers, but later it was found that the inclusion of two additional loop primers could shorten the time required for the original LAMP and potentially increase sensitivity (Nagamine et al. 2002). The availability of WarmStart RTx Reverse Transcriptase (New England Biolabs, UK) makes it possible to combine both reverse transcription and LAMP in one reaction (RT-LAMP).

The inventors have previously developed a rapid COVID-19 test kit for the detection of SARS-CoV-2, using one step RT-LAMP without RNA extraction (Huang et al. 2020). The whole reaction can be as short as 20 minutes at a constant 65° C. A simple colour-change indication can be visualised by the naked eye to confirm the result of viral RNA amplification.

The use of four to six primers, recognising six distinct target sequences provides for a high degree of specificity and selectivity in LAMP methods, which is particularly important in diagnostic applications. Nonetheless, several reports have indicated that carryover contamination, the formation of primer-dimers and self-amplification can result in false positives when conducting RT-LAMP assays (Baek et al. (2020); Dao et al. (2020); Hsieh et al. (2014)).

### SUMMARY OF THE INVENTION

The present inventors have developed a method of LAMP or RT-LAMP with improved stability and reliability. The method uses a short oligonucleotide designed as a switch, which comprises a sequence that is complementary to a sequence used by one of the LAMP reaction primers. The oligonucleotide serves as a temperature-dependent switch to bind an essential primer of LAMP. When the temperature is lower than working temperature of LAMP (e.g. 65° C.), the switch binds the complementary primer and inhibits unspecific or off-target amplification. At the working temperature, the switch dissociates from the primer, allowing it to bind target RNA/DNA for reverse transcription and amplification of nucleic acids.

The inventors have demonstrated the efficiency of the oligonucleotide switch in improving the stability and reliability of (RT-)LAMP and reducing the incidence of false positives in detecting a viral RNA in a sample using a previously described RT-LAMP method for detecting SARS-CoV-2 (Huang et al. 2020). The inventors have further developed a vacuum-dried master mix for single step RT-LAMP reaction, simplifying the operation for end users and easing long-term storage and transportation. The new RT-LAMP assay has been applied for testing RNA samples extracted from 72 patients, demonstrating an overall percentage agreement of 90% relative to RT-qPCR. The outcome of RT-LAMP can be reported by both colorimetric and fluorescent reading, which can be decided by the naked eye, and simultaneously quantified by fluorescent intensity. Such dual-display can help data interpretation and clinical diagnosis. The inventors have also validated the application of RT-LAMP to direct detection of SARS-CoV-2 in 47 clinical oro-nasopharyngeal swab samples without any pre-treatment and RNA extraction, with an overall percentage agreement of 89%. This improved, inexpensive and rapid colorimetric assay has considerable practical benefits over the current standard RT-qPCR assays and has great potential to be deployed as a first-line screening tool.

Accordingly, in a first aspect, the invention provides a set of oligonucleotide primers for use in a method of amplifying a fragment of DNA, the set of primers comprising a forward inner primer (FIP), a reverse inner primer (BIP), a forward outer primer (F3), a reverse outer primer (B3), and a switch oligonucleotide, wherein the switch oligonucleotide comprises a nucleotide sequence that is complementary to a fragment of one of the forward or reverse primers, wherein the switch oligonucleotide is adapted to anneal to the complementary primer at temperatures that are below the temperature range for amplification of the DNA, and wherein the switch oligonucleotide prevents amplification from the complementary primer when the complementary primer is bound to the switch oligonucleotide.

In a further aspect, the invention provides a method of reducing false positives in the detection of a target DNA or RNA sequence using loop-mediated isothermal amplification (LAMP) or reverse transcription loop-mediated isothermal amplification (RT-LAMP), wherein the method comprises including a switch oligonucleotide in the LAMP or RT-LAMP reaction, wherein the switch oligonucleotide comprises a nucleotide sequence that is complementary to a fragment of a forward inner (FIP), reverse inner (BIP), forward outer (F3) or reverse outer (B3) primer used for the amplification, wherein the switch oligonucleotide is adapted to anneal to the complementary primer at temperatures that are below the temperature range for the amplification, and wherein the switch oligonucleotide prevents amplification from the complementary primer when the complementary primer is bound to the switch oligonucleotide.

In a further aspect, the invention provides a method of loop-mediated isothermal amplification (LAMP) or reverse transcription loop-mediated isothermal amplification (RT-LAMP), the method comprising (a) mixing the set of primers of claim 1 with template DNA or RNA, deoxyribonucleotide triphosphates (dNTP), DNA polymerase and optionally reverse transcriptase in solution; and (b) heating the mixture to the working temperature of the DNA polymerase

In some cases the switch oligonucleotide is adapted to prevent elongation at the 3′ end of the switch oligonucleotide and/or comprises a dark quencher moiety.

In a further aspect, the invention provides a kit for amplifying a fragment of DNA, wherein the kit comprises the set of primers (including the switch oligonucleotide). In some cases, the kit comprises further components, such as (i) DNA polymerase; (ii) reverse transcriptase; (iii) a pH indicator and/or colorimetric indicator; (iv) a fluorophore; (v) deoxyribonucleotide triphosphates (dNTP); (vi) buffer components; and/or (vii) instructions for use. In some cases the set of primers and optionally one or more of the additional components of the kit are dried and may be combined as a reagent mix.

In a further aspect, the invention provides an the use of the set of primers or the kit in a method of detecting or amplifying a target DNA or RNA sequence.

In some cases, the method comprises reverse transcription of an RNA to produce cDNA and amplification of the reverse transcribed cDNA.

In some cases, the method is for detecting a polynucleotide of a pathogen in a sample, such as a virus, such as a Coronaviridae or SARS-CoV-2.

In some cases the complementary primer comprises the nucleotide sequence of SEQ ID NO: 1 or SEQ ID NO: 2, or a variant having at least 50% sequence identity to SEQ ID NO: 1 or SEQ ID NO: 2, and/or wherein the switch oligonucleotide comprises the nucleotide sequence of SEQ ID NO: 8 or a variant having at least 50% sequence identity to SEQ ID NO: 8. the set of primers further includes a reverse inner primer (BIP) comprising the nucleotide sequence of SEQ ID NO: 3 or a variant having at least 50% sequence identity to SEQ ID NO: 3; a forward outer primer (F3) comprising the nucleotide sequence of SEQ ID NO: 4 or a variant having at least 50% sequence identity to SEQ ID NO: 4; a reverse outer primer (B3) comprising the nucleotide sequence of SEQ ID NO: 5 or a variant having at least 50% sequence identity to SEQ ID NO: 5; a forward loop primer (LF) comprising the nucleotide sequence of SEQ ID NO: 6 or a variant having at least 50% sequence identity to SEQ ID NO: 6; and/or a reverse loop primer (BF) comprising the nucleotide sequence of SEQ ID NO: 7 or a variant having at least 50% sequence identity to SEQ ID NO: 7. In some cases the set of primers comprise six further primers having the nucleotide sequences of SEQ ID NOS: 9 to 14.

In a further aspect, the invention provides a kit for detecting SARS-CoV-2 or for diagnosing a SARS-CoV-2 infection or Covid-19 in a subject, the kit comprising a set of six primers and a switch oligonucleotide having the nucleotide sequences of SEQ ID NOs: 2 to 8 or a set of 12 primers and a switch oligonucleotide having the nucleotide sequences of SEQ ID NOs: 2 to 14, a DNA polymerase, a reverse transcriptase, a colorimetric pH indicator, deoxyribonucleotide triphosphates (dNTP), and optionally a buffer, optionally wherein the kit comprises a vacuum-dried reagent mix.

In a further aspect, the invention provides a method of detecting SARS-CoV-2 in a sample or diagnosing a SARS-CoV-2 infection or COVID-19 in a subject, the method comprising (i) obtaining the sample, or obtaining a biological sample from the subject; (ii) reverse transcription to produce cDNA from RNA in the sample; (iii) amplification of the reverse transcribed cDNA using a set of six primers and a switch oligonucleotide having the nucleotide sequences of SEQ ID NOs: 2 to 8 or a set of 12 primers and a switch oligonucleotide having the nucleotide sequences of SEQ ID NOs: 2 to 14; (iv) detecting the amplified DNA; and determining the presence of SARS-CoV-2 or diagnosing a SARS-CoV-2 infection or COVID-19 in the subject.

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a primer” generally includes two or more such primers.

Section headings are used herein for convenience only and are not to be construed as limiting in any way.

Unless otherwise defined, all technical and scientific terms used in this application have the same meaning as commonly understood by those skilled in the technical field of this application. The terminology used in the description of this application is only for the purpose of describing specific embodiments, and is not used to limit this application. The term “and/or” as used in this application includes any and all combinations of one or more related listed items. The experimental methods without specific conditions in the following examples generally follow conventional conditions or the conditions recommended by the manufacturer. The various commonly used chemical reagents used in the examples are all commercially available products.

### DESCRIPTION OF THE FIGURES

FIG. 1 Stability test of improved RT-LAMP reaction mix. a, Stability test of RT-LAMP reaction mix containing O117_N and O117_Q. Tubes were added with Synthetic RNA Control 2—MN908947.3 (‘+’) or human genome cDNA (‘−’) followed by heating at 65° C. for the time indicated by T. A, B, and C represent three technical replicates. b(i) Dried reaction mixes stored at room temperature up to 3 days. (ii) Dried reaction mixes reconstituted with 20 μL DNase/RNase free water followed by the addition of Synthetic RNA Control 2—MN908947.3 (‘+’) or human genome cDNA (‘−’). (iii) Tubes incubated at 65° C. for 30 minutes. Top row: Dried reaction mixes containing O117_N; Bottom row: Dried reaction mixes containing O117_Q.

FIG. 2—The switch can stabilise RT-LAMP performance in unstable heating device. In a stable heating device, O117_N and O117_Q performed well in 1 hour. In an unstable heating device, O117_N generated false positive results due to self- and off-target amplification in human RNA control sample 2. O117_Q still shows negative to human RNA control.

FIG. 3—RT-LAMP performance of pH corrected decolored kits that were stored at room temperature for 7 days. Colour change of dried to yellow after storage of 7 days (a). The pH of kits can be adjusted by adding KOH and the colour of the adjusted mixes (b) produced a similar colour to original fresh mixes (c). The pH colour-corrected mixes can perform colorimetric detection of Synthetic RNA Control 2—MN908947.3 (Twist Bioscience) (d and e). 1 and 2: pH adjusted mixes with viral RNA, 3. Blank control of pH adjusted mix with water. 4 and 5: fresh mixes with viral RNA. 6 and 7: Blank control of fresh mixes with water. (f) Fluorescent detection of the amplification process of pH corrected and fresh mixes.

FIG. 4—Long-term storage of optimized RT-LAMP dried reaction mixes at room temperature. Dried mixes were stored for a, 7 days and b, 14 days at room temperature. (i) Dried mixes were (ii) reconstituted with 20 μL DNase/RNase free water followed by the addition of 5 μL of Synthetic RNA Control 2—MN908947.3 (Twist Ltd) (sample 1, 2, 3) or human genome cDNA (sample 4, 5). (iii) Tubes were incubated at 65° C. for 30 minutes. c, Real-time fluorescence curve showing the amplification of dried reaction mixes stored for 14 days at room temperature. 0.5 μL of fluorescent dye (New England Biolabs) was added to the tubes in b(ii) and incubated at 65° C. in a qPCR machine. Each cycle represents 30 seconds and the reaction was run for 30 min.

FIG. 5—Sensitivity analysis of improved RT-LAMP assay. SARS-CoV-2 RNA transcript and reaction mix used in each experiment are labelled on the top right white box of each image. ‘+’ indicates a positive result while ‘−’ indicates a negative result. A, B, C, D, E represents five technical replicates.

FIG. 6—Sensitivity and specificity of improved RT-LAMP assay. a, Determination of 50% endpoints for O117_Q and O117_N preparations of RT-LAMP assay. Full length transcripts were serially diluted in buffer AVE to achieve the total RNA input/reaction indicated on the y axis. Each dot represents one experimental replicate. Dashed lines indicate 50% endpoint as calculated by the Reed-Muench method. Kits were dried except where indicated for RNA Control 2. Full length transcript indicated on figure. Dried reaction mix containing b, O117_N and c, O117_Q were tested against non-SARS human-infective coronaviruses. A and B represent two technical replicates.

FIG. 7—Detection of SARS-CoV-2 from clinical RNA extracts by RT-LAMP. Performance of RT-LAMP assay using wet reaction mix containing O117_Q on detecting SARS-CoV-2 from clinical RNA extract samples. Data was collected on 22 May 2020. A1-A10 and H1-H8 are the negative controls, H11 and H12 are the positive controls.

FIG. 8—Quantitative evaluation of pH-dependent colorimetric RT-LAMP readout. Three quantitative methods a, Absorbance using 430/560 nm ratio; b, Syto-9 fluorescence using 485 nm (excitation) and 500 nm (emission); and c, Qubit fluorescence using Qubit 2.0 fluorometer were used to assess the RT-LAMP result. d, e, and f are the correlation analysis between RT-qPCR and the three quantitative evaluations, respectively. Each dot represents one experimental replicate. Dotted lines indicate 3× standard deviations above the negative controls.

FIG. 9—Detection of SARS-CoV-2 from oro-nasopharyngeal swab specimen. Performance of RT-LAMP assay using dried reaction mix containing O117_Q in detecting SARS-CoV-2 from unprocessed and unextracted oro-nasopharyngeal swab sample. Data was collected on a, 1 May 2020; b, 22 May 2020; c, 3 Jun. 2020 and d, 11 Jun. 2020. ‘+’ indicates positive control while ‘−’ indicates negative control. e, Comparison of RT-LAMP for detecting SARS-CoV-2 RNA in extract and directly from swab versus RT-qCR Ct value.

FIG. 10—Two sets of primers (O117_Q and S17) in one reaction enhanced detection sensitivity of SARS-CoV-2 virus in RT-LAMP assay. Two sets of primers with the switch showed enhanced sensitivity and no false positive results, whilst those without the switch resulted in false positive results.

FIG. 11—The switch can stabilise RT-LAMP performance within an hour of incubation. O117_N generated 60% false positive results (12/20 reactions) due to self- and off-target amplification in human RNA control sample after incubating at 65° C. for 60 minutes. O117_Q still shows negative to human RNA control as long as 60 minutes at 65° C. reaction.

FIG. 12—Long-term storage of optimized RT-LAMP dried reaction mixes at room temperature. Dried mixes were stored for day 0 (A) and day 15 (B) at room temperature. Dried mixes were reconstituted with 20 μL DNase/RNase free water followed by the addition of 5 μL of Synthetic RNA Control 2—MN908947.3 (40 copies/μL, Twist Bioscience) or human genome DNA as negative control. (iii) Tubes were incubated at 65° C. for 30 minutes. Each time three batches and each batch three replicates were carried out.

FIG. 13—Detection of SARS-CoV-2 from 444 negative RNA extracts by RT-LAMP. Performance of RT-LAMP assay using freeze drying reaction mix containing O117_Q on detecting SARS-CoV-2 from RNA extract from saliva samples. PC: positive control and NC: negative control.

FIG. 14—Detection of SARS-CoV-2 from 444 negative RNA extracts by RT-LAMP. Performance of RT-LAMP assay using freeze drying reaction mix containing O117_Q on detecting SARS-CoV-2 from RNA extract from saliva samples. PC: positive control and NC: negative control.

FIG. 15—Detection of SARS-CoV-2 from RNA extracts from swab and saliva specimen. Comparison of RT-LAMP for detecting SARS-CoV-2 RNA in extract vs RT-qPCR Ct value. RT-LAMP results were read by colorimetric changes (see FIG. 7, 13, 14).

### DETAILED DESCRIPTION OF THE INVENTION

### Primers

In some aspects the invention relates to a set of primers. The primers are suitable for use in a method of LAMP and/or RT-LAMP. The primers are short, often chemically synthesized, polynucleotides, typically oligonucleotide primers. The primers typically have a free 3′-hydroxyl moiety on the terminal sugar.

Nucleic acid polymerases generally requires a free 3′-hydroxyl moiety on the terminal sugar of a stretch of double stranded nucleic acid adjacent to the site of new synthesis. The primer is able to anneal to a template nucleic acid, typically at a site having a complementary sequence, to provide an initiation site for the polymerase synthesis reaction. In some cases a 3′ modification, such as a sulfydryl, may be utilized to prime the synthesis reaction.

The primer is targeted to complementary sequences by virtue of its specific base-pairing capacity. Formation of hybrids between the primer and target nucleic acid are typically formed by incubation of the two in solution under conditions of salt, pH, and temperature that allow spontaneous annealing.

The primers used are typically DNA. The primers may in some cases be PNA or RNA. The primers may comprise any combination of natural or canonical nucleotides (i.e., “naturally occurring” or “natural” nucleotides), which include adenosine, guanosine, cytidine, thymidine and uridine. The primers may also comprise nucleotide analogues. For example, the primers may include one or more peptide nucleotides, in which the phosphate linkage found in DNA and RNA is replaced by a peptide-like, N-(2-aminoethyl)glycine. Peptide nucleotides undergo normal Watson-Crick base pairing and hybridize to complementary DNA/RNA with higher affinity and specificity and lower salt-dependency than normal DNA/RNA oligonucleotides and may have increased stability. The primers may include one or more locked nucleotides (LNA), which comprise a 2′-O-4′-C-methylene bridge and are conformationally restricted. LNA form stable hybrid duplexes with DNA and RNA with increased stability and higher hybrid duplex melting temperatures. The primers may include one or more Propynyl dU (also known as pdU-CE Phosphoramidite, or 5′-Dimethoxytrityl-5-(1-Propynyl)-2′-deoxyUridine,3′-[(2-cyanoethyl)-(N,N-diisopropyl)]-phosphoramidite). The primers may include one or more unlocked nucleotides (UNA), which are analogues of ribonucleotides in which the C2′-C3′ bond has been cleaved. UNA form hybrid duplexes with DNA and RNA, but with decreased stability and lower hybrid duplex melting temperatures. LNA and UNA may therefore be used to finely adjust the thermodynamic properties the primers in which they are incorporated. The primers may include one or more triazole-linked DNA oligonucleotides, in which one or more of the natural phosphate backbone linkages are replaced with triazole linkages, particularly when click chemistry is used for synthesising the primers. The primers may include one or more 2′-O-methoxy-ethyl bases (2′-MOE), such as 2-Methoxyethoxy A, 2-Methoxyethoxy MeC, 2-Methoxyethoxy G and/or 2-Methoxyethoxy T. The primers may include one or more 2′-O-Methyl RNA bases. The primers may include one or more 2′-fluoro bases, such as fluoro C, fluoro U, fluoro A, and/or fluoro G. Other specific examples of nucleotide analogues include 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, 5-Methyl dC, 5-Nitroindole, 5-hydroxybutynl-2′-deoxyuridine (Super T) and 8-aza-7-deazaguanosine (Super G). In some cases the primers may include super T 2,6-Diaminopurine (2-Amino-dA) and/or 5-Methyl dC. The primers may include one or more biotinylated nucleotides. In some cases the primers may comprise up to 2%. 5%, 10%, 15%, 20%, 25%, 30%, 35%, 40% or more nucleotide analogues and/or biotinylated nucleotides, or any one type of nucleotide analogue as described herein. Use of synthetic oligonucleotides with a non-hydrolysable backbone at the ultimate and/or penultimate link may be beneficial to reduce reaction noise. Alternative backbones could be selected from the considerable range of chemistries available such as phosphorothiorate, morpholino, locked nucleic acid, or peptide nucleic acid.

Primers may be synthesized according to standard techniques. Modified bases and/or linker backbone chemistries may be desirable and functional in some cases. In some cases, the primer may be modified at their ends, either 5′ or 3′, with groups that serve various purposes e.g. fluorescent groups, quenchers, protecting (blocking) groups (reversible or not), magnetic tags, proteins, radioactive labels etc.

The primers typically have a high grade of purity, i.e. before being included in a reaction mix. For example HPLC purified primers, particularly the FIP and BIP primers as described herein, may be used.

LAMP methods typically use at least 4 primers that are selected or adapted to hybridise/anneal to 6 different sequences in the template nucleic acid.

Two inner primers, referred to herein as the forward inner primer (FIP) and the reverse (backward) inner primer (BIP), each contain two distinct sequences corresponding to the sense and antisense sequence of the template DNA. The 3′ end sequences of the forward inner primers, referred to as “F2” for the FIP and “B2” for the BIP, are adapted to anneal by hybridization to the template DNA at corresponding complementary sequence regions (referred to as F2c and B2c) and prime the initial polymerase step of LAMP amplification.

The annealing sites of F2 and B2 flank and define the ends of the region of the template DNA that is amplified. The amplified region of template DNA is typically up to about 1 kb, or 500 bp, 400 bp, 300 bp or 250 bp, or is between about 120 bp, or 130 bp, or 140 bp or 150 bp, or 160 bp and about 300 bp or 300 bp, or is between about 130 and 300 bp or between about 230 and 270 bp or between about 240 and 260 in length, including the B2/B2c and F2/F2c regions.

The 5′ end of the FIP and BIP primers, referred to as “FIc” and “BIc”, have high sequence identity to a sequence 5′ to F2c and B2c, respectively, i.e. the annealing site of F2 and B3, respectively, at the 3′ end of FIP and BIP, respectively. After initial strand extension from the FIP, the FIP/nascent strand is displaced from the template as described below, and F1c of the FIP and the complementary sequence of the nascent strand, F1, self-anneal to form a loop at one end of the strand.

Both ends of the inner primers are typically not AT-rich. In some cases the terminal three nucleotides is not an A or a T. In other cases, as least 3 out of the terminal 4 nucleotides, or 4 out of the terminal 5 nucleotides are not an A or a T″ ?

The distance between the 3′ end of F1 and the 3′ end of F2, and between the 3′ end of B1 and the 3′ end of B2 in the template DNA is typically about 30 to 70, or more typically about 40 to 60 nucleotides in length, or about 40 nucleotides in length.

Between the F2 and F1c in the FIP (and between B2 an B1c in the BIP) is a spacer region. F2 (or B2) and the spacer region (or their reverse complements) together form part of the loops that form at each end of the initial dumbbell-like structure formed during the initial stage of the LAMP reaction, and each loop added during the cyclic amplification stage. This loop is formed when the 5′ end of the primer self-anneals to new complementary strand primed at the 3′ end of the primer, using the template sequence with high sequence identity to the 5′ end of the primer as template. The rest of the loop is complementary to the region between F1 and F2 (or F1c and F2c, or B1 and B2 or B1c and B2c). This additional loop sequence provides the template for binding by a loop primer in some cases, as described below.

The spacer/loop region is typically at least 2, or at least 3 or 4 nucleotides in length. The spacer/loop region is typically less than about 50, or less than about 40, or 30 or 10 nucleotides in length. The spacer/loop region may in some cases comprise or consist of a polythymidine. In other cases the spacer/loop region may have the sequence of the FIP primer exemplified in the Examples herein.

The outer forward (“F3”) and reverse (“B3”) primers are adapted to anneal by hybridization to a single stretch of template DNA, typically across substantially the whole length of the primer. They prime a polymerase reaction that displaces the FIP or BIP primers and nascent strand formed at the 3′ end of the inner primers using the template DNA in the initial polymerase reactions of the LAMP method. Hence, the F3 primer anneals to a sequence of the template that is 3′ to F2c, i.e. the sequence hybridized by the F2 sequence of the FIP primer. The B3 primer anneals to a sequence of the template that is 3′ to B2c, i.e. the sequence hybridized by the B2 sequence of the BIP primer. The distance between the sequence hybridized by the forward inner and outer primers or the reverse inner and outer primers is typically about 5 to 100 nucleotides, for example about 10 to 50 nucleotides.

The primary purpose of the outer primers in displacing the inner primers and nascent strand from the template nucleic acid is to form the dumbbell-like structure that is used as initial template for the cycling amplification step of LAMP. This cycling step uses further inner forward and reverse primers to prime the cyclic amplification, whilst the outer primers typically perform no further role. Hence, in a typical reaction, the inner forward and reverse primers are present in excess of the outer forward and reverse primers, typically by about 2× to 10×, or about 3× to 5×, or about 4×.

In some cases an additional set of forward and/or reverse primers is included. Nagamine et al. (2002) described how the inclusion of two additional loop primers (the “LF” and “LB” primers) could shorten the time required for the original LAMP and potentially increase sensitivity. The loop primers hybridize to the loop regions of the dumbbell-like structure described above, except for the part corresponding to or hybridizing to the sequence of the inner primers.

Typically, the LF primer anneals by hybridization to the region of the loop between the F1 and F2 regions. Alternatively the, the LF primer may anneal by hybridization to the region of the loop between the F1c and F2c regions. Typically, the LB primer anneals by hybridization to the region of the loop between the B1 and B2 regions. Alternatively the, the LB primer may anneal by hybridization to the region of the loop between the B1c and B2c regions.

The forward and loop primers are typically in about the same quantity as the forward and reverse inner primers, in some cases +/−50%, 40%, 30%, 20%, 10%, 5%, 2%, or 1%.

Each of the F1c, F2, F3, B1c, B2 and B3 regions, which anneal by hybridization to corresponding reverse complement regions of template DNA, is typically at least 10, or at least 11, 12, 13, 14, 15, 16, 17, 18, 19 or 20 nucleotides in length, such as from about 10 to about 60, about 12 to about 40, or about 15 to about 25 or 30 nucleotides in length. In some cases, the primer may comprise one or more nucleotide analogues, such as analogues described herein, that form double-stranded hybrids with higher stability than natural nucleotides. In this case, the primer could, in principal, be shorter, such as at least 6, 7, 8 or 9 nucleotides in length, for example. In general, however, shorter regions will have lower specificity and selectivity and may be more prone to off target amplification. Typically the inner primers are longer that the outer primers, because they include two separate template-binding regions (F2 and F1c; or B2 and B1c) and the spacer/loop region in-between. Accordingly, the inner primers typically have a length of about 24, or 30, or 35, or 40, to about 100, or 90, or 80, or 70, or 60, or 50 nucleotides, or about 30 to 70, or about 35 to 60, or about 40 to 50 nucleotides in length. The outer and loop primers are typically comprise their template-binding region across substantially their whole length, but may in some cases have additional nucleotides, such as 1-5, or 1-10, or 1-15, or 1-20 additional nucleotides at their 5′ end. These primers are typically between about 10 to about 50 nucleotides in length, for example, about 12 to about 40, or about 15 to about 25 or 30 nucleotides in length, or longer if they include additional nucleotides at their 5′ end as described above.

The choice of single stranded regions will depend, in part, on the complexity of the starting nucleic acid so that for example, a human genome may require a longer primer while a plasmid may require a much shorter primer.

It should be noted that amplification may in some cases be achieved even if the primers are not completely complementary to the template nucleic acid, as long as the primers are able to anneal to the target region of the template DNA. In general, however, primers will be designed to bind to conserved regions of a template DNA, and reaction conditions will be selected, to achieve maximum specificity, selectivity and efficiency of the amplification method.

The primers are typically selected to have a melting temperature (Tₘ) that is optimal for use in a particular DNA polymerase. Typical Tₘ values are about 50 to about 70° C., more typically about 55 to 68° C., more typically about 55 or 60 to 65° C. In some cases, the Tₘ values of the F1c and B1c regions of the inner primes are set slightly higher than those of the F2 and B2 regions, so that the loop is formed immediately after release of the single stranded DNA from template. In some cases, the Tₘ values of the outer primers (F3 and B3) regions are set lower than those of the F2 and B2 regions of the inner primers, to promote initial synthesis from the inner primers. In both cases, the difference in Tₘ values may independently be up to or about 2° C., 1.5° C., 1° C., 0.5° C., 0.2° C., or 0.1° C.

In some cases the primers of the invention may be used to amplify/detect nucleic acid associated with a pathogen. The primers comprise sequences as described above that are complementary or share high sequence identity to a nucleic acid sequence of a pathogen, such as a virus, a Coronaviridae or SARS-CoV-2.

A primer designing support software, referred to as ‘LAMP primer designing software, PrimerExplorer’ is available at (http://primerexplorer.jp/e/).

In some cases, the primers are for use in a method for detecting an oligonucleotide of a pathogen in a sample, for example a Coronaviridae such as SARS-CoV-2. In some cases, the set of primers comprises a switch oligonucleotide that comprises or consists of the nucleotide sequence of SEQ ID NO: 8 or a variant having at least 50%, or at least 60%, 70%, 75%, 80%, or 90% sequence identity to SEQ ID NO: 8. In some cases, the complementary primer comprises or consists of the nucleotide sequence of SEQ ID NO: 1 or 2 or a variant having at least 50%, or at least 60%, 70%, 75%, 80%, or 90% or 95% sequence identity to SEQ ID NO: 1 or SEQ ID NO: 2. The complementary primer may be the FIP primer. The primers may further include a reverse inner primer (BIP) comprising the nucleotide sequence of SEQ ID NO: 3 or a variant having at least 50%, or at least 60%, 70%, 75%, 80%, or 90% or 95% sequence identity to SEQ ID NO: 3, a forward outer primer (F3) comprising the nucleotide sequence of SEQ ID NO: 4 or a variant having at least 50%, or at least 60%, 70%, 75%, 80%, or 90% or 95% sequence identity to SEQ ID NO: 4, a reverse outer primer (B3) comprising the nucleotide sequence of SEQ ID NO: 5 or a variant having at least 50%, or at least 60%, 70%, 75%, 80%, or 90% or 95% sequence identity to SEQ ID NO: 5, a forward loop primer (B3) comprising the nucleotide sequence of SEQ ID NO: 6 or a variant having at least 50%, or at least 60%, 70%, 75%, 80%, or 90% or 95% sequence identity to SEQ ID NO: 6, and/or a reverse loop primer comprising the nucleotide sequence of SEQ ID NO: 7 or a variant having at least 50%, or at least 60%, 70%, 75%, 80%, or 90% or 95% sequence identity to SEQ ID NO: 7. In some cases, the set of primers may comprise six further primers having the nucleotide sequences of SEQ ID NOS: 9 to 14, or may use six further primers having the nucleotide sequences of any of the N1 or N17 sets shown in Table 1 of Huang et al. (2020).

### Switch Oligonucleotide

The present invention uses a switch oligonucleotide. The switch oligonucleotide comprises a nucleotide sequence that is complementary to a fragment of one of primers described above. Typically the switch oligonucleotide sequence is complementary to the complementary primer sequence over a region of at least 10, or at least 11, or 12 nucleotides in length, such as from about 10 to about 50, or to 40, or 35, or 30, or 25 nucleotides. In some cases, the switch oligonucleotide may comprise one or more nucleotide analogues, such as analogues described herein, that form double-stranded hybrids with higher stability than natural nucleotides. In this case, the switch oligonucleotide could, in principal, be shorter, such as at least 6, 7, 8 or 9 nucleotides, for example.

Binding of the switch oligonucleotide to the complementary primer is temperature-dependent. Hence, during an amplification reaction (i.e. in solution), the switch oligonucleotide acts as a temperature-dependent switch. The switch oligonucleotide is generally adapted to anneal by hybridization to the complementary primer at temperatures that are below the temperature range for amplification of the oligonucleotide, i.e. below a threshold temperature. This temperature will depend on the DNA polymerase that is used for the reaction. In many cases, the reaction temperature is around 60-65° C. In some cases the switch oligonucleotide substantially anneals to the complementary primer (in solution) at temperatures below about 65° C., or 64° C., or 63° C., or 62° C., or 61° C., or 60° C., or 59° C.

The annealing temperature of an oligonucleotide is determined by a combination of factors including the length of the complementary sequence and the nucleotides (or nucleotide analogues) included or their sequence. The skilled person is able to design an oligonucleotide having a desired annealing temperature using methods and software known in the art.

The switch oligonucleotide may have a sequence that is complementary to the complementary primer across all or part of the F1c, F2, F3, B1c, B2, or B3 regions; or may be complementary to all or part of the F1c region of the FIP, the spacer/loop region, and all or part of the F2 region, i.e. may bridge the region between the F1c and F2 regions and include part or all of both regions; or may be complementary to all or part of the B1c region of the BIP, the spacer/loop region and all or part of the B2 region, i.e. may bridge the region between the B1c and B2 regions and include part or all of them both regions. The switch oligonucleotide does not need to hybridize to the whole of any of the F1c, F2, F3, B1c, B2, or B3 regions, as long as the switch oligonucleotide prevents amplification from the complementary primer when the complementary primer is bound to the switch oligonucleotide. In some cases complementary sequence of the complementary primer is at the 3′end of the primer. For example, in some cases the switch oligonucleotide comprises a sequence that is complementary to at least 10, or at least 11, 12, 13, 14, 15, 16, or 17 3′ terminal nucleotides of the complementary primer. In some cases, the 5′ terminal sequence of the switch oligonucleotide is complementary to the 3′ end terminal sequence of the complementary primer. The switch oligonucleotide may have a sequence that is complementary to the complementary primer across all or part of the F1, F2c, F3c, B1, B2c, or B3c regions.

In some cases, the complementary primer is one of the inner primers, FIP or BIP. In some cases the 5′ terminal sequence of the switch oligonucleotide is complementary to the 3′ end terminal sequence of the FIP of BIP.

In some cases, more than one switch oligonucleotide may be used, wherein (the) additional switch oligonucleotide(s) comprise a sequence, as described above, that is complementary to a different one of the LAMP primers, or a different one of the F1c, F2, F3, B1c, B2, or B3 regions.

The switch oligonucleotide may in some cases have additional nucleotides at the 3′ and/or 5′ end that are not complementary to, and hence do not hybridise to, the complementary primer. For example, the switch oligonucleotide may in some cases have up to 1, or up to 2, 3, 4, 5, 6, 7, 8, 9, 10, 12, 15, 20, 25, 30 or more additional non-complementary nucleotides at the 5′ or 3′ end.

In many cases, the switch oligonucleotide will comprise a sequence that is complementary to the template DNA (either the original template DNA or the looped DNA that is produced during amplification). In such cases it is beneficial to prevent the oligonucleotide from priming additional DNA amplification. Accordingly, in some cases, the switch oligonucleotide is adapted to prevent elongation of or amplification from the switch oligonucleotide, i.e. acting as a primer. Thus, the switch oligonucleotide may comprise a 3′ chain terminator or end blocker oligonucleotide modification (i.e. a terminator moiety). Examples include a 2′, 3′ Dideoxyadenosine (2,3ddA), 2′, 3′ Dideoxycytosine (2,3ddC), 2′, 3′ Dideoxythymidine (2,3ddT) 2′,3′ Dideoxyguanosine (2,3ddG) 3′-Deoxycytidine (3′-dA) 3′-Deoxycytidine (3′-dC) 3′-deoxyGuanosine (3′-dG) and 3′-Deoxycytidine(3′dT). Alternatively, non-nucleoside blockers of 3′-terminus may be used, such as 3′-Spacer C3, 3′-Phosphate, or 1,2-Dihydro-(3H)-pyrrolo[3,2-e]indole-7-carboxylate tripeptide.

In some cases, the 3′ end of the switch oligonucleotide may be modified with a dark quencher molecule. A dark quencher is a molecule that absorbs excitation energy from a fluorophore and dissipates the energy, such that the fluorescent signal is quenched. In some cases, a fluorophore may be included in the reaction mix as an indicator, and the dark quencher helps to reduce baseline fluorescence. An example of a dark quencher that may be used is Iowa Black® (IBRQ).

The switch oligonucleotide is typically used in in a quantity that is in excess of the complementary primer. In some cases the ratio of switch oligonucleotide to complementary primer may be at least or about 1.1:1, 1.2:1, 1.3:1, 1.4:1, 1.5:1, 1.6:1, 1.7:1, 1.8:1, 1.9:1 or 2:1 or more, such as about 1.1:1 to about 10:1, or about 1.2:1 to about 5:1, or about 1.3:1 to about 2:1, or about 1.4:1 to 1.6:1, or about 1.5:1.

The switch oligonucleotide may in general have any suitable feature as for the reaction primers described above.

### DNA Polymerase

Any suitable DNA polymerase may be used in the methods described herein.

The DNA polymerase may be a eukaryotic polymerase. Examples of eukaryotic polymerases that may be used include pol-α, pol-β, pol-δ, pol-ε or any functional variant, analoge, homologe or derivative thereof and any combination thereof.

The DNA polymerase may be a prokaryotic polymerase. Examples of prokaryotic polymerases that may be used include Bacillus stearothermophilus (Bst) DNA polymerase, BcaBEST DNA polymerse (TaKaRa), E. coli DNA polymerase I Klenow fragment, E. coli DNA polymerase I, E. coli DNA polymerase II, E. coli DNA polymerase III, E. coli DNA polymerase IV, E. coli DNA polymerase V, Bacillus stearothermophilus polymerase I large fragment, Bacillus subtilis Pol I large fragment (Bsu polymerase), Listeria monocytogenes DNA polymerase I, Staphylococcus aureus DNA polymerase 1 (Sau) or any functional variant, analoge, homologe or derivative thereof and any combination thereof.

The DNA polymerase may be a bacteriophage polymerase. Examples of bacteriophage polymerases that may be used in the methods described herein include Phi-29 DNA polymerase, T7 DNA polymerase, bacteriophage T4 gp43 DNA polymerase, or any functional variant, analoge, homologe or derivative thereof and any combination thereof.

The DNA polymerase contains strand displacing properties, and typically a high strand displacement activity.

DNA polymerases can use the free 3′-hydroxyl of the invading strand to catalyze DNA synthesis by incorporation of new nucleotides. A number of polymerases can use the 3′-hydroxyl of the invading strand to catalyze synthesis and simultaneously displace the other strand as synthesis occurs. For example E. coli polymerase II or III can be used to extend invaded D-loops. In addition, E. coli polymerase V normally used in SOS-lesion-targeted mutations in E. coli can be used. All of these polymerases can be rendered highly processive through their interactions and co-operation with the p-dimer clamp, as well as single stranded DNA binding protein (SSB) and other components. Other polymerases from prokaryotes, viruses, and eukaryotes can also be used to extend the invading strand.

Many DNA polymerases possess 3′-5′ exonuclease activity, and some also possess 5′-3′ exonuclease activity. 3′-5′ exonuclease activity increases the fidelity of the replication reaction. Accordingly, in some cases a DNA polymerase with 3′-5′ exonuclease activity.

In other cases, using a DNA polymerase with 3′-5′ exonuclease activity and/or 5′-3′ exonuclease activity may be undesirable because it results in digestion of one DNA strand progressively as the polymerase moves forward, rather than displacement. Free oligonucleotides may also be subject to end-dependant degradation when polymerases possessing 3′-5′ exonuclease are employed. Mispriming may also result from oligonucleotides that have been shortened by the 3′-5′ exonuclease activity of polymerases, leading to increased reaction noise. Accordingly in some cases the DNA polymerase does not have 3′-5′ exonuclease activity, and/or does not have 5′-3′ exonuclease activity.

The DNA polymerase may be present at a concentration of between 10,000 units/ml to 10 units/ml, such as between 5000 units/ml to 500 units/ml.

### Reverse Transcriptase

In RT-LAMP, the starting template is RNA, e.g. the RNA genome of a virus. In this case, an reverse transcriptase is used to produce cDNA from the RNA template as an initial step. The cDNA provides the template for amplification.

Any suitable DNA polymerase may be used in the methods described herein.

In many cases, it will be preferable to use a reverse transcriptase that has a similar working temperature as the DNA polymerase that is used. In this way the whole method can be carried out at a single reaction temperature. In some cases the difference between the optimal temperature of the DNA polymerase and reverse transcriptase is up to about +/−10, 5, 4, 3, 3, 2, or 1° C.

The reverse transcriptase may be present at a concentration of between 10,000 units/ml to 10 units/ml, such as between 5000 units/ml to 500 units/ml.

### Reagents

dNTPs

dNTPs, for example dATP, dGTP, dCTP, and dTTP, and derivatives and analogs thereof, find use in the present invention. In leading and lagging strand RPA, ATP, GTP, CTP, and UTP may also be included for synthesis of RNA primers.

In some cases the dNTP may be used at a concentration of between 1 mM to 200 mM of each NTP species.

A mixture of dNTP and ddNTP (ddATP, ddTTP, ddGTP and ddGTP and derivatives and analogs thereof) may be used with ddNTP concentrations at 1/100 to 1/1000 of that of the dNTP (1 μM to 2 mM), for example to generate fragment ladders.

### DNA Destabilizers

Chemicals that destabilize the DNA helix may improve LAMP efficiency. Suitable chemicals may be selected by the skilled person. For example, Notomi et al. (2000) reported that 0.5 to 1.5 M betaine (N,N,N-trimethylglycine) or L-proline, which reduce base stacking, stimulated not only the overall rate of the reaction, but also increased target selectivity with a significant reduction in amplification of irrelevant sequences.

### Buffers

The buffer solution in an (RT-)LAMP reaction may be a Tris-HCl buffer, a Tris-Acetate buffer, or a combination thereof. The buffers may be present at a concentration of between about 10 mM to about 100 mM. A preferred buffer is a Tris-HCl buffer used at a concentration of between about 20 mM to about 30 mM, most preferably 25 mM. The buffered pH may be between 6.5 to 9.0, preferably pH 8.3.

The buffer may contain potassium acetate between about 5 mM to about 50 mM, preferably between about 10 mM to about 40 mM.

Reducing agents may in some cases be included, such as DTT. In some cases the DTT concentration may be between 1 mM and 10 mM, preferably 1 mM.

### Reaction Components

A non-limiting example set of reaction components is described in the examples. Where appropriate different components of the example reactions may be independently selected +/−50%, 40%, 30%, 30%, 10% or 5% for use in methods or compositions of the present invention.

### Drying of Reaction Components

The inventors demonstrate in the Examples herein that a reaction mix comprising the set of primers of the invention and optionally other reaction components can be prepared as a dried reaction mix. The dried primers and reagents offer the advantage of not requiring refrigeration to maintain activity. For example, a tube of primers/reagents may be stored at room temperature. This advantage is especially useful in field conditions where access to refrigeration is limited.

The primers/reagents may be dried by any suitable method. In some cases the primers/reagents may be vacuum-dried. In other cases the primers/reagents may be freeze-dried (lyophilized). Suitable methods for producing vacuum-dried or lyophilized reagents are known in the art.

The primers/reagents may be dried onto the bottom of a tube, or on a bead or any other suitable type of solid support. To perform a reaction, the freeze dried reagents are reconstituted in a buffered solution or water, depending on the composition of the dried reagents. Then a target/template nucleic acid, or a sample suspected to contain a target/template nucleic acid is added. Alternatively, in some cases the reconstitution liquid may also contain the sample nucleic acid. The reconstituted reaction is incubated for a period of time and the amplified nucleic acid, if present, is detected.

The reagents that can be dried before use may include DNA polymerase, reverse transcriptase, dNTPs, ddNTPs, reducing agent, primers, probe(s), stabilizing agent, such as nucleic acid stabilising agent, buffering agents, pH indicator and/or colorimetric indicator, cell lysis reagents, and/or positive or negative control nucleic acid template.

The primers may be any of those described herein.

Stabilizing agents such as dextran, lactose or trehalose sugar may be included in the dried mixture, for example at 20 mM to 200 mM, or 30 mM to 150 mM, or 40 mM to 80 mM in the reconstituted reaction, in order to improve drying performance and shelf life. Bovine serum albumin may be included. If desired, the dried reagents may be stored for up to 2 weeks, 3 weeks, 1 month, 6 months or 1 year or more before use.

The dried mixes may be re-dissolved in water, typically DNase and/or RNase free water, or any other suitable buffer as may be determined by the skilled person in the art. In some cases the pH of the re-dissolved reagents may be adjusted before use.

### Kits

The invention also provides a kit for amplifying or detecting the presence of an oligonucleotide or carrying out a LAMP of RT-LAMP reaction. The kit comprises a set of oligonucleotide primers as described herein.

The kit may further comprise any of the reagents and in any of the concentrations described herein in any suitable combination. In some cases, the kit may comprise DNA polymerase; (ii) reverse transcriptase, if the kit is for use in a method requiring reverse transcription; (iii) a pH indicator and/or colorimetric indicator; (iv) deoxyribonucleotide triphosphates (dNTP); (v) buffer components; and/or (vi) instructions for use.

The reagents of the kit may be dried, as described herein. The reagents may be combined as reagent mix, for example in or on the same solid support, such as a reaction tube. The reagents may be provided in any suitable amount such that when reconstituted the appropriate reagent concentration is achieved.

In some case, the kit is for detecting SARS-CoV-2 or for diagnosing a SARS-CoV-2 infection or Covid-19 in a subject. The kit may comprising a set of six primers and a switch oligonucleotide having the nucleotide sequences of SEQ ID NOs: 2 to 8 or a set of 12 primers and a switch oligonucleotide having the nucleotide sequences of SEQ ID NOs: 2 to 14. The kit may further comprise any of the reagents disclosed herein. In some cases the kit further comprises a DNA polymerase, optionally Bst 2.0 DNA polymerase (New England Biolabs), a reverse transcriptase, optionally a WarmStart reverse transcriptase (New England Biolabs), a colorimetric pH indicator, optionally phenol red, deoxyribonucleotide triphosphates (dNTP), and optionally a buffer, optionally wherein the kit comprises a vacuum-dried reagent mix.

### Reaction Conditions

The amplification reactions may incubated for any suitable length of time. A typical reaction incubation may be at least 5, 10, 15, 20, 25, 30, 35, 40, 45, 50, 55 or 60 minutes, for example between about 5 minutes and 16 hours or more, between about 15 minutes and 1 or 3 hours, between about 20 minutes and 1 hour or 2 hours, or between about 30 minutes and 1 hour.

The incubation may be performed until a desired degree of amplification is achieved. The desired degree of amplification may be 10 fold, 100 fold, 1000 fold, 10,000 fold, 100,000 fold or 1,000,000 fold amplification, or more. LAMP can achieve DNA amplification up to 109 or more copies of the target sequence or 500 ug/ml or more DNA yield (Nagamine et al. (2002)).

The optimal time can be selected by the skilled person using methods known in the art and may in some cases be dependent on the method of detection used, as described below, or the purpose of the amplification. The optimal time will often be that which maximises sensitivity for detecting the presence of a target template RNA or DNA in a test sample, whilst minimising false positives, e.g. due to non-specific or off-target amplification or crss-contamination of samples.

One benefit of LAMP and RT-LAMP is that the reaction may be performed without thermal cycling, such as is needed for PCR. A further advantage of the present invention is that it is less important to maintain a constant temperature or prevent the temperature from dropping below a critical level where non-specific binding or primer dimer formation may occur. Such undesirable binding by at least one reaction primer is prevented or inhibited by, and in competition with, binding to the switch oligonucleotide. Hence, the reaction is preferably, but not essentially conducted at a constant temperature (for example +/−1° C., +/−0.5° C., +/−2° C. or +/−1° C.) at which DNA polymerase and, where appropriate, reverse transcriptase is active. The skilled person can readily select a suitable temperature according to the specific enzymes being used.

In some cases, the reactions may be performed at between 20° C. and 80° C., between 50° C. and 75° C. between 55° C. and 70° C., such as between 60° C. and 65° C.

### Detection of Reaction Products

The products at the end of a LAMP or RT-LAMP reaction are a mixture of DNA concatemers (amplicons) corresponding to the target polynucleotide region. The DNA concatemers are formed from multiple consecutive stem-loops formed by annealing between alternately inverted repeats of the target sequence in the same strand with loops in between.

LAMP amplification or products may be detected by any suitable method as known in the art, including direct or indirect methods of detecting DNA amplification, and hence the presence of the target polynucleotide sequence.

In some cases, detection may be performed by separating the products by electrophoresis, for example on an agarose or PAGE gel. These give rise to a characteristic “ladder” or banding pattern due to the different sized DNA concatemers. Detection may be by staining of the products, for example with ethidium bromide or other intercalating dye, or by Southern blot hybridization with appropriate probes.

In other cases, it may be beneficial to use a method that allows for real-time detection as the reaction proceeds, or for detection immediately after the reaction is completed with minimal additional steps, and/or may requiring minimal specialist equipment or materials Some methods permit analysis using the naked eye. LAMP is very efficient method of DNA amplification and produced large yields of amplified DNA. Hence, in some cases DNA amplification can be determined by observing or measuring white precipitates or the turbidity of white precipitates caused by magnesium pyrophosphate precipitate in solution as a byproduct of amplification in the reaction solution. Alternatively, the turbidity may be measured using photometry/photometric detection approaches.

Pyrophosphate ions strongly bind to metal ions and form insoluble salts. Therefore, an alternative method for detecting the magnesium pyrophosphate is by adding a metal ion, such as manganese, and a metal indicator, e.g. a fluorescent metal detector, such as calcein (Tomita et al. (2008)).

In some cases a colorimetric pH indicator may be used as an indirect indication of the polymerase amplification of the target sequences. Phenol red, for example, will change colour from pink to yellow as a result of DNA amplification in an (RT-)LAMP reaction. The change of colour may be determined by simple observation. Alternatively, the absorbance at one or two different wavelengths may be measured, and optionally the ratio between them calculated determined, wherein the wavelength(s) are chosen to distinguish between the sample colour obtained using control positive and negative samples (Berecher et al. (2020)).

Indirect determination of DNA amplification using a pH indicator may not be suitable in certain cases, for example when comparing samples that might have different pH for reasons other than DNA amplification, or if using buffers that interfere with monitoring the DNA amplification. This effect may be minimised by using DNase and/or RNase free water instead of, or with minimal buffer where possible.

Direct observation measurement of measurement of DNA amplification may be achieved using fluorescence. Intercalating dyes, such as such as SYTO 9, SYBR green, or Qubit BR DNA dye may be used to create a visible color change that can be seen with the naked eye or, where appropriate, under UV light/exposure, or may be measured more accurately using a fluorometer. The fluorescent emission/intensity of a dye that intercalates with or directly labels DNA can be correlated with the number of copies initially present. Hence, this method of detection can also be quantitative.

In other cases a fluorescent probe or label may be used. For example, one or more of the primers that is incorporated into the DNA product (i.e., the FIP, BIP, LF and LB primers) may be conjugated/5′end conjugated with a fluorophore, such as FAM (6-Carboxyfluorescein), as described, for example in Huang et al. (2020). A fluorescent label and a colorimetric indicator as described above may also be combined for increased flexibility and use in different situations, as also described in Huang et al. (2020).

In some cases a dark quencher may be included in the reaction to reduce background fluorescence and increase sensitivity, as further described elsewhere herein.

Another method for visual detection of the LAMP amplicons by the unaided eye uses sequence specific complementary gold-bound ss-DNA (AuNP). Hybridization of the AuNP to the amplified DNA products of (RT-)LAMP salt-induced aggregation of the gold particles and inhibits the normal red to purple-blue color change, thus providing an alternative colorimetric indicator for successful DNA amplification/detection.

In some cases, the method may involve, for example, removing a fraction of the reaction, isolating the unincorporated fraction, and detecting the unincorporated primer. This may be achieved using the large difference in the small size of an unincorporated primer and the much larger size of the amplified product. The isolation of the unincorporated primer may be performed rapidly using size exclusion chromatography such as, for example, a spin column. If a primer is labelled, a monitor procedure comprising a spin column and a measurement (e.g., fluorescence or radioactivity) can be performed in less than one minute. Another alternative for separating elongated primers involves the use of immobilized oligonucleotides. For example oligonucleotides homologous to sequences found uniquely within the amplified DNA sequence can be used to capture nucleic acids produced by primer elongation specifically. These capturing oligonucleotides can be immobilized on a chip, or other substrate. Capture of the elongated oligonucleotides by the capturing oligonucleotides can be performed by RecA protein mediated methods, or by traditional solution hybridizations if necessary.

### Methods of the Invention

In some aspects the present invention relates to a method of loop-mediated isothermal amplification (LAMP) or reverse transcription loop-mediated isothermal amplification (RT-LAMP). The method comprises (a) mixing a set of primers as described herein, including a switch oligonucleotide as described herein, with template DNA or RNA (or a sample suspected of comprising a target template DNA or RNA), deoxyribonucleotide triphosphates (dNTP) and DNA polymerase, and (b) heating the mixture to the working temperature of the DNA polymerase. Provided that the target template DNA or RNA is in fact present, then the method results in amplification of the target DNA, or cDNA corresponding to the target RNA, as described below. Accordingly, the method is for amplifying DNA.

If the initial template nucleic acid is RNA, or if the method is for detecting/determining the presence or absence of an RNA, then a reverse transcriptase is also included. The method then comprises reverse transcription of the template RNA to produce cDNA. The cDNA subsequently provides the initial template for DNA amplification by the DNA polymerase. The method may also include a DNA purification step between the reverse transcription and cDNA amplification steps, but in many cases this step can be omitted (Huang et al. (2020)).

In many cases the DNA polymerase and the reverse transcription will be selected to be active in a similar temperature range. This has the advantage that the whole reaction can be conducted isothermally, essentially at a single temperature. However, in cases where the optimal temperature for activity of the reverse transcriptase is different to the optimal temperature for activity of the DNA polymerase, then the reaction may be incubated at a first temperature for reverse transcription of template RNA to generate cDNA, and then at a second temperature for DNA amplification using the cDNA as initial template. In some cases the duration of incubation at the first temperature may be at least or about 10, 20, 30 seconds or 45 seconds, or at least or about 1, 2, 3, 4, 5, 7 or 10 minutes, for example between 10 or 20 seconds and 1 or 5 minutes. The duration of incubation at the second temperature may be at least 5, 10, 15, 20, 25, 30, 35, 40, 45, 50, 55 or 60 minutes, for example between about 5 minutes and 16 hours or more, between about 15 minutes and 1 or 3 hours, between about 20 minutes and 1 hour or 2 hours, or between about 30 minutes and 1 hour. The first and second temperatures will be determined by the working temperatures for the reverse transcriptase and DNA polymerase, respectively. Before the reaction is started, all samples are preferably kept on ice.

In some cases, the method of the invention is for detecting the presence of a specific target DNA or RNA in a sample, or for determining whether or not a specific target DNA or RNA is present in a sample. The specific target DNA is the template DNA that is amplified, if present, using the method. In the case of an initial RNA template, cDNA is generated using the target RNA as initial template and the cDNA sequence corresponding to the target RNA is the DNA that is amplified during the reaction. The target DNA or RNA corresponds to, or is defined by, the region between and including the F2c and B2c regions that hybridize to the FIP and BIP primers respectively, as described herein. The method may be capable of detecting less than 100, or less than 80 or less than 50, or less than 40, less than 30, or less than 20 copies of the target viral RNA.

If the DNA or RNA is present in the sample then the target DNA, or the cDNA corresponding to the target RNA, is amplified. Amplification of the target DNA or cDNA may be detected to confirm the presence of the target DNA or RNA in a sample used to provide the initial template DNA or RNA for the reaction. Conversely, if the reaction does not result in the amplification of DNA, this can indicate the absence of the target DNA or RNA in a sample used as initial template for the reaction. In some cases, false positives can result from sample (cross-) contamination of non-specific or off-target amplification, although this is reduced using the present invention as described herein. The occurrence of false positives can also be reduced by other methods, such as calibrating the length of the incubation/reaction and minimising sample handling. In some cases, the presence or absence of the target DNA or RNA in the sample may be determined by the detection of a pre-determined threshold quantity, or indication thereof as discussed herein, of amplification. For example, a positive sample could be defined as having values at least 2×, or at least 3×, 4×, 5×, 7× or 10× the standard deviation of a negative control, and/or a negative control could be deined as having less than 2×, or less than 3×, 4×, 5×, 7× or 10× the quantity as a negative control. Detection of DNA amplification below the threshold may be the result of contamination or off-target/non-specific amplification, and so may in suitable cases be determined as a negative result or inconclusive.

In some cases any of the methods of the invention described herein may further comprise detecting the presence of the target DNA or RNA in the sample. Any suitable detection method described herein, or otherwise known in the art may be used. In other cases the method may comprise detecting step, but if amplification is not detected, or I detected below a pre-determined threshold as described above, then a negative result, i.e. the absence of the target DNA or RNA in the sample intended to provide the template, may be determined.

In some cases the method may be for detecting/determining the presence/absence of a DNA or RNA associated with a particular disease, condition or infection. For example, in some cases the method may be used for detecting a genetic mutation in a sample, or in a subject, or a suitable biological sample obtained from a subject, wherein the mutation is associated with a disease or condition, such as a cancer or other genetic condition. In other cases, the target DNA or RNA may be associated with a pathogen and the method is for detecting/determining the presence/absence of the pathogen in a sample, or in a subject, or a suitable biological sample obtained from a subject or for detecting or diagnosing an infection with the pathogen, or a previous infection by the pathogen in a subject. Typically the target DNA or RNA is comprised in the genome of the pathogen. If the target DNA/RNA is detected or determined to be present (optionally above a pre-determined threshold as described above), then the method may in some cases further comprise diagnosing a relevant disease, condition or infection in the subject. In some cases, the method may further comprise selecting and/or administrating a suitable treatment to the subject.

In other cases, the method may comprise using a suitable sample obtained from a subject as the template RNA or DNA, wherein the sample is suspected of comprising the target DNA or RNA, wherein the target RNA or DNA is associated with a disease, condition, infection or pathogen, wherein the method comprises detecting/determining that the method does not amplify the target DNA or cDNA (or amplified DNA to a quantity below a pre-set threshold), and further determining that the subject does not have the disease, condition or infection with the pathogen.

In some cases, the method of the invention is a method of detecting the presence of DNA or RNA associated with a disease, condition, pathogen or infection in a biological sample obtained from a subject, or of diagnosing a disease, condition or infection in a subject, based on the detection of a target DNA or RNA (or a pre-determined threshold quantity of a target DNA or RNA, or amplified DNA) in a biological sample obtained from the subject, wherein the target DNA or RNA is associated with the disease, condition, a pathogen or an infection.

The individual may be a human or a non-human animal. Non-human animals include, but are not limited to, rodents (including mice and rats), and other common laboratory, domestic and agricultural animals, including rabbits, dogs, cats, horses, cows, sheep, goats, pigs, chickens, amphibians, reptiles etc.

Any suitable biological sample may be used. Specifically, the sample should be one in which the target DNA or RNA is present in a subject that has the relevant disease, condition or infection. Examples include samples obtained from a nasal, pharyngeal, oro-nasopharyngeal nasal or throat swab.

In some cases, the sample may be processed prior to being used in the LAMP reaction. For example, in some cases the sample may be processed to extract DNA or RNA, and the extracted DNA or RNA may be used as the template for the LAMP reaction. In other cases, extraction of DNA or RNA prior to use as template in the LAMP reaction may not be necessary, as demonstrated in Example 6 below. In other cases, the method may be a one-step detection method, using any suitable biological or clinical sample as described herein as template, and comprising cell lysis, RNA extraction, RNA reverse transcription, and LAMP amplification in a single one step reaction. Such a method is described in Huang et al. 2020. The method may be conducted at least 55° C., at least 60° C., or at least 65° C. The method may take 5 to 20, for example 5 to 10 minutes longer than RT-LAMP conducted using extracted RNA because cells take about this tie to lyse and release nucleic acids.

The pathogen may in some cases be a virus, a bacteria, a fungus, or a protozoa. In some cases the pathogen may be one having a viral genome. The virus may be a Coronaviridae, such as SARS-CoV-2. The target RNA may be any suitable fragment of a Coronaviridae or SARS-CoV-2 genome.

In some cases, the method is a method of detecting SARS-CoV-2 in a sample, or diagnosing a SARS-CoV-2 infection or COVID-19 in a subject, the method comprising (i) obtaining the sample, or obtaining a biological sample from the subject; (ii) reverse transcription to produce cDNA from RNA in the sample; (iii) amplification of the reverse transcribed cDNA using a set of six oligonucleotide primers having the nucleotide sequences of SEQ ID NOs: 2 to 7, and a switch oligonucleotide having the nucleotide sequence of SEQ ID NO: 8; (iv) detecting the amplified DNA; and (v) determining the presence of SARS-CoV-2 or diagnosing a SARS-CoV-2 infection or COVID-19 in the subject.

In some cases, the cDNA amplification step may further use six further primers having the nucleotide sequences of SEQ ID NOs: 9 to 14, or may use six further primers having the nucleotide sequences of any of the N1 or N17 sets shown in Table 1 of Huang et al. (2020).

In other cases, the method is a method of determining the absence of SARS-CoV-2 in a sample, or a negative diagnosis of a SARS-CoV-2 infection or COVID-19 in a subject, the method comprising (i) obtaining the sample, or obtaining a biological sample from the subject; (ii) reverse transcription; (iii) a LAMP reaction using a set of six oligonucleotide primers having the nucleotide sequences of SEQ ID NOs: 2 and 4 to 7, and optionally six further primers primers having the nucleotide sequences of SEQ ID NOs: 9 to 14; and a switch oligonucleotide having the nucleotide sequence of SEQ ID NO: 1; (iv) determining that no DNA or an amount of DNA below a pre-defined threshold amount is amplified; and (v) determining the absence of SARS-CoV-2 in a sample, or a negative diagnosis of a SARS-CoV-2 infection or COVID-19 in a subject.

In some cases, the method is a method of reducing false positives in the detection of an target RNA or DNA or a pathogen in a sample, or the diagnosis of a disease, condition or infection, such as any described herein. In particular, the method reduces the incidence or likelihood of obtaining a false positive, or increases the statistical certainty that a positive result is a true positive, when compared to an otherwise identical method that does not include use of a switch oligonucleotide as described herein.

### EXAMPLES

### Example 1—Temperature Dependent Oligo Switch Stabilises RT-LAMP Assay

To address the problem of self- and off-target amplification, we introduced a short oligonucleotide designated as a switch, whose sequence is complementary to one of the primers used in the LAMP reaction and 3′-end was modified by a terminator analog (e.g. 3′-dA chain terminator) or a dark quencher molecule—Iowa Black® RQ (IBRQ). The switch serves as a temperature-dependent switch to bind an essential primer of LAMP (e.g. FIP), preventing from non-specific amplification and primer dimers. When the temperature is lower than working temperature of LAMP (e.g. 65° C.), the switch binds a primer to prevent unspecific dimer amplification. At the working temperature, the switch dissociates the primer, allowing it to bind target RNA/DNA for reverse transcription and amplification of nucleic acids.

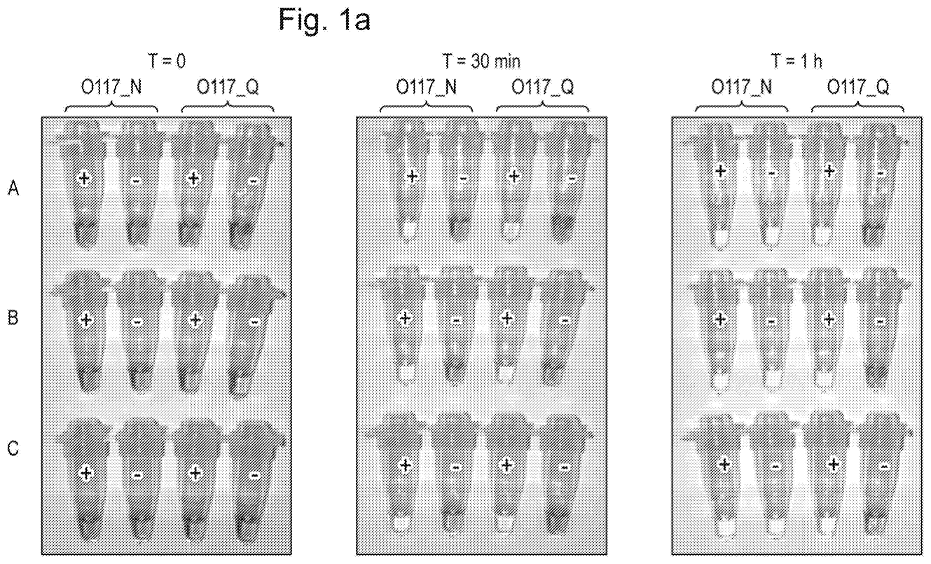

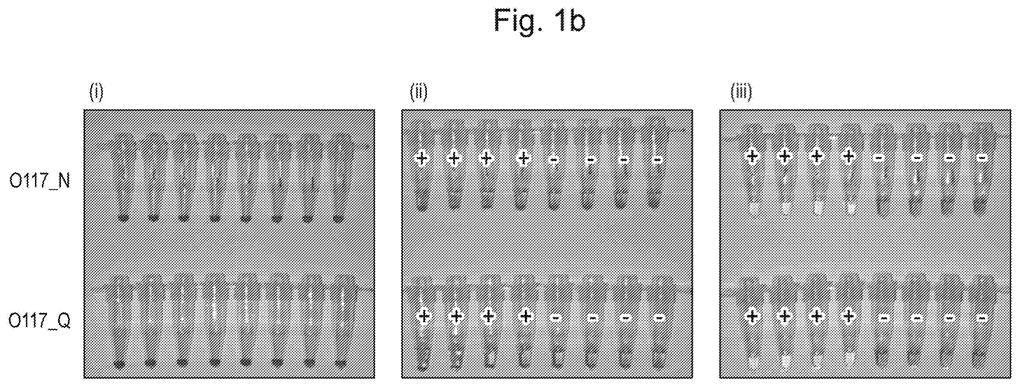

O117 primer set (Huang et al. 2020) was used to evaluate the performance of the switch. Two preparations of the primer mix were used throughout this study: O117_N contains the original six LAMP primers only, and O117_Q contains the six LAMP primers and a switch oligonucleotide (Table 1). The blank controls without target template have been previously shown to result in false positive due to non-template amplification. In this study, a 12-bp switch with 3′-end modification with IBRQ (IDT, UK) was designed to bind FIP and block self or off-target amplification when the temperature is lower than LAMP working temperature. O117_N or O117_Q primers were used to test Synthetic SARS-CoV-2 RNA Control 2—MN908947.3 (Twist Bioscience) and control human cDNA (Sigma-Aldrich UK) through a prolonged incubation of up to 1 hour. WarmStart Colorimetric Master Mix (New England Biolabs, UK) was used for the LAMP assay. After incubating at 65° C. for 30 minutes, both O117_N and O117_Q LAMP primers, showed positive (yellow) to 200 copies of Synthetic RNA Control 2, and negative (pink) to human cDNA control (FIG. 1a). However, the O117_N primer mix turned yellow within one hour of incubation with control human cDNA, while the O117_Q mix maintained a pink colour throughout the experiment (FIG. 1a).

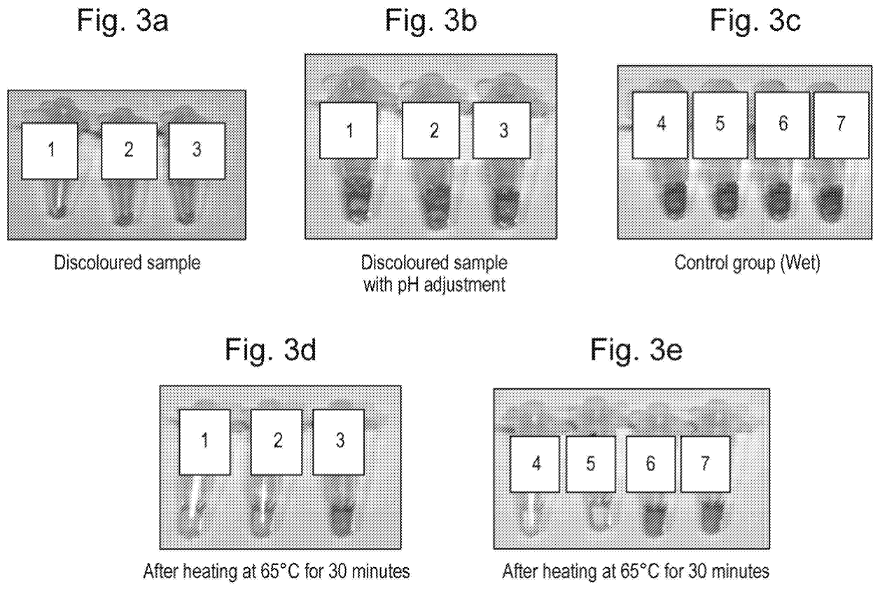

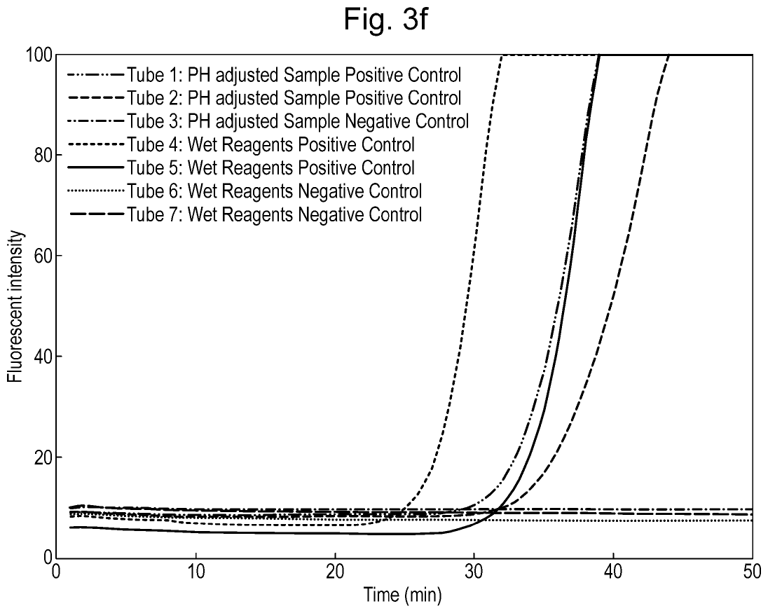

We also showed that O117_N primer set in an unstable heating could also generate false positive result, whilst O117_Q can perform reliably (See FIG. 3). The results suggest that a switch in O117 primers could stabilise the LAMP assay for SARS-CoV-2 and generate reliable results without false positive.

TABLE 1
Primer sequences used in O117_N and O117_Q primer
mix.

| | | SEQ |
|---|---|---|
| O117 | | ID |
| Primer | Sequence (5′-3′) | NO |
| FIP | GGTTTTCAAGCCAGATTCATTATGGATGTCACAATTCAG | 2 |
| | AAGTAGGA | |
| BIP | TCTTCGTAAGGGTGGTCGCAGCACACTTGTTATGGCAAC | 3 |
| F3 | CCCCAAAATGCTGTTGTT | 4 |
| B3 | TAGCACGTGGAACCCAAT | 5 |
| LF | TCGGCAAGACTATGCTCAGG | 6 |
| LB | TTGCCTTTGGAGGCTGTGT | 7 |
| Switch | GGCTTGAAAACC-IBRQ | 8 |

*IBRQ: Iowa Black Dark Quenchers

### Example 2—Vacuum-Drying Improves Reaction Mix Storage and Transport

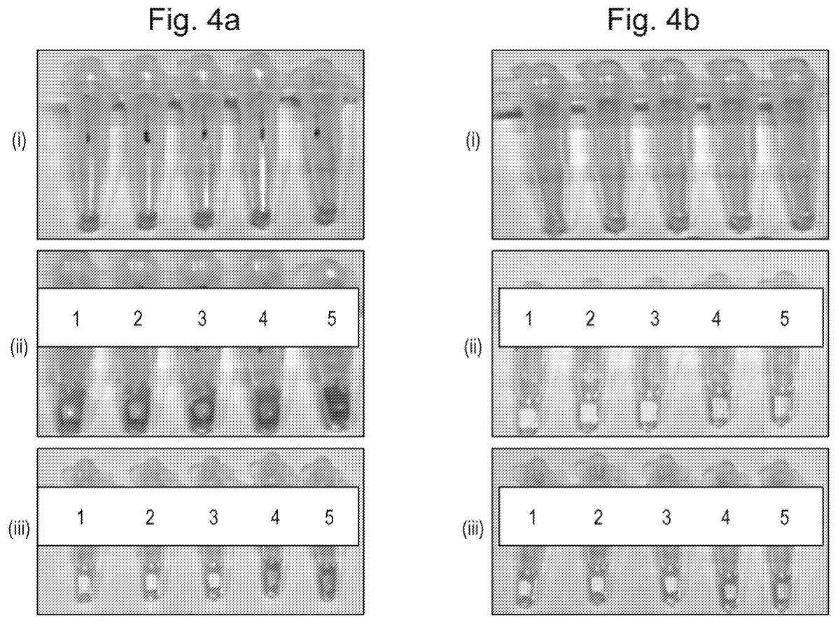

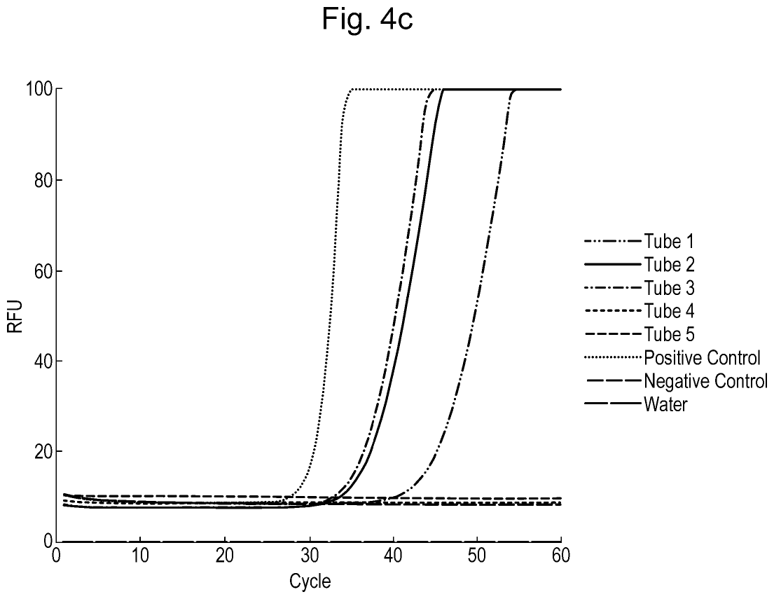

One limitation of the deployability of RT-LAMP to developing countries and rural area is the need for cold-chain transport of reagents. To overcome this limitation, we vacuum-dried a complete reagent mix, including Bst2.0 DNA polymerase, WarmStart reverse transcriptase, colorimetric indicator, dNTPs, primers, and buffer, in one PCR tube to make a dry and ready-use reaction mixture. The stability of the dried reaction mix was assessed by storing at room temperature for 14 days before RT-LAMP assay was performed (FIG. 1b and FIGS. 3 and 4). On Day 3, the dried mixes were first re-dissolved with 20 μL DNase/RNase free water followed by the addition of positive control (Synthetic SARS-CoV-2 RNA Control 2) and negative control (1 μg human genome cDNA) (FIG. 1b ii). After the RT-LAMP reaction, a clear colour difference was shown between the positive controls and negative controls, indicating a high retention of enzymatic activity after 3 days of dried storage at room temperature (FIG. 1b iii). Dried reaction mixes stored at room temperature for 14 days showed similar enzymatic activity, despite a colour change since day 7 in the dry kits (See FIGS. 3 and 4).

### Example 3—Optimised RT-LAMP Reaction Mix Maintains High Sensitivity and Specificity

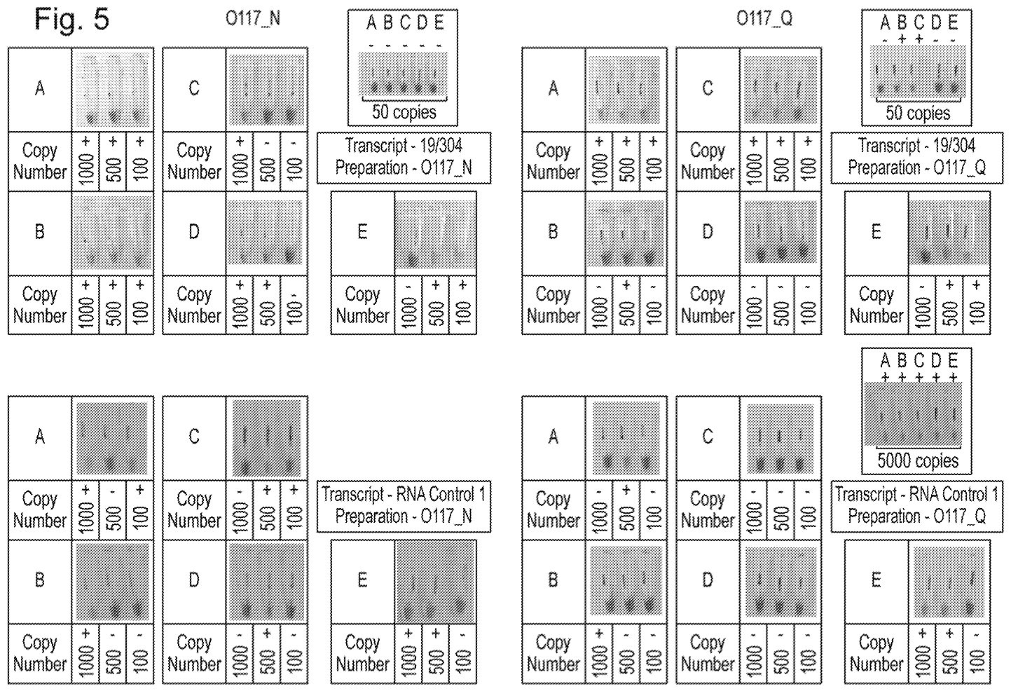

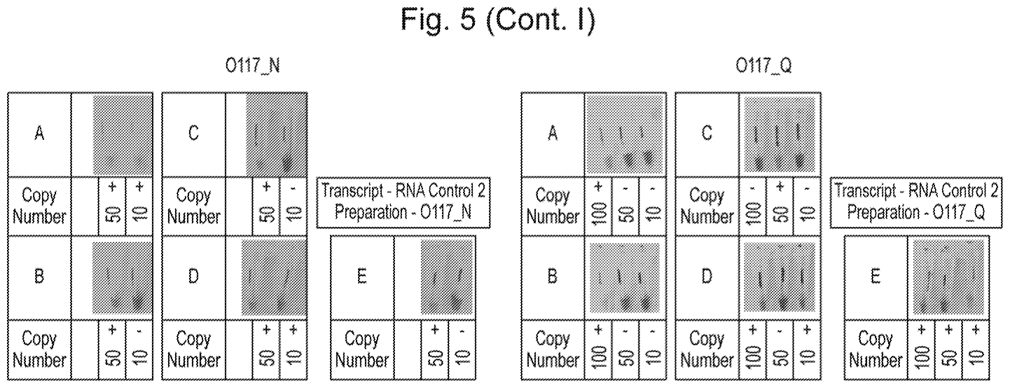

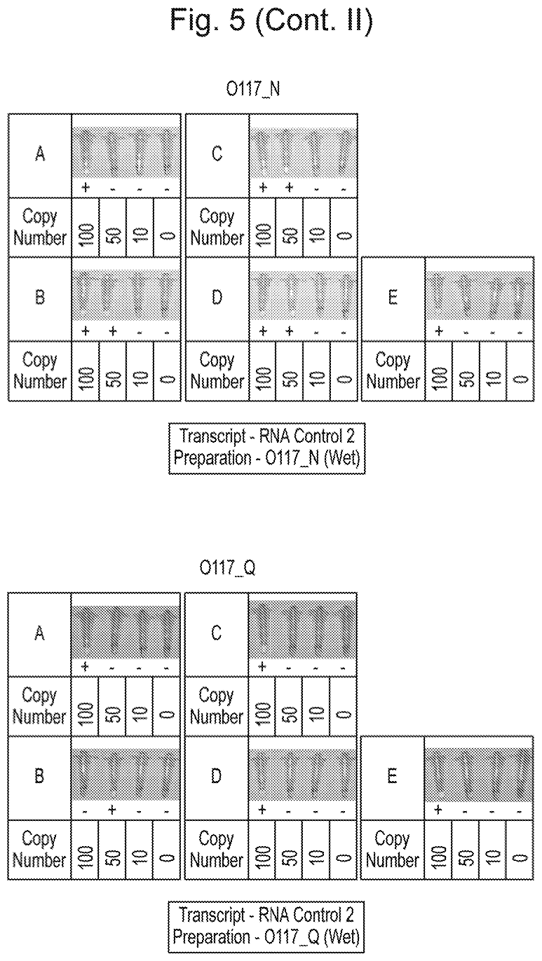

The 50% endpoints of detection (50EP) of dried kits of the RT-LAMP assay containing O117_N and O117_Q were determined using three full-length transcripts serially diluted in buffer AVE (Table 2). The RNA templates were research reagent 19/304 (NIBSC), Synthetic SARS-CoV-2 RNA control 1—MT007544.1 and Synthetic SARS-CoV-2 RNA control 2 MN908947.3 (Twist BioScience). The testing results are summarised in Table 2 and FIG. 5. It shows that 50EP against 19/304 were 71 (O117_Q) and 89 (O117_N) copies per reaction (25-μl). The EP50 to Synthetic RNA Control 1 were 131 and 224 copies per reaction for O117_Q and O117_N, respectively. Synthetic RNA Control 2 demonstrated 50EP of 60 and 13 copies per reaction for O117_Q and O117_N, respectively. Additionally, we also tested the 50EP for detection of Synthetic RNA control 2 in wet reaction mixes. The level of detectable RNA for O117_Q and O117_N in wet reaction mix were 71 and 42 copies. The 50EP for dried reaction mixes containing O117_Q targeting 19/304 and Synthetic RNA Control 2 had a mean of 65 copies (95% CI: 57.3-72.9). The 50EP for dried reaction mixes containing O117_N and targeting 19/304 and Synthetic RNA control 2 had a mean of 51 copies (95% CI: −1.6-104.0). These results suggest that the oligo switch in O117 primers has a minimal impact on the LAMP sensitivity for viral RNA detection and 50EP is 60-131 per 25-μl reaction.

TABLE 2
The 50% end-point of detection using
full-length transcript standards.

| | Dried Kit with | Dried Kit with |
|---|---|---|
| Full-length transcript | O117_Q* | O117_N* |
| 19/304 | 71 | 89 |
| RNA Control 1 | 131 | 224 |
| RNA Control 2 | 60 | 13 |
| RNA Control 2 | 71 | 42 |
| (Wet kit) | | |

*the total copy number per 25-μl reaction

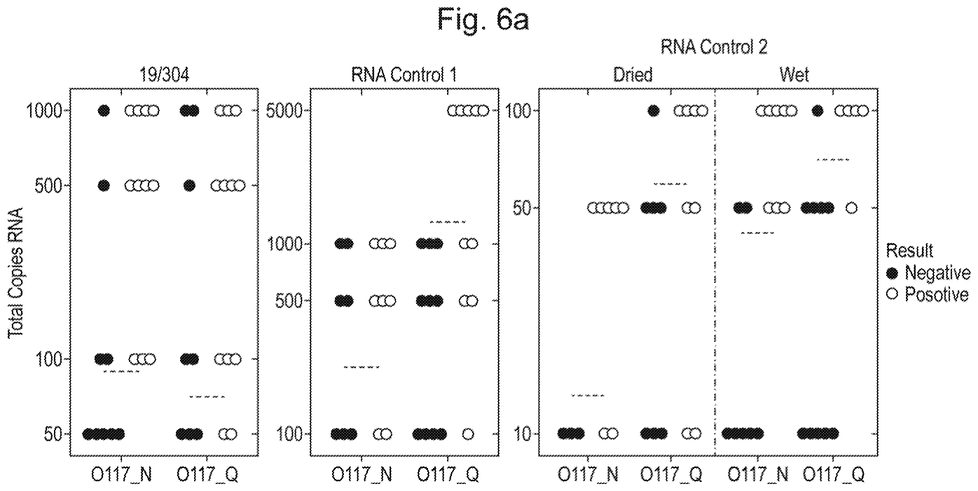

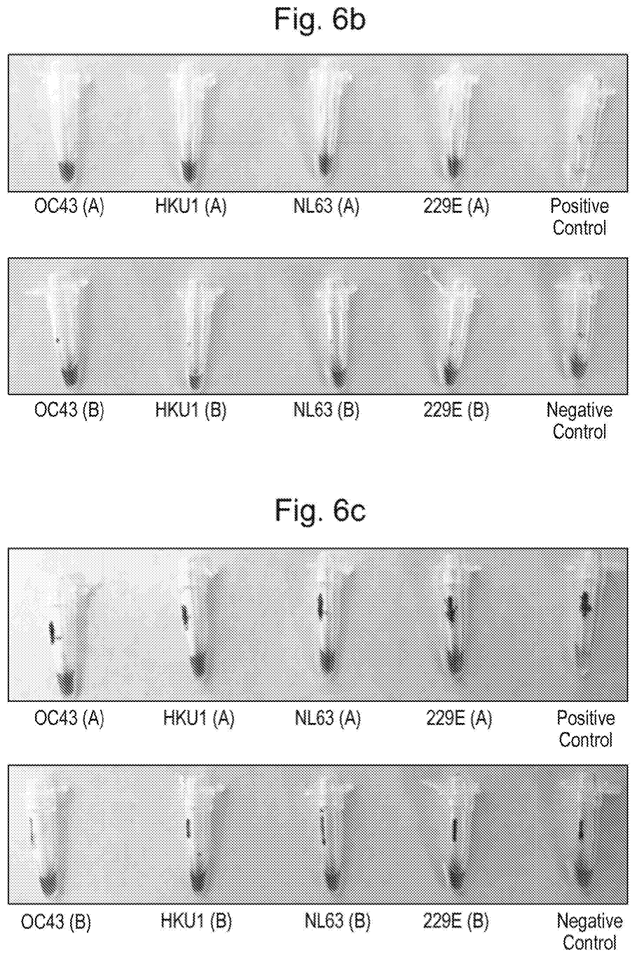

The ability of the optimised RT-LAMP assay to discriminate SARS-CoV-2 from seasonal coronaviruses was assessed using RNA extracted from four worldly-common human coronavirus OC43, HKU1, 229E and NL63 (https://www.cdc.gov/coronavirus/types.html). The RNA samples from infected patient samples positive for beta-coronaviruses (OC43 and HKU1) and alpha-coronaviruses (229E and NL63) were used for specificity testing of O117_Q LAMP assay. Neither replicate of any of the seasonal coronaviruses tested by O117_Q produced a positive result, even using an extended reaction time (40 instead of 30 minutes) (FIGS. 6b and 6c). It suggests that O117_Q primer set had a high specificity to test SARS-CoV-2, without false positive result to against related coronaviruses for other respiratory illness

### Example 4—Clinical Validation with RNA Extract from Patient Samples

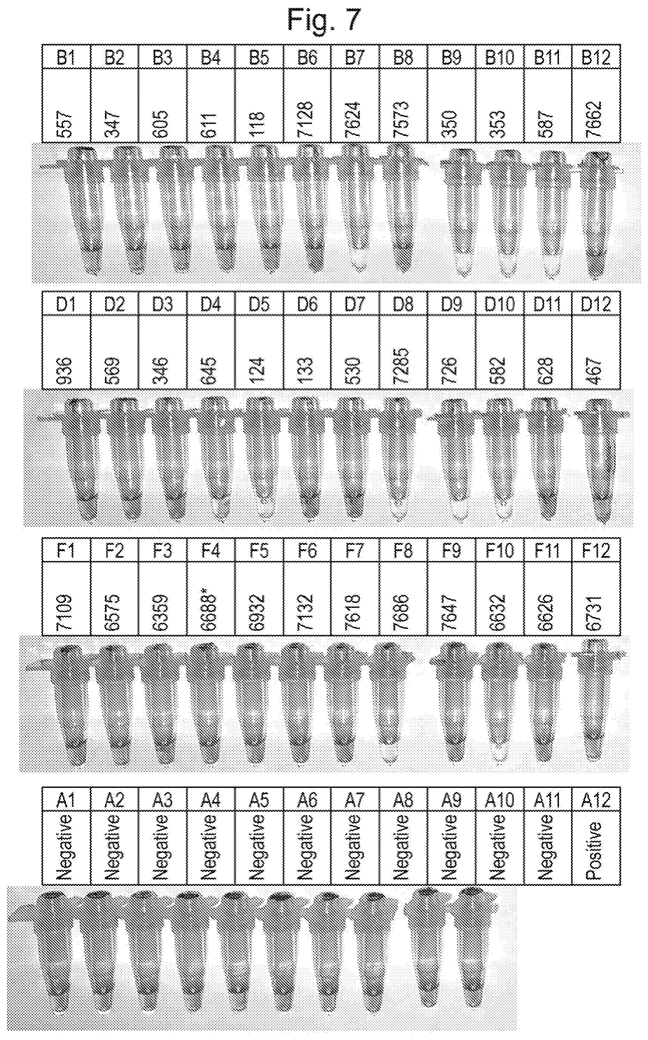

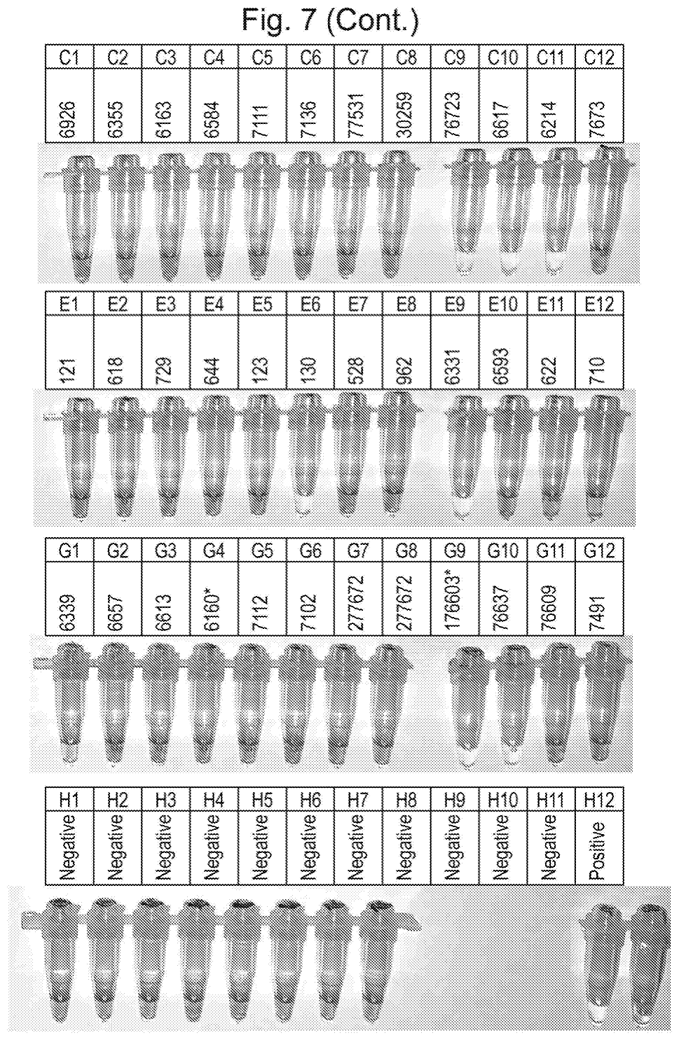

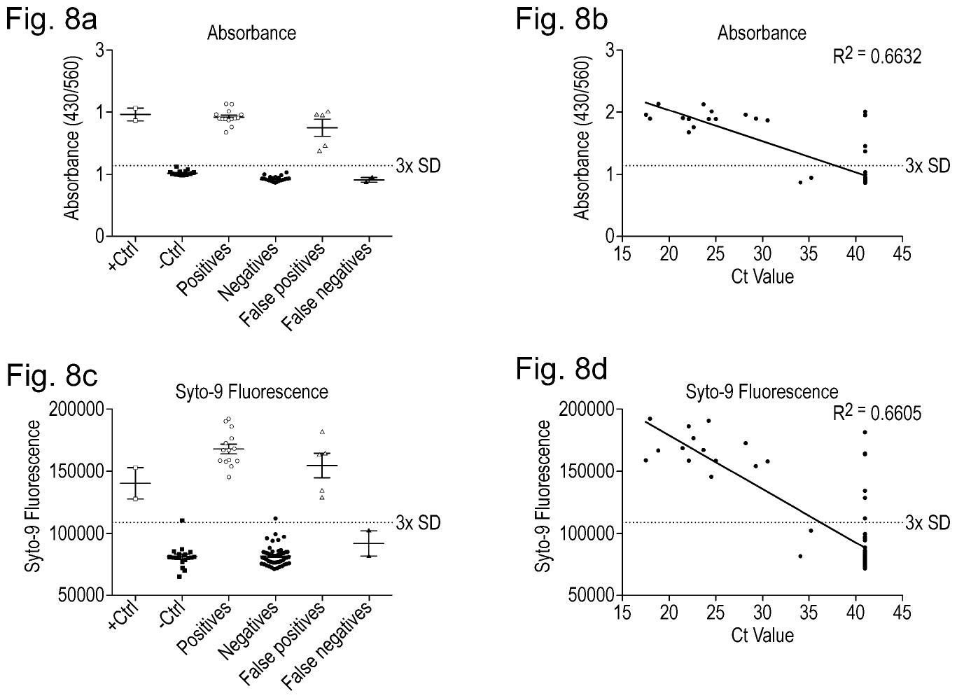

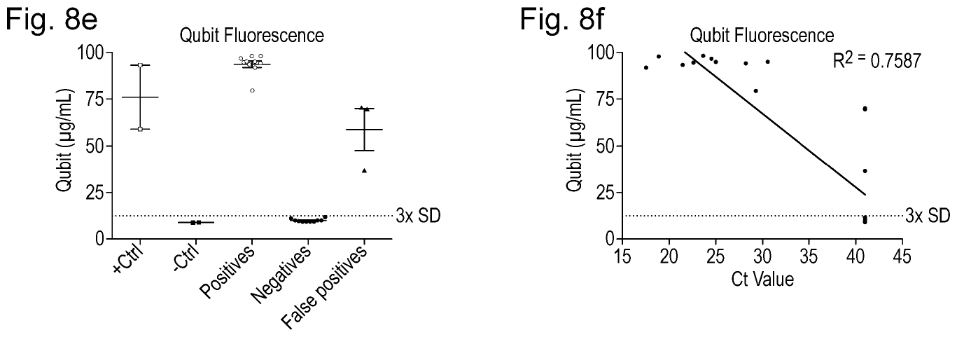

The performance of wet reaction mix containing O117_Q was evaluated using SARS-CoV-2 RNA extracts from nasopharyngeal swab samples. The clinical swab samples from 72 patients were assessed by both RT-qPCR and RT-LAMP in parallel and the results are shown in Table 3. Eighteen negative controls and two positive controls show the expected results (FIG. 7). Visually-assessed colorimetric analysis revealed that the RT-LAMP assay was consistent to the fluorescent reading (FIG. 8). The agreement between RT-LAMP and RT-qPCR assay was 14/16 SARS-CoV-2 positive samples (positive percentage agreement, PPA=87.5%) and 51/56 SARS-CoV-2 negative samples (negative percentage agreement, NPA=91.10%), demonstrating an overall percentage agreement (OPA) of 90.3% (65/72 matching samples) (FIG. 7). The two false negative samples had high Ct values (Ct value >34), which is beyond the estimated detection limit of our RT-LAMP assay for the viral RNA detection.

TABLE 3
Performance of the improved RT-LAMP assay in detecting
SARS-COV-2 from RNA extract isolated from patient samples.
RT-LAMP 22.05.2020

| | | | RT-LAMP |
|---|---|---|---|
| Sample | RT-qPCR Result | Ct Value | Result |
| B1 | NOT DETECTED | — | NEGATIVE |
| B2 | NOT DETECTED | — | NEGATIVE |
| B3 | NOT DETECTED | — | NEGATIVE |
| B4 | NOT DETECTED | — | NEGATIVE |
| B5 | NOT DETECTED | — | NEGATIVE |
| B6 | NOT DETECTED | — | NEGATIVE |
| B7 | DETECTED | 24.54 | POSITIVE |
| B8 | NOT DETECTED | — | NEGATIVE |
| B9 | DETECTED | 21.45 | POSITIVE |
| B10 | DETECTED | 29.29 | POSITIVE |
| B11 | DETECTED | 25.02 | POSITIVE |
| B12 | NOT DETECTED | — | NEGATIVE |
| C1 | NOT DETECTED | — | NEGATIVE |
| C2 | NOT DETECTED | — | NEGATIVE |
| C3 | NOT DETECTED | — | NEGATIVE |
| C4 | NOT DETECTED | — | NEGATIVE |
| C5 | NOT DETECTED | — | NEGATIVE |
| C6 | NOT DETECTED | — | NEGATIVE |
| C7 | NOT DETECTED | — | NEGATIVE |
| C8 | NOT DETECTED | — | NEGATIVE |
| C9 | DETECTED | 23.69 | POSITIVE |
| C10 | DETECTED | 17.53 | POSITIVE |
| C11 | DETECTED | 30.54 | POSITIVE |
| C12 | NOT DETECTED | — | NEGATIVE |
| D1 | NOT DETECTED | — | NEGATIVE |
| D2 | NOT DETECTED | — | NEGATIVE |
| D3 | NOT DETECTED | — | NEGATIVE |
| D4 | NOT DETECTED | — | POSITIVE |
| | | | (False positive) |
| D5 | NOT DETECTED | — | POSITIVE |
| | | | (False positive) |
| D6 | NOT DETECTED | — | NEGATIVE |
| D7 | NOT DETECTED | — | NEGATIVE |
| D8 | NOT DETECTED | — | POSITIVE |
| | | | (False positive) |
| D9 | DETECTED | 18.86 | POSITIVE |
| D10 | DETECTED | 22.61 | POSITIVE |
| D11 | NOT DETECTED | — | NEGATIVE |
| D12 | NOT DETECTED | — | NEGATIVE |
| E1 | NOT DETECTED | — | NEGATIVE |
| E2 | NOT DETECTED | — | NEGATIVE |
| E3 | NOT DETECTED | — | NEGATIVE |
| E4 | NOT DETECTED | — | NEGATIVE |
| E5 | NOT DETECTED | — | NEGATIVE |
| E6 | NOT DETECTED | — | POSITIVE |
| | | | (False positive) |
| E7 | NOT DETECTED | — | NEGATIVE |
| E8 | NOT DETECTED | — | NEGATIVE |
| E9 | DETECTED | 28.2 | POSITIVE |
| E10 | DETECTED | 34.07 | NEGATIVE |
| | | | (False negative) |
| E11 | NOT DETECTED | — | NEGATIVE |
| E12 | NOT DETECTED | — | NEGATIVE |
| F1 | NOT DETECTED | — | NEGATIVE |
| F2 | NOT DETECTED | — | NEGATIVE |
| F3 | NOT DETECTED | — | NEGATIVE |
| F4 | NOT DETECTED | — | NEGATIVE |
| F5 | NOT DETECTED | — | NEGATIVE |
| F6 | NOT DETECTED | — | NEGATIVE |
| F7 | NOT DETECTED | — | NEGATIVE |
| F8 | DETECTED | 22.11 | POSITIVE |
| F9 | DETECTED | 35.24 | NEGATIVE |
| | | | (False negative) |
| F10 | DETECTED | 17.96 | POSITIVE |
| F11 | NOT DETECTED | — | NEGATIVE |
| F12 | NOT DETECTED | — | NEGATIVE |
| G1 | NOT DETECTED | — | POSITIVE |
| | | | (False positive) |
| G2 | NOT DETECTED | — | NEGATIVE |
| G3 | NOT DETECTED | — | NEGATIVE |
| G4 | NOT DETECTED | — | NEGATIVE |
| G5 | NOT DETECTED | — | NEGATIVE |
| G6 | NOT DETECTED | — | NEGATIVE |
| G7 | NOT DETECTED | — | NEGATIVE |
| G8 | NOT DETECTED | — | NEGATIVE |
| G9 | DETECTED | 24.24 | POSITIVE |
| G10 | DETECTED | 22.11 | POSITIVE |
| G11 | NOT DETECTED | — | NEGATIVE |
| G12 | NOT DETECTED | — | NEGATIVE |

### Example 5—Quantitative Assessment of Colorimetric Readout

The colorimetric RT-LAMP assay might be subjective. To overcome the ambiguity and potential for inter-user and inter-lab variation when using a visually-assessed colorimetric readout, we sought to establish quantitative measurements for differentiating positive and negative samples, as seen in FIG. 8. The first method uses the ratio of the absorbance at 430 nm and 560 nm to quantitatively assess the color change of the same 72 clinical samples (FIG. 5). The result is consistent to the colorimetric readout by the naked eyes (FIGS. 8a and 8b). SYTO9 and Qubit fluorescent-based approaches were also used to detect the products of the LAMP reaction. Specifically, LAMP products were stained with SYTO9 and using a microplate reader (FIGS. 8c and 8d), or stained with Qubit BR DNA dye and read with a Qubit fluorometer (FIGS. 8e and 8f). Positive samples are defined as having values 3× the standard deviation of negative controls (n=18). As the Qubit is a portable, compact, and accurate readout of fluorescence, we randomly selected a set of 10 positive and negative samples as identified previously using RT-qPCR to verify its applicability for POCT. We established a cutoff value of 12.5 μg/mL Qubit reading based on the distribution of negative controls and negative samples. Overall, all three methods demonstrated good agreement with the pH-dependent colorimetric RT-LAMP readout, validating their use as a quantitative measurement to indicate RT-LAMP outcomes. Specifically, absorbance, Syto-9, and Qubit revealed a 2.1, 2.1 and 9.4 fold change between SARS-CoV-2 positive and negative patient samples, respectively. Qubit reagents and fluorometer reading demonstrated a good performance.

### Example 6—Detection of SARS-CoV-2 Directly from Oro-Nasopharyngeal Swab Specimens

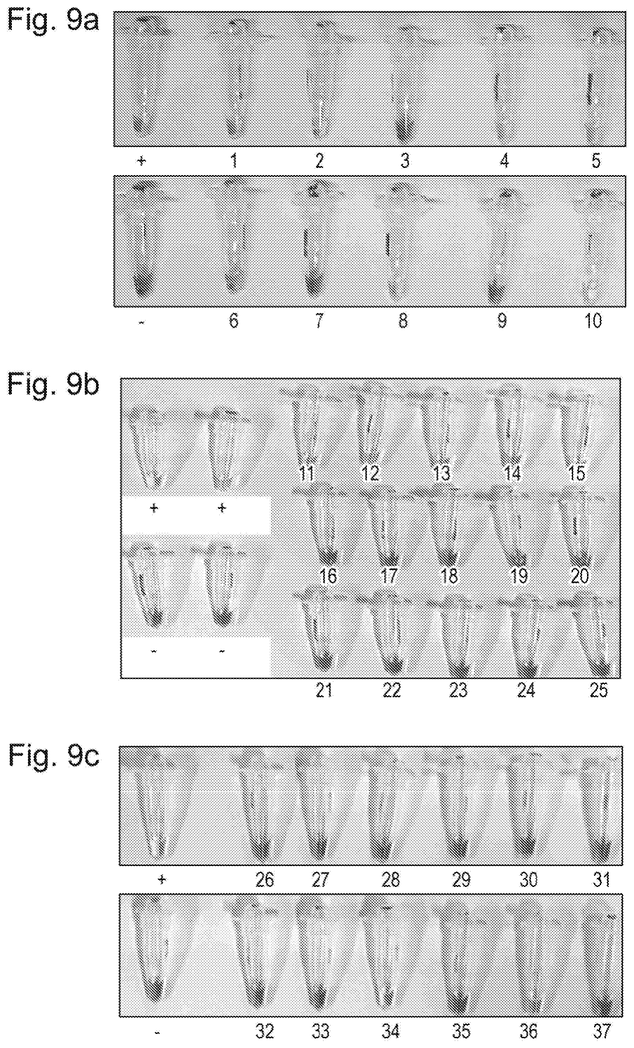

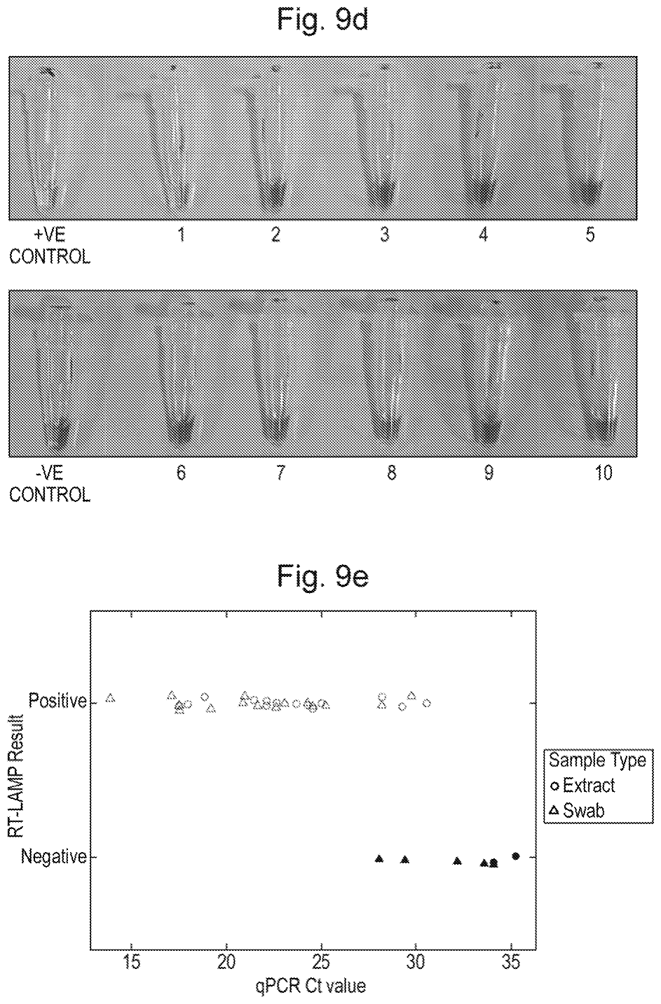

The RT-LAMP dried O117_Q kits were used to directly test oro-nasopharyngeal swabs without any pre-treatment or RNA extraction. Oro-nasopharyngeal swabs from 47 patients were either deposited in Universal Transfer Medium (COPAN Diagnostic, USA) or in 0.85% saline solution for direct RT-LAMP and RT-qPCR in parallel (See Table 4). Direct RT-LAMP was used to test swab samples in 45 min. The same samples were processed with RNA extraction, and then the RNA was used to RT-qPCR. Overall, compared to RT-qPCR results, the dried O117_Q kits displayed a positive percentage agreement (PPA) of 75% (15/20 SARS-CoV-2 positive samples) and a negative percentage agreement (NPA) of 100% (27/27 SARS-CoV-2 negative samples), with an OPA of 89.4% (42/47 matching samples) (FIG. 9a-d). The false negative samples had Ct values of 28.07, 32.2, 33.6, 34.1 and 29.4. The performance of the optimised RT-LAMP assay on both clinical RNA extract and swab samples are summarised in FIG. 9e and Table 5.

TABLE 4
Performance of RT-LAMP on oro-nasopharyngeal swab samples
without any pre-treatment and RNA extraction.

| | | | | |
|---|---|---|---|---|
| Tube | | Previous RT-PCR | | |
| Number | Sample Number | Result | CT Value * | RT-LAMP Result |

RT-LAMP 1 May 2020

| | | | | |
|---|---|---|---|---|
| 1 | SWAB.1.5.1 | DETECTED | 17.13 (20.43) | POSITIVE |
| 2 | SWAB.1.5.2 | DETECTED | 28.2 (31.5) | POSITIVE |
| 3 | SWAB.1.5.3 | DETECTED | 33.57 (36.87) | NEGATIVE |
| | | | | (False negative) |
| 4 | SWAB.1.5.4 | DETECTED | 23.06 (26.36) | POSITIVE |
| 5 | SWAB.1.5.5 | DETECTED | 24.54 (27.84) | POSITIVE |
| 6 | SWAB.1.5.6 | DETECTED | 20.88 (24.18) | POSITIVE |
| 7 | SWAB.1.5.7 | NOT DETECTED | — | NEGATIVE |
| 8 | SWAB.1.5.8 | DETECTED | 20.96 (24.26) | POSITIVE |
| 9 | SWAB.1.5.9 | DETECTED | 32.16 (35.36) | NEGATIVE |
| | | | | (False negative) |
| 10 | SWAB.1.5.10 | DETECTED | 29.75 (33.05) | POSITIVE |

RT-LAMP 20 May 2020

| | | | | |
|---|---|---|---|---|
| 11 | SWAB.20.5.1 | DETECTED | 19.18 (22.48) | POSITIVE |
| 12 | SWAB.20.5.2 | DETECTED | 17.53 (20.83) | POSITIVE |
| 13 | SWAB.20.5.3 | DETECTED | 21.64 (24.94) | POSITIVE |
| 14 | SWAB.20.5.4 | DETECTED | 22.61 (25.91) | POSITIVE |
| 15 | SWAB.20.5.5 | DETECTED | 25.25 (28.55) | POSITIVE |
| 16 | SWAB.20.5.6 | DETECTED | 34.07 (37.37) | NEGATIVE |
| | | | | (False negative) |
| 17 | SWAB.20.5.7 | NOT DETECTED | — | NEGATIVE |
| 18 | SWAB.20.5.8 | NOT DETECTED | — | NEGATIVE |
| 19 | SWAB.20.5.9 | DETECTED | 28.07 (31.37) | NEGATIVE |
| | | | | (False negative) |
| 20 | SWAB.20.5.10 | NOT DETECTED | — | NEGATIVE |
| 21 | SWAB.20.5.11 | NOT DETECTED | — | NEGATIVE |
| 22 | SWAB.20.5.12 | NOT DETECTED | — | NEGATIVE |
| 23 | SWAB.20.5.13 | NOT DETECTED | — | NEGATIVE |
| 24 | SWAB.20.5.14 | NOT DETECTED | — | NEGATIVE |
| 25 | SWAB.20.5.15 | NOT DETECTED | — | NEGATIVE |

RT-LAMP 3 Jun. 2020

| | | | | |
|---|---|---|---|---|
| 26 | SWAB.3.6.1 | NOT DETECTED | — | NEGATIVE |
| 27 | SWAB.3.6.2 | NOT DETECTED | — | NEGATIVE |
| 28 | SWAB.3.6.3 | NOT DETECTED | — | NEGATIVE |
| 29 | SWAB.3.6.4 | NOT DETECTED | — | NEGATIVE |
| 30 | SWAB.3.6.5 | NOT DETECTED | — | NEGATIVE |
| 31 | SWAB.3.6.6 | NOT DETECTED | — | NEGATIVE |
| 32 | SWAB.3.6.7 | NOT DETECTED | — | NEGATIVE |
| 33 | SWAB.3.6.8 | NOT DETECTED | — | NEGATIVE |
| 34 | SWAB.3.6.9 | DETECTED | 13.89 (17.19) | POSITIVE |
| 35 | SWAB.3.6.10 | NOT DETECTED | — | NEGATIVE |
| 36 | SWAB.3.6.11 | DETECTED | 17.51 (20.45) | POSITIVE |
| 37 | SWAB.3.6.12 | NOT DETECTED | — | NEGATIVE |

RT-LAMP 11 Jun. 2020

| | | | | |
|---|---|---|---|---|
| 38 | SWAB.11.6.1 | DETECTED | 24.27 (27.57) | POSITIVE |
| 39 | SWAB.11.6.2 | NOT DETECTED | — | NEGATIVE |
| 40 | SWAB.11.6.3 | NOT DETECTED | — | NEGATIVE |
| 41 | SWAB.11.6.4 | NOT DETECTED | — | NEGATIVE |
| 42 | SWAB.11.6.5 | NOT DETECTED | — | NEGATIVE |
| 43 | SWAB.11.6.6 | DETECTED | 29.41 (32.71) | NEGATIVE |
| | | | | (False negative) |
| 44 | SWAB.11.6.7 | NOT DETECTED | — | NEGATIVE |
| 45 | SWAB.11.6.8 | NOT DETECTED | — | NEGATIVE |
| 46 | SWAB.11.6.9 | NOT DETECTED | — | NEGATIVE |
| 47 | SWAB.11.6.10 | NOT DETECTED | — | NEGATIVE |

* Ct value (equivalent Ct value account for 10× dilution effect)²⁰.

TABLE 5
Summary of Clinical testing results.

| | Clinical RNA testing | Direct swab testing |
|---|---|---|
| Total samples | 72 | 47 |
| | (16 Positive + 56 | (20 Positive + 27 |
| | Negative) | Negative) |
| Positive | 87.5% | 75.0% |
| successful rate | | |
| Negative | 91.1% | 100% |
| successful rate | | |
| Detection time | 15-30 min | 30-60 min |
| Limit of Detection | 50 copies/reaction | 100 copies/reaction* |

*Equivalent to Ct = 35 using PHE RdRp Assay

### Example 7 Use of Two Sets of Primers Enhanced Detection Sensitivity of SARS-CoV-2

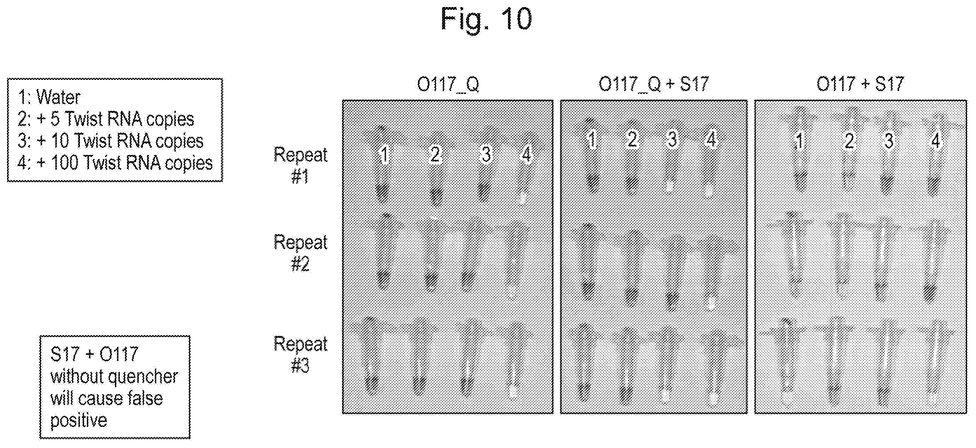

RT-LAMP was carried out using two sets of primers, O117_Q and S17 (Huang et al 2020). Sensitivity was significantly enhanced compared to use of O117_Q primers alone (FIG. 10). The switch in O117_Q also prevented false positive using these two sets of primers (FIG. 10).

### DISCUSSION

The rapid spread of SARS-CoV-2, including to resource limited areas, requests the development of novel, rapid methods for detecting infection in individuals. The standard diagnostic tool, RT-qPCR, cannot be readily deployed outside of large diagnostic laboratories due to the necessary technical expertise, sophisticated instrumentation, and costly reagents required for samples preparation and processing. The RT-LAMP assay is an ideal POCT as the only required equipment is a simple, inexpensive heating platform, which could be a heating block, dry incubator, water bath or thermal cycler. The assay is simple to perform, delivering an accurate result rapidly that can be easily interpreted by several different detection systems including visual, fluorescence and absorbance detection, but also by simple visual inspection in a POC setting.

### A Short Oligo Switch to Minimise False Positives

We have introduced several adaptations to improve on the performance of our previously reported RT-LAMP assay that differentiate us from other published RT-LAMP assays for SARS-CoV-2 detection. To minimize the risk of false positive due to unspecific amplification of dimers in RT-LAMP, we introduce a short oligo switch with sequence complementary to the FIP primer, which ‘locks’ FIP from random self-amplification in the absence of the target RNA. In the presence of the target RNA sequence of SARS-CoV-2, the switch is competitively off-binding the FIP primer and the RT-LAMP reaction proceeds. The addition of the switch decreased the risk of self- or off-target amplification (FIG. 1) and did not demonstrably impair the sensitivity of the assay (FIG. 6a).

Detection Limit of this RT-LAMP Assay

We have demonstrated that the RT-LAMP assay is sensitive and highly specific for SARS-CoV-2 RNA. Neither O117_N nor O117_Q primer set produced false positive results over eight reactions of samples containing the four seasonal coronaviruses. The 50% endpoint values for transcripts 19/304 and RNA control 2 are largely in agreement and demonstrate that the RT-LAMP assay can readily detect <100 copies of SARS-CoV-2 RNA. The substantially higher 50% endpoint for RNA control 1 may be due to improper initial quantification during shipment of transcript degradation. While a trend towards increased sensitivity in O117_N as one may expect is noted, no statistically significant difference between the O117_N and O117_Q preparations was found when averaged across the 19/304 and RNA Control 2 transcripts, but this may be a consequence of the low sample size.

The RT-LAMP assay demonstrated high concordance with a standard RT-qPCR assay in a laboratory setting. RNA extracts from clinical samples were tested using the RT-LAMP assay and showed reasonably high levels of OPA (90.3%) compared to RT-qPCR. The false negative samples presented have Ct values greater than 34 (Table 3). Hence, it suggests that the detection limit of O117_Q is equivalent to Ct=34 for RT-qPCR assay.

The five false positives detected by our RT-LAMP assay from the RNA extract samples raise the concerns of self-amplification and carryover contamination repeatedly observed in RT-LAMP. We have sought to mitigate these risks through the addition of the switch oligonucleotide which demonstrably increases the stability of the reaction. Through vacuum-drying the kit components, we also simplify the process for end users and limit the opportunities for onsite carryover contamination.

The ability to detect SARS-CoV-2 virus without the need for laborious and time-consuming RNA extraction is attractive. The RT-LAMP assay using direct swab samples was shown to be highly robust at detecting positive samples—demonstrating no false positive when comparing to RT-qPCR results. Since all swab samples were diluted 10 fold before the detection. Such 10 times dilution is equivalent to 3.3 Ct, according to the calibration between copy number of Orf1ab gene (O117_Q target gene) and Ct value in RT-qPCR (Niu et al. (2020)). If we consider the dilution effect and detection limit equivalent to Ct=34, the RT-LAMP assay missed two swab samples (Ct=31.37 and 32.71), and the positive percentage agreement (PPA) can be recalculated as 88% (15/17 SARS-CoV-2 positive samples) and an OPA of 95% (42/44 matching samples) (Table 4). These results were similar to that found in clinical RNA extracts, demonstrating the validity of using oro-nasopharyngeal swabs in place of extracted RNA without compromising assay performance. Since saline will inhibit enzymes in RT-LAMP assay and conventional universal transport medium will interfere colorimetric reading, one way to improve direct swab assay may be to put swab in RNase-free water. This slight modification in swab sampling for RT-LAMP assay could potentially detect swab samples with higher Ct value if the dilution factor is reduced (Niu et al. (2020)).

We found that two sets of primers including O117_Q and S17 can significantly enhance sensitivity (FIG. 10). The switch in O117_Q also prevents false positive using these two sets of primers.

### Drying Mixes Enables Room Temperature Transport and Storage

Drying the master mix simplifies the process for the end user—all that is required is the addition of water and the patient sample. A further advantage of the dried reagent form is its long-term durability and storage at room temperature. Performance of the dried O117_N and O117_Q kits was not compromised by storage at room temperature for 14 days, although the color of the kits might change to yellow after 5 days due to the change of pH. However, pH and the colour of the kits can be re-adjusted to the original pink colour by adding 2.5 μl of 10 mM KOH. The pH corrected kits still worked as normal in response to water control and full viral RNA transcript (FIG. 3). A fluorescence quantitative reading showed that the dry kits responded well to viral RNA after the dry kits were stored at room temperature for 14 days (FIG. 4), suggesting that Bst 2.0 DNA polymerase and WarmStart reverse transcriptase in NEB mastermix remain active for a long duration under drying storage condition. The dried kits can be transported without cold chain constraints, reducing transport and storage costs, increasing the resilience of the supply chain and providing access to testing for resource limited settings and POCT outside of standard diagnostic laboratories.

### Colorimetric and Fluorescent Double Display to Report RT-LAMP Results

Colorimetric RT-LAMP detection methods rely on the qualitative assessment of a pH-dependent colour change of phenol red as an indirect indication of polymerase amplification of the target sequence. As visual interpretation of the experimental results could be subjective, we have established three different quantitative methods to overcome these limitations. The first method exploits the absorbance changes associated with phenol red as a function of pH and the other two methods utilized fluorescent dyes and functions independent of pH. The fluorescent methods are particularly attractive as they function independent to the pH changes and could potentially overcome the problem associated with buffers present in the sample collection media affecting the readouts associated with phenol red. Although LAMP reactions have been conducted in a qPCR machine with real time fluorescence, this necessitates the use of a qPCR machine. Here we show that these readouts can be performed accurately using relatively inexpensive equipment that is readily available to many laboratories. Furthermore, these methods provide additional flexibility to the end users such that the quantification methods can be chosen according to equipment availability and/or throughput requirements. Where higher throughput is required, the RT-LAMP reaction can be conducted in a multi-well format optimised for 96 or 384 well plates using a fluorescent dye such as Syto9 and read with a microplate reader. On the other hand, the Qubit 2.0 is a relatively inexpensive desktop fluorometer requiring only a power source and can be readily integrated into the testing pipelines in a variety of diverse settings, including GP clinics and airports.

Thus, given the high accuracy of our LAMP assay, the potential use of multiple detection systems (including portable options such as Qubit), and ease of assay performance, we believe this adaptable set-up could be used as an effective and highly practical first-line screening tool and should undergo further clinical studies.

### Methods

### Primer Design.

Primers were designed as previously described in Huang et al. (2020). The primer set O117 targeting Orf1ab of SARS-CoV-2 was designed using PrimerExplorer (http://primerexplorer.jp/e/and Tomita et al. (2008)) and each primer was synthesised by Integrated DNA Technologies (IDT, UK). The Switch was synthesised by Integrated DNA Technologies (IDT, UK) with an Iowa Black Dark Quencher at the 3′ end (See Table 3).

10× O117_N primer mix was prepared by mixing equal volumes of 16 μM FIP, 16 μM BIP, 2 μM F3, 2 μM B3, 4 μM LF and 4 μM LB.

10× O117_Q primer mix was prepared by mixing equal volumes of 16 μM FIP, 16 μM BIP, 2 μM F3, 2 μM B3, 4 μM LF, 4 μM LB and 24 μM Switch.

### RT-LAMP Reaction Mix Preparation and Generalized Procedure.

All equipment, laminar-flow cabinets, and working benches were sprayed with 70% w/v ethanol (Sigma-Aldrich) and RNaseZap™ (Sigma-Aldrich) prior to reaction mix preparation. All reagents and PCR tubes were kept on ice during kit preparation.

For wet reaction mix, 12.5 μL of WarmStart™ Colorimetric LAMP 2× Master Mix (DNA & RNA) from New England Biolabs (NEB), 5 μL of DNase & RNase-free molecular grade water (Thermo Fisher Scientific), and 2.5 μL of 10× O117_N or 10× O117_Q primer mix was added into PCR tubes and mixed homogeneously.

For dried reaction mix, 12.5 μL 2× Master Mix was supplemented with 4 μL drying protective agent, OSD-D1™ (Oxsed, Oxford) containing bovine serum albumin, dextran, lactose and trehalose. 2.5 μL of 10× O117_N or 10× O117_Q primer mix was then added into the PCR tubes. The PCR tubes with caps open were dried with a concentrator (Concentrator Plus, Eppendorf). After desiccated, the caps of the PCR tubes were closed immediately and the dried reaction mixes were stored at −20° C.

The RT-LAMP reaction was run by adding 5 μL of sample of interest to a 20 μL wet reaction mix or a dried reaction mix resuspended in 20 μL of DNase/RNase-free water and heating the reaction at 65° C. for 30 minutes. A positive result is confirmed through a pH-dependent colorimetric change or confirmation of the production of LAMP product.

### Full-Length Transcripts.

Three full-length SARS-CoV-2 RNA transcripts were used in this study. Research reagent 19/304 (NIBSC) was the kind gift of Giada Mattiuzo. Synthetic RNA Control 1—MT007544.1 (Twist Bioscience) and Synthetic RNA Control 2—MN908947.3 (Twist Bioscience) were the kind gifts of John Taylor (Oxford Medical Genetics Laboratories).

### Clinical Samples.

Briefly, oro-nasopharyngeal swabs were stored in Universal Transfer Medium (COPAN Diagnostic, USA) or 0.85% saline and extracted using the QIAsymphony system (Qiagen, Hilden Germany). RNA was eluted in buffer AVE. Following extraction, 5 μL of the sample was carried into a RT-qPCR amplification using the Rotor-Gene Q (Qiagen). RT-qPCR reactions were set up using an RNA-dependent RNA polymerase (RdRP) gene target validated by Public Health England (PHE) and the RealStar SARS-CoV-2 RT-PCR Kit targeting the E and S genes. 5 μL of the eluted RNA sample was taken into a wet reaction mix containing O117_Q for RT-LAMP assay.

Swabs for direct processing were transported in 0.85% saline solution and processed within 48 hours in a Biosafety Level 3 lab. 50 μL of sample in saline solution was transferred into 450 μL of DNase/RNase-free water, mixed well, and 25 μL of diluted sample was taken directly into dried reaction mix containing O117_Q for RT-LAMP assay.

### Analytic Stability, Sensitivity, and Specificity.

The stability of the wet reaction mix containing O117_N or O117_Q was assessed. 5 μL of 40 copies/μL of Synthetic RNA Control 2 was added as a positive control and 5 μL of 0.2 μg/μL human genome cDNA was added as a negative control. Human genome cDNA was reverse transcribed from whole RNA extracted from human bone marrow derived MSC line (Lonza, UK). RNA extraction was performed as previously described (Brinkhof et al. (2018) and Brinkhof et al. (2006)) and reverse transcribed using the QuantiTect Reverse Transcription kit (Qiagen, Manchester, UK). 0.5-1 μg whole RNA was converted into cDNA per manufacturer's instructions including a genomic DNA wipe-out step. Final cDNA sample was diluted to 0.2 μg/μL in RNAse/DNAse free water and stored at −20° C. until used for RT-LAMP assay as negative control. The reaction was run as described above and the colour change was assessed at 0, 30, and 60 minutes.

To obtain an estimate of analytic sensitivity, the 50% endpoint of the LAMP assay was assessed using the three full-length SARS-CoV-2 transcripts. 19/304 was extracted using the viral RNA mini kit (Qiagen) and eluted in buffer AVE. Serial dilutions of 5 μL of RNA in buffer AVE were used to spike 20 μL of solution from a 50 μL negative throat swab resuspended in 450 μL of RNAse-free water. 25 μL of solution was added to a dried kit containing O117_N or O117_Q primers and were tested in parallel and with five replicates. The LAMP assay was run as described above and assessed via colour change and confirmed via UV-Vis of LAMP product on a 2% agarose gel stained with Sybr-Safe (Thermofisher). The 50% endpoint was calculated using the Reed-Muench method (Reed and Muench (1938)).

The specificity of the LAMP assay for SARS-CoV-2 RNA against other human-infective coronaviruses was assessed. RNA was extracted from biobanked respiratory samples positive for OC43, HKU1, NL63, and 229E (matched samples were previously collected and processed as described in Gaunt et al. (2010) using the QIAamp Viral RNA Mini Kit (Qiagen) according to the manufacturer's instructions. 20 μL of solution from a 50 μL negative throat swab resuspended in 450 μL of RNAse-free water was spiked with 5 μL of the RNA eluate. 25 μL was added to a dried kit containing O117_N or O117_Q primers and were tested in parallel. The LAMP assay was run in duplicate at 65° C. for 40 minutes and assessed via colour change.

### Quantitative Detection Methods.

Three methods for converting the qualitative colour change output of RT-LAMP reactions into a quantitative output were assessed. The absorbance spectrum of negative and positive samples was read using a BMG Labtech Fluorostar Omega microplate reader, and the reaction outcome was assessed using the ratio of 430/560 nm. Detection of the LAMP product was also performed using an intercalating fluorescent dye, Syto-9, at a final concentration of 3 μM (Thermofisher), added to the reaction mix once the 30 minutes RT-LAMP reaction was completed. Excitation and emission were performed at 485 nm and 500 nm, respectively (BMG Labtech Fluorostar Omega microplate reader). Alternatively, 5 μL of the reaction product was carried into the Qubit dsDNA BR Assay Kit (Invitrogen). The assay was performed according to the manufacturer's instructions and read using a Qubit 2.0 fluorometer. Cutoff for positive results for absorbance and Syto-9 quantitative assays were determined to be 3× standard deviations above the negative controls. For Qubit, 3× standard deviation was calculated using negative samples and negative controls.

### Example 8—Temperature Dependent Oligo Switch Stabilises RT-LAMP Assay

Several reports have indicated that carryover contamination and off target amplification can result in false positives when conducting RT-LAMP assays (Baek et al. 2020, Thi et al. 2020, Hsieh et al. 2014, Kim et al. 2016). To address the problem of self- and off-target amplification, a short oligonucleotide designated as a switch was introduced, whose sequence is complementary to one of the primers used in the LAMP reaction and 3′-end was modified by a dark quencher molecule—Iowa Black RQ (IBRQ). The switch serves as a temperature-dependent switch to bind an essential primer of LAMP (e.g. FIP), limiting non-specific amplification and the formation of primer dimers. It is believed that when the temperature is below the working temperature of LAMP (e.g. 65° C.), the FIP primer is bound to the switch oligonucleotide. The switch likely prevents non-specific amplification of non-SARS-CoV-2 RNA eluted during sample processing by binding the FIP primer with higher affinity than contaminating nucleic acids. At the working temperature, the switch competes with the sample RNA for binding, allowing FIP to bind target RNA/DNA for reverse transcription and amplification of target nucleic acids.

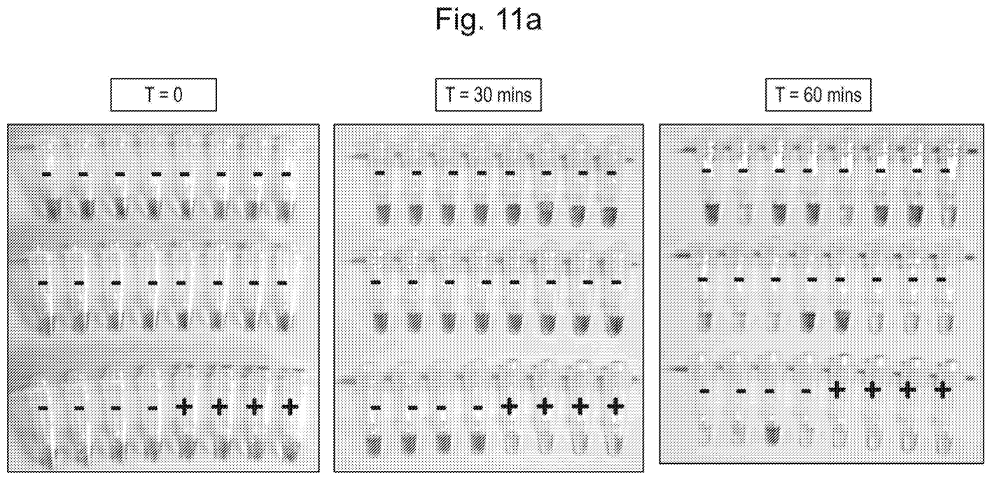

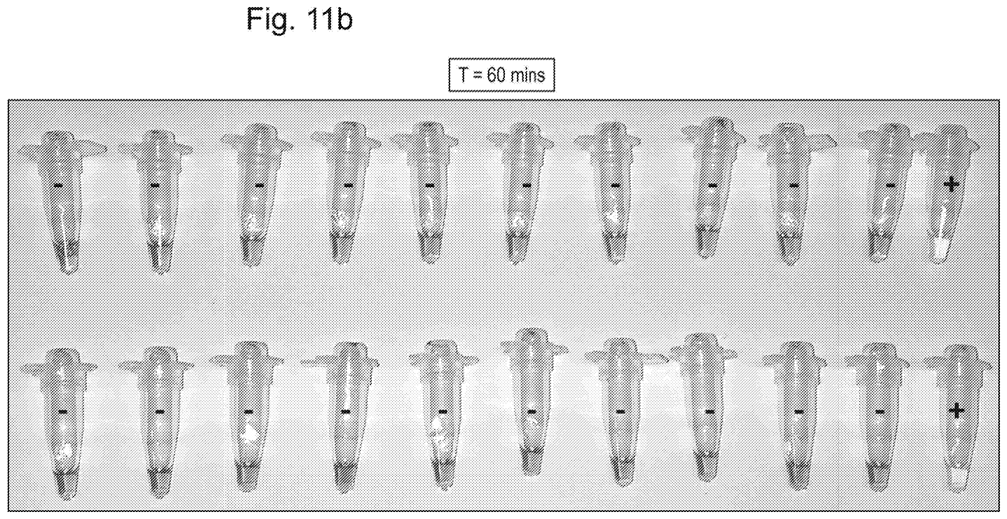

The O117 primer set was used to evaluate the performance of the switch. Two preparations of the primer mix were used throughout this study: O117_N contains the original six LAMP primers, and O117_Q contains the six LAMP primers and a switch oligonucleotide (Table 1). The blank controls without target template have been previously shown to result in false positive due to non-template amplification (Tanner et al. 2015). In this study, a 12-bp switch with 3′-end modification with IBRQ (IDT, UK) was designed to bind FIP and block self or off-target amplification when the temperature is below the RT-LAMP working temperature. O117_N or O117_Q primers were used to test Synthetic RNA Control 2—MN908947.3 (Twist Bioscience, US) and human cDNA (Sigma-Aldrich UK) through a prolonged incubation of up to 1 hour. WarmStart Colorimetric Master Mix (New England Biolabs, UK) was used for the RT-LAMP assay. After incubating at 65° C. for 30 minutes, both O117_N and O117_Q LAMP primers, showed positive (yellow) for 200 copies of Synthetic RNA Control 2, and negative (pink) for the human cDNA control (FIG. 1a). However, the O117_N primer mix turned yellow within one hour of heating, representing a false positive, while the O117_Q mix maintained a pink colour throughout the experiment (FIG. 1a). The O117_N primer set could generate up to 60% false positive result, whilst O117_Q can perform reliably after incubating at 65° C. for one hour (FIG. 11). The results suggest that a switch in O117 primers reduces off-target amplification at lower temperature and stabilises the LAMP assay at reaction temperature.

### Example 9—Freeze-Drying Improve Reaction Mix Storage and Transport

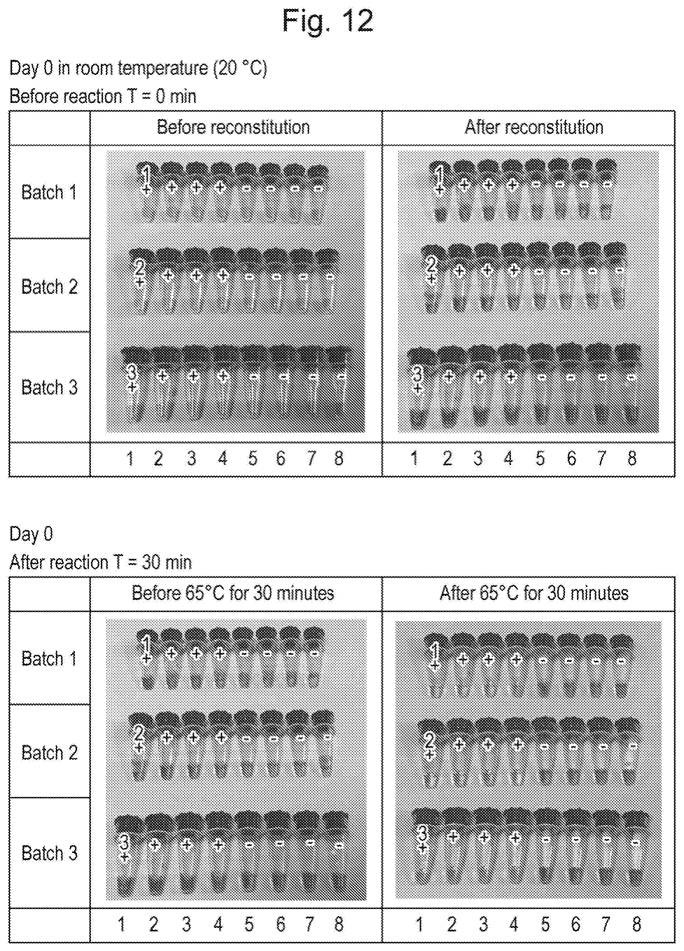

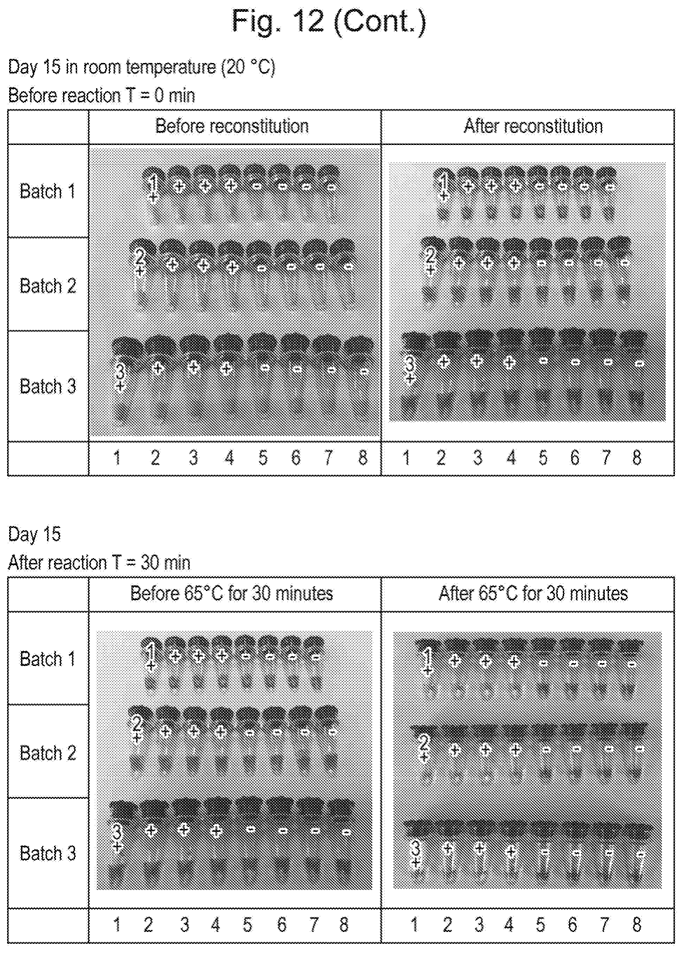

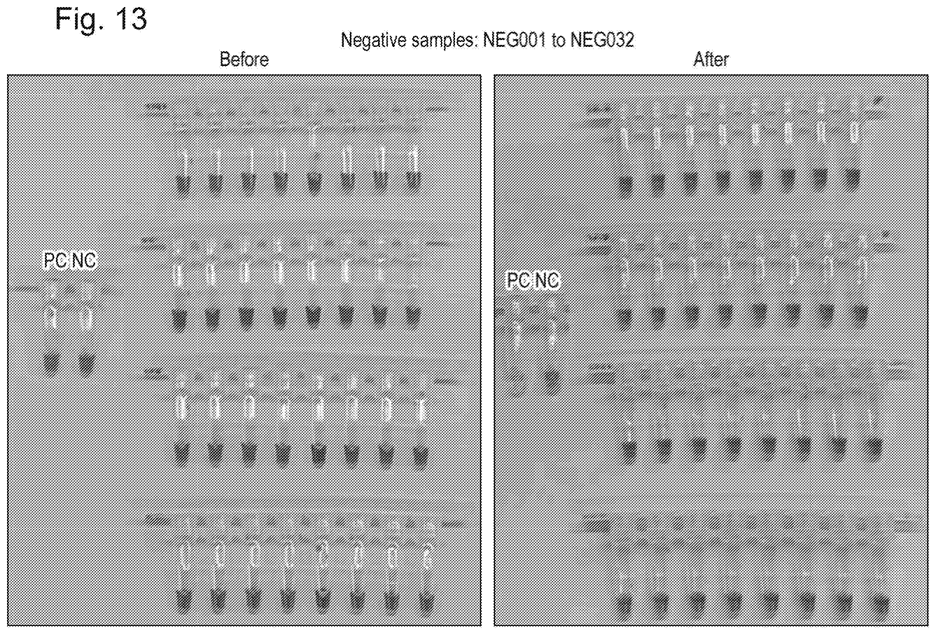

One limitation of the deployability of RT-LAMP to developing countries and rural areas is the need for cold-chain transport of reagents. To overcome this limitation, we freeze-dried a complete reagent mix, including Bst2.0 DNA polymerase, WarmStart reverse transcriptase, colorimetric indicator, dNTPs, primers, and buffer in one PCR tube to make a dry and ready-use reaction mixture. The stability of the dried reaction mix was assessed by storing at room temperature for 15 days before RT-LAMP assay was performed (FIG. 1b and FIG. 3 and FIG. 12). On day 3, the dried mixes were first re-suspended in 20 μL DNase/RNase free water followed by the addition of positive control (Synthetic RNA Control 2) and negative control (1 μg human genome cDNA) (FIG. 1b ii). After the RT-LAMP reaction, a clear colour difference was shown between the positive controls and negative controls, indicating a high retention of enzymatic activity after 3 days of dried storage at room temperature (FIG. 1b iii). The freeze-dried samples were more stable than vacuum-dried samples. Freeze-dried reaction mixes stored at room temperature for 15 days showed as good performance as day 0 mixes, indicating the dried kits can be stored for at least 15 days (FIG. 12).

### Example 10—Optimised RT-LAMP Reaction Mix Maintains High Sensitivity and Specificity

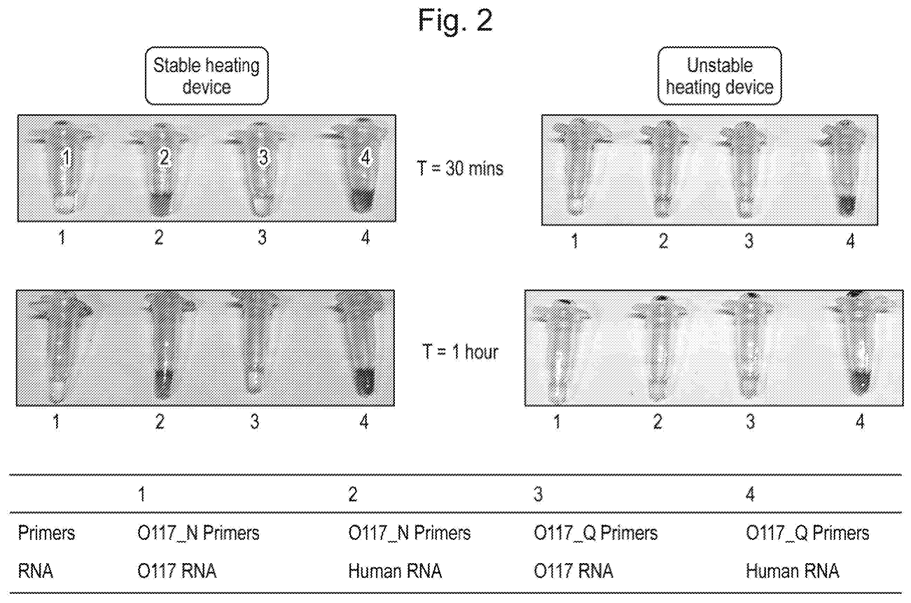

The 50% endpoints of detection (50EP) of dried kits of the RT-LAMP assay containing O117_N and O117_Q were determined using three full-length transcripts serially diluted in buffer AVE (Table 2). The RNA templates were research reagent 19/304 (NIBSC, UK), Synthetic RNA control 1—MT007544.1 and Synthetic RNA control 2 MN908947.3 (Twist BioScience, US). The testing results are summarised in Table 2, FIG. 2a and FIG. 5. It shows that 50EP against 19/304 were 71 (O117_Q) and 89 (O117_N) copies per reaction (25-μl). The 50EP of Synthetic RNA Control 1 was 131 and 224 copies per reaction for O117_Q and O117_N, respectively. Synthetic RNA Control 2 demonstrated 50EP of 60 and 13 copies per reaction for O117_Q and O117_N, respectively. Additionally, the 50EP was tested for detection of Synthetic RNA control 2 in wet reaction mixes. The level of detectable RNA for O117_Q and O117_N in wet reaction mix were 71 and 42 copies. The 50EP for dried reaction mixes containing O117_Q targeting 19/304 and Synthetic RNA Control 2 had a mean of 65 copies (95% CI: 57.3-72.9). The 50EP for dried reaction mixes containing O117_N and targeting 19/304 and Synthetic RNA control 2 had a mean of 51 copies (95% CI: −1.6-104.0). These results suggest that the oligo switch in O117 primers has a minimal impact on the LAMP sensitivity for viral RNA detection and the 50EP is 60-131 RNA copies per 25-μl reaction.

The ability of the optimised RT-LAMP assay to discriminate SARS-CoV-2 from human-infective seasonal coronaviruses was assessed using RNA extracted from stored clinical samples previously demonstrated to be infected with respiratory pathogens. The RNA samples from infected patient samples positive for betacoronaviruses (OC43 and HKU1) and alphacoronaviruses (229E and NL63) were used for specificity testing of O117_Q LAMP assay. Neither replicate of the seasonal coronaviruses tested by O117_Q produced a positive result, even using an extended reaction time (40 instead of 30 minutes) (FIGS. 2b and 2c). This result suggests that O117_Q primer set had a high specificity for SARS-CoV-2 and can discriminate other human-infective coronaviruses.

### Example 11—Quantitative Assessment of Colorimetric Readout

Reading the colorimetric RT-LAMP assay has an element of subjectivity. To overcome any ambiguity and potential for inter-user and inter-lab variation when using a visually-assessed colorimetric readout, quantitative measurements were established for differentiating positive and negative samples (FIG. 8). The first method uses the ratio of the absorbance at 430 nm and 560 nm to quantitatively assess the color change of the 72 swab samples (FIG. 7). The result is consistent with the colorimetric readout by the naked eye (FIGS. 8a and 8b). Fluorescent dyes SYTO 9 and Qubit were also used to detect the products of the RT-LAMP reaction. Specifically, RT-LAMP products were stained with SYTO 9 and read using a microplate reader (FIGS. 8c and 8d) or stained with Qubit BR DNA dye and read with a Qubit fluorometer (FIGS. 8e and 8f). Positive samples were defined as having values greater than three times the standard deviation of negative samples (n=18). As the Qubit is a portable, compact, and gives an accurate readout of fluorescence, we randomly selected a set of 10 positive and negative samples as identified previously using RT-qPCR to verify its applicability for POCT. Overall, all three methods demonstrated good agreement with the pH-dependent colorimetric RT-LAMP readout, validating their use as a quantitative measurement to indicate RT-LAMP outcomes. Specifically, absorbance, SYTO9, and Qubit revealed a 2.1, 2.1 and 9.4 fold change between SARS-CoV-2 positive and negative patient samples, respectively. Qubit reagents and fluorometer reading demonstrated a good performance.

### Example 12—Clinical Validation with RNA Extract from Patient Samples

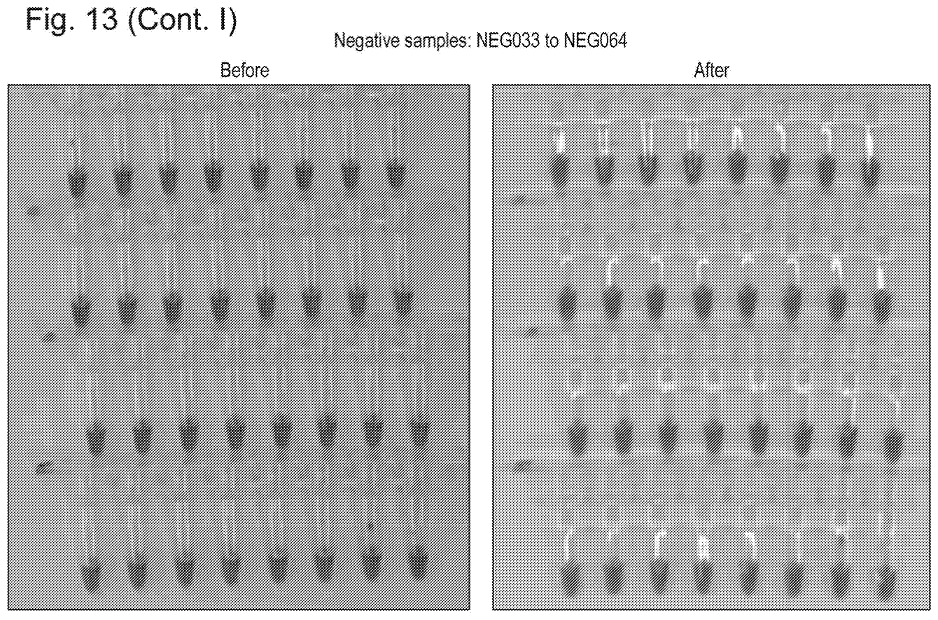

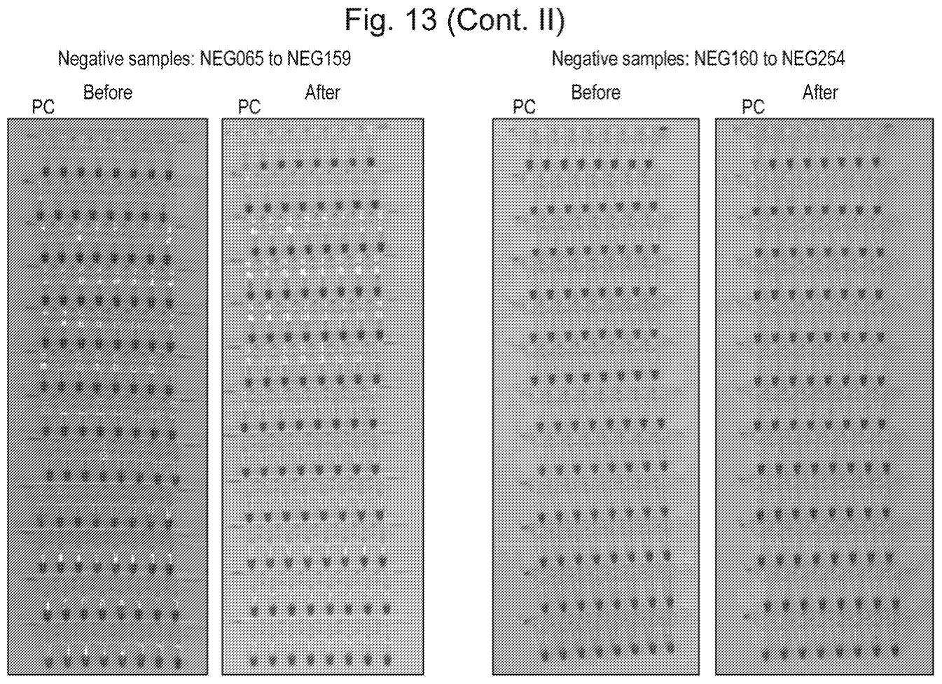

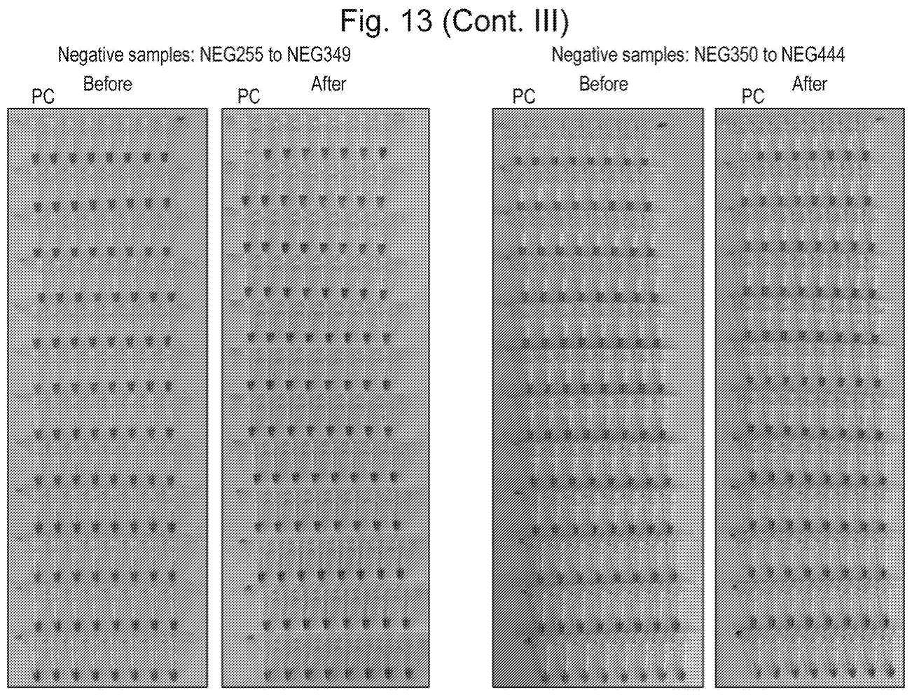

The performance of wet reaction mix containing O117_Q was evaluated using SARS-CoV-2 RNA extracts from 72 nasopharyngeal swab samples and 527 saliva samples. The clinical RNA samples were assessed by both RT-qPCR and RT-LAMP in parallel and the results were shown in Tables 3 and 7. Negative controls and positive controls show the expected results (Table 7 and FIG. 13). Visually assessment of the pH-dependent color change was consistent with absorbance and fluorescent measurements (FIG. 8).

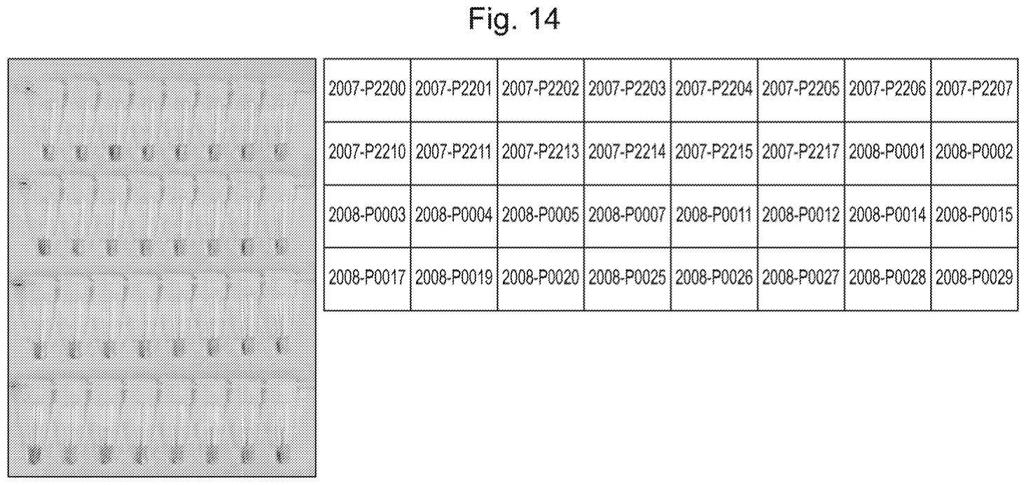

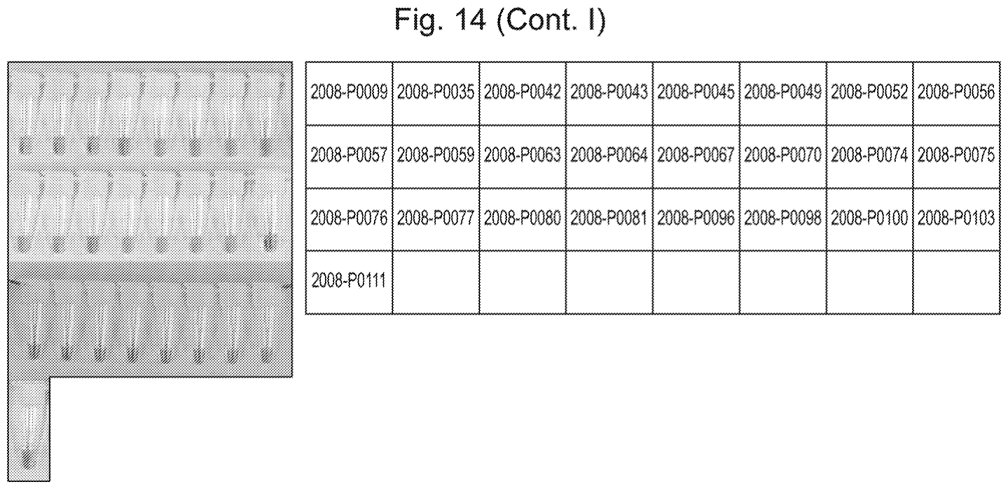

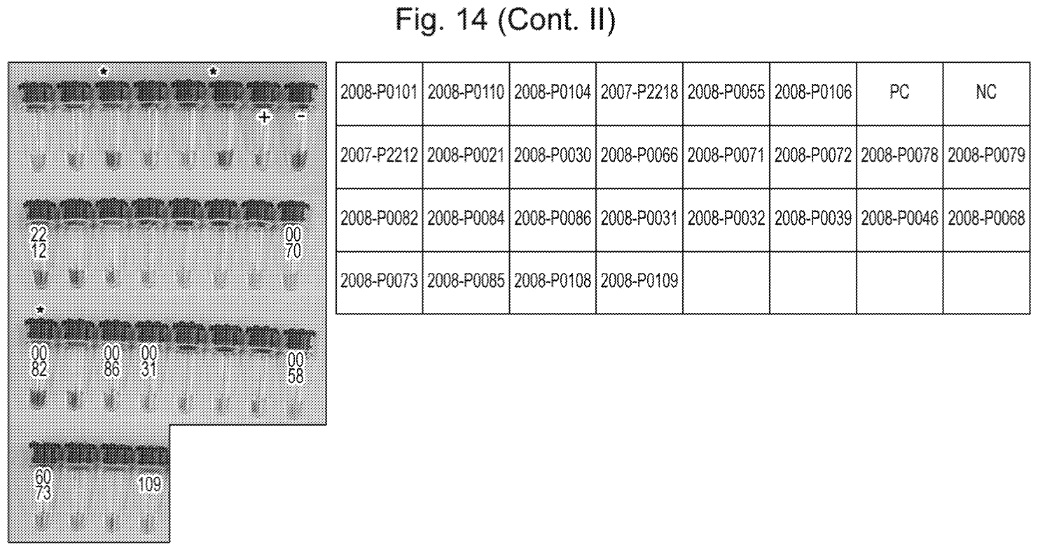

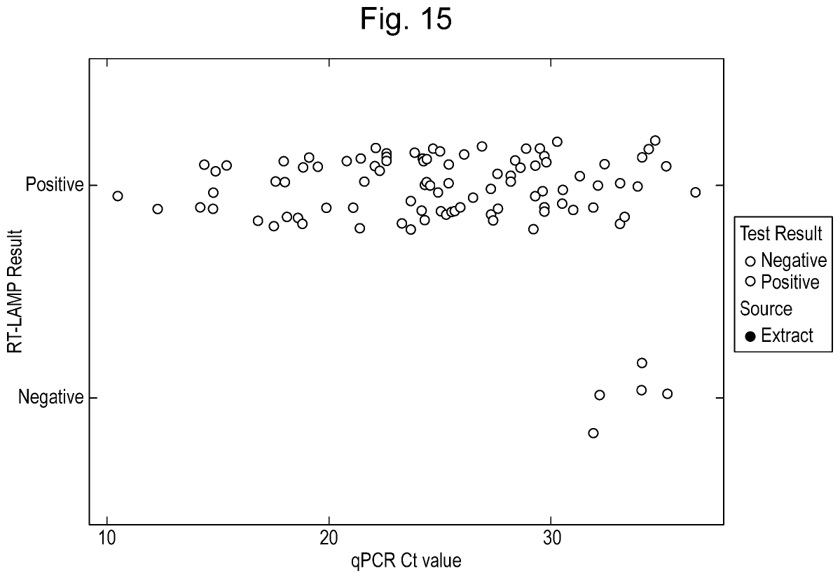

Compared to RT-qPCR, RT-LAMP demonstrated an overall sensitivity of 95% (SARS-CoV-2 samples; 95% CI: 88.6% to 98.3%) and specificity of 99% (95% CI: 97.7% to 99.7%) (Table 6). For the patient derived samples (elution buffer AVE, Qiagen UK), the sensitivity was 87.5% (95% CI: 61.6% to 98.4%) and specificity 91.1% (95% CI: 80.4% to 97.0%) (Table 6). For the restaurant staff samples (elution buffer Rnase free water), the sensitivity was 96.4% (95% CI: 89.8% to 99.2%) and specificity 100% (95% CI: 97.5%) (Table 6, Table 3, Table 7 and FIG. 14). The five false negative samples may have been due to the degradation of extracted RNA or the low SARS-CoV-2 copy numbers in the samples (FIG. 15).

TABLE 6
Summary of Clinical testing results.

| | Clinical RNA testing (Swab and Saliva) |
|---|---|
| Samples | 599 (72 swab and 527 saliva RNA extracts) |
| | Positive RT-PCR: 99 (16 swab + 83 saliva RNA extracts) |
| | Negative RT-PCR: 500 (56 swab + 444 saliva RNA extracts) |
| Overall | 95% (88.6-98.3) |
| sensitivity | |
| (95% CI) | |
| Overall | 99% (97.7-99.7) |
| specificity | |
| (95% CI) | |
| Detection | 15-30 min |
| time | |

TABLE 7
Performance of the improved RT-LAMP assay in detecting
SARS-COV-2 from RNA extract isolated from patient samples.
RT-LAMP Prenetics

| | | | RT-LAMP |
|---|---|---|---|
| Sample | RT-qPCR Result | Ct Value | Result |
| 2007-P2200 | DETECTED | 24.3 | Positive |
| 2007-P2201 | DETECTED | 27.3 | Positive |
| 2007-P2202 | DETECTED | 27.6 | Positive |
| 2007-P2203 | DETECTED | 20.8 | Positive |
| 2007-P2204 | DETECTED | 24.9 | Positive |
| 2007-P2205 | DETECTED | 19.9 | Positive |
| 2007-P2206 | DETECTED | 23.3 | Positive |
| 2007-P2207 | DETECTED | 19.5 | Positive |
| 2007-P2210 | DETECTED | 25.1 | Positive |
| 2007-P2211 | DETECTED | 24.7 | Positive |
| 2007-P2212 | DETECTED | 35.2 | Positive |
| 2007-P2213 | DETECTED | 18.1 | Positive |
| 2007-P2214 | DETECTED | 22.3 | Positive |
| 2007-P2215 | DETECTED | 14.9 | Positive |
| 2007-P2217 | DETECTED | 10.5 | Positive |
| 2007-P2218 | DETECTED | 32.1 | Positive |
| 2008-P0001 | DETECTED | 28.6 | Positive |
| 2008-P0002 | DETECTED | 14.4 | Positive |
| 2008-P0003 | DETECTED | 12.3 | Positive |
| 2008-P0004 | DETECTED | 23.9 | Positive |
| 2008-P0005 | DETECTED | 24.2 | Positive |
| 2008-P0007 | DETECTED | 24.3 | Positive |
| 2008-P0009 | DETECTED | 21.1 | Positive |
| 2008-P0011 | DETECTED | 24.2 | Positive |
| 2008-P0012 | DETECTED | 25.3 | Positive |
| 2008-P0014 | DETECTED | 21.6 | Positive |
| 2008-P0015 | DETECTED | 18.6 | Positive |
| 2008-P0017 | DETECTED | 29.3 | Positive |
| 2008-P0019 | DETECTED | 18.8 | Positive |
| 2008-P0020 | DETECTED | 29.5 | Positive |
| 2008-P0021 | DETECTED | 34.4 | Positive |
| 2008-P0025 | DETECTED | 27.3 | Positive |
| 2008-P0026 | DETECTED | 25.6 | Positive |
| 2008-P0027 | DETECTED | 15.4 | Positive |
| 2008-P0028 | DETECTED | 14.8 | Positive |
| 2008-P0029 | DETECTED | 26.5 | Positive |
| 2008-P0030 | DETECTED | 30.3 | Positive |
| 2008-P0031 | DETECTED | 29.2 | Positive |
| 2008-P0032 | DETECTED | 28.4 | Positive |
| 2008-P0035 | DETECTED | 25.7 | Positive |
| 2008-P0039 | DETECTED | 29.6 | Positive |
| 2008-P0042 | DETECTED | 21.4 | Positive |
| 2008-P0043 | DETECTED | 25.4 | Positive |
| 2008-P0045 | DETECTED | 18.0 | Positive |
| 2008-P0046 | DETECTED | 29.7 | Positive |
| 2008-P0049 | DETECTED | 28.9 | Positive |
| 2008-P0052 | DETECTED | 25.4 | Positive |
| 2008-P0055 | DETECTED | 33.1 | Positive |
| 2008-P0056 | DETECTED | 29.7 | Positive |
| 2008-P0057 | DETECTED | 24.4 | Positive |
| 2008-P0059 | DETECTED | 14.8 | Positive |
| 2008-P0063 | DETECTED | 34.7 | Positive |
| 2008-P0064 | DETECTED | 22.6 | Positive |
| 2008-P0066 | DETECTED | 33.3 | Positive |
| 2008-P0067 | DETECTED | 19.1 | Positive |
| 2008-P0068 | DETECTED | 28.2 | Positive |
| 2008-P0070 | DETECTED | 22.6 | Positive |
| 2008-P0071 | DETECTED | 34.1 | Positive |
| 2008-P0072 | DETECTED | 31.0 | Positive |
| 2008-P0073 | DETECTED | 29.8 | Positive |
| 2008-P0074 | DETECTED | 26.9 | Positive |
| 2008-P0075 | DETECTED | 21.1 | Positive |
| 2008-P0076 | DETECTED | 25.5 | Positive |
| 2008-P0077 | DETECTED | 23.7 | Positive |
| 2008-P0078 | DETECTED | 33.9 | Positive |
| 2008-P0079 | DETECTED | 32.4 | Positive |
| 2008-P0080 | DETECTED | 24.4 | Positive |
| 2008-P0081 | DETECTED | 36.5 | Positive |
| 2008-P0082 | DETECTED | 32.2 | Negative |
| | | | (False |
| | | | negative) |
| 2008-P0084 | DETECTED | 33.1 | Positive |
| 2008-P0085 | DETECTED | 27.6 | Positive |
| 2008-P0086 | DETECTED | 31.9 | Positive |
| 2008-P0096 | DETECTED | 26.1 | Positive |
| 2008-P0098 | DETECTED | 17.6 | Positive |
| 2008-P0100 | DETECTED | 29.7 | Positive |
| 2008-P0101 | DETECTED | 30.5 | Positive |
| 2008-P0103 | DETECTED | 14.2 | Positive |
| 2008-P0104 | DETECTED | 31.9 | Negative |
| | | | (False |
| | | | negative) |
| 2008-P0106 | DETECTED | 34.1 | Negative |
| | | | (False |
| | | | negative) |
| 2008-P0108 | DETECTED | 27.4 | Positive |
| 2008-P0109 | DETECTED | 25.9 | Positive |
| 2008-P0110 | DETECTED | 31.3 | Positive |
| 2008-P0111 | DETECTED | 16.8 | Positive |
| NEG001 | NOT DETECTED | — | NEGATIVE |
| NEG002 | NOT DETECTED | — | NEGATIVE |
| NEG003 | NOT DETECTED | — | NEGATIVE |
| NEG004 | NOT DETECTED | — | NEGATIVE |
| NEG005 | NOT DETECTED | — | NEGATIVE |
| NEG006 | NOT DETECTED | — | NEGATIVE |
| NEG007 | NOT DETECTED | — | NEGATIVE |
| NEG008 | NOT DETECTED | — | NEGATIVE |
| NEG009 | NOT DETECTED | — | NEGATIVE |
| NEG010 | NOT DETECTED | — | NEGATIVE |
| NEG011 | NOT DETECTED | — | NEGATIVE |
| NEG012 | NOT DETECTED | — | NEGATIVE |
| NEG013 | NOT DETECTED | — | NEGATIVE |
| NEG014 | NOT DETECTED | — | NEGATIVE |
| NEG015 | NOT DETECTED | — | NEGATIVE |
| NEG016 | NOT DETECTED | — | NEGATIVE |
| NEG017 | NOT DETECTED | — | NEGATIVE |
| NEG018 | NOT DETECTED | — | NEGATIVE |
| NEG019 | NOT DETECTED | — | NEGATIVE |
| NEG020 | NOT DETECTED | — | NEGATIVE |
| NEG021 | NOT DETECTED | — | NEGATIVE |
| NEG022 | NOT DETECTED | — | NEGATIVE |
| NEG023 | NOT DETECTED | — | NEGATIVE |
| NEG024 | NOT DETECTED | — | NEGATIVE |
| NEG025 | NOT DETECTED | — | NEGATIVE |
| NEG026 | NOT DETECTED | — | NEGATIVE |
| NEG027 | NOT DETECTED | — | NEGATIVE |
| NEG028 | NOT DETECTED | — | NEGATIVE |
| NEG029 | NOT DETECTED | — | NEGATIVE |
| NEG030 | NOT DETECTED | — | NEGATIVE |
| NEG031 | NOT DETECTED | — | NEGATIVE |
| NEG032 | NOT DETECTED | — | NEGATIVE |
| NEG033 | NOT DETECTED | — | NEGATIVE |
| NEG034 | NOT DETECTED | — | NEGATIVE |
| NEG035 | NOT DETECTED | — | NEGATIVE |
| NEG036 | NOT DETECTED | — | NEGATIVE |
| NEG037 | NOT DETECTED | — | NEGATIVE |
| NEG038 | NOT DETECTED | — | NEGATIVE |
| NEG039 | NOT DETECTED | — | NEGATIVE |
| NEG040 | NOT DETECTED | — | NEGATIVE |
| NEG041 | NOT DETECTED | — | NEGATIVE |
| NEG042 | NOT DETECTED | — | NEGATIVE |
| NEG043 | NOT DETECTED | — | NEGATIVE |
| NEG044 | NOT DETECTED | — | NEGATIVE |
| NEG045 | NOT DETECTED | — | NEGATIVE |
| NEG046 | NOT DETECTED | — | NEGATIVE |
| NEG047 | NOT DETECTED | — | NEGATIVE |
| NEG048 | NOT DETECTED | — | NEGATIVE |
| NEG049 | NOT DETECTED | — | NEGATIVE |
| NEG050 | NOT DETECTED | — | NEGATIVE |
| NEG051 | NOT DETECTED | — | NEGATIVE |
| NEG052 | NOT DETECTED | — | NEGATIVE |
| NEG053 | NOT DETECTED | — | NEGATIVE |
| NEG054 | NOT DETECTED | — | NEGATIVE |
| NEG055 | NOT DETECTED | — | NEGATIVE |
| NEG056 | NOT DETECTED | — | NEGATIVE |
| NEG057 | NOT DETECTED | — | NEGATIVE |
| NEG058 | NOT DETECTED | — | NEGATIVE |
| NEG059 | NOT DETECTED | — | NEGATIVE |
| NEG060 | NOT DETECTED | — | NEGATIVE |
| NEG061 | NOT DETECTED | — | NEGATIVE |
| NEG062 | NOT DETECTED | — | NEGATIVE |
| NEG063 | NOT DETECTED | — | NEGATIVE |
| NEG064 | NOT DETECTED | — | NEGATIVE |
| NEG065 | NOT DETECTED | — | NEGATIVE |
| NEG066 | NOT DETECTED | — | NEGATIVE |
| NEG067 | NOT DETECTED | — | NEGATIVE |
| NEG068 | NOT DETECTED | — | NEGATIVE |
| NEG069 | NOT DETECTED | — | NEGATIVE |
| NEG070 | NOT DETECTED | — | NEGATIVE |
| NEG071 | NOT DETECTED | — | NEGATIVE |
| NEG072 | NOT DETECTED | — | NEGATIVE |
| NEG073 | NOT DETECTED | — | NEGATIVE |
| NEG074 | NOT DETECTED | — | NEGATIVE |
| NEG075 | NOT DETECTED | — | NEGATIVE |
| NEG076 | NOT DETECTED | — | NEGATIVE |
| NEG077 | NOT DETECTED | — | NEGATIVE |
| NEG078 | NOT DETECTED | — | NEGATIVE |
| NEG079 | NOT DETECTED | — | NEGATIVE |
| NEG080 | NOT DETECTED | — | NEGATIVE |
| NEG081 | NOT DETECTED | — | NEGATIVE |
| NEG082 | NOT DETECTED | — | NEGATIVE |
| NEG083 | NOT DETECTED | | NEGATIVE |
| NEG084 | NOT DETECTED | — | NEGATIVE |
| NEG085 | NOT DETECTED | — | NEGATIVE |
| NEG086 | NOT DETECTED | — | NEGATIVE |
| NEG087 | NOT DETECTED | — | NEGATIVE |
| NEG088 | NOT DETECTED | — | NEGATIVE |
| NEG089 | NOT DETECTED | — | NEGATIVE |
| NEG090 | NOT DETECTED | — | NEGATIVE |
| NEG091 | NOT DETECTED | — | NEGATIVE |
| NEG092 | NOT DETECTED | — | NEGATIVE |
| NEG093 | NOT DETECTED | — | NEGATIVE |
| NEG094 | NOT DETECTED | — | NEGATIVE |
| NEG095 | NOT DETECTED | — | NEGATIVE |
| NEG096 | NOT DETECTED | — | NEGATIVE |
| NEG097 | NOT DETECTED | — | NEGATIVE |
| NEG098 | NOT DETECTED | — | NEGATIVE |
| NEG099 | NOT DETECTED | — | NEGATIVE |
| NEG100 | NOT DETECTED | — | NEGATIVE |
| NEG101 | NOT DETECTED | — | NEGATIVE |
| NEG102 | NOT DETECTED | — | NEGATIVE |
| NEG103 | NOT DETECTED | — | NEGATIVE |
| NEG104 | NOT DETECTED | — | NEGATIVE |
| NEG105 | NOT DETECTED | — | NEGATIVE |
| NEG106 | NOT DETECTED | — | NEGATIVE |
| NEG107 | NOT DETECTED | — | NEGATIVE |
| NEG108 | NOT DETECTED | — | NEGATIVE |
| NEG109 | NOT DETECTED | — | NEGATIVE |
| NEG110 | NOT DETECTED | — | NEGATIVE |
| NEG111 | NOT DETECTED | — | NEGATIVE |
| NEG112 | NOT DETECTED | — | NEGATIVE |
| NEG113 | NOT DETECTED | — | NEGATIVE |
| NEG114 | NOT DETECTED | — | NEGATIVE |
| NEG115 | NOT DETECTED | — | NEGATIVE |
| NEG116 | NOT DETECTED | — | NEGATIVE |
| NEG117 | NOT DETECTED | — | NEGATIVE |
| NEG118 | NOT DETECTED | — | NEGATIVE |
| NEG119 | NOT DETECTED | — | NEGATIVE |
| NEG120 | NOT DETECTED | — | NEGATIVE |
| NEG121 | NOT DETECTED | — | NEGATIVE |
| NEG122 | NOT DETECTED | — | NEGATIVE |
| NEG123 | NOT DETECTED | — | NEGATIVE |
| NEG124 | NOT DETECTED | — | NEGATIVE |
| NEG125 | NOT DETECTED | — | NEGATIVE |
| NEG126 | NOT DETECTED | — | NEGATIVE |
| NEG127 | NOT DETECTED | — | NEGATIVE |
| NEG128 | NOT DETECTED | — | NEGATIVE |
| NEG129 | NOT DETECTED | — | NEGATIVE |
| NEG130 | NOT DETECTED | — | NEGATIVE |
| NEG131 | NOT DETECTED | — | NEGATIVE |
| NEG132 | NOT DETECTED | — | NEGATIVE |
| NEG133 | NOT DETECTED | — | NEGATIVE |
| NEG134 | NOT DETECTED | — | NEGATIVE |
| NEG135 | NOT DETECTED | — | NEGATIVE |
| NEG136 | NOT DETECTED | — | NEGATIVE |
| NEG137 | NOT DETECTED | — | NEGATIVE |
| NEG138 | NOT DETECTED | — | NEGATIVE |
| NEG139 | NOT DETECTED | — | NEGATIVE |
| NEG140 | NOT DETECTED | — | NEGATIVE |
| NEG141 | NOT DETECTED | — | NEGATIVE |
| NEG142 | NOT DETECTED | — | NEGATIVE |
| NEG143 | NOT DETECTED | — | NEGATIVE |
| NEG144 | NOT DETECTED | — | NEGATIVE |
| NEG145 | NOT DETECTED | — | NEGATIVE |
| NEG146 | NOT DETECTED | — | NEGATIVE |
| NEG147 | NOT DETECTED | — | NEGATIVE |
| NEG148 | NOT DETECTED | — | NEGATIVE |
| NEG149 | NOT DETECTED | — | NEGATIVE |
| NEG150 | NOT DETECTED | — | NEGATIVE |
| NEG151 | NOT DETECTED | — | NEGATIVE |
| NEG152 | NOT DETECTED | — | NEGATIVE |
| NEG153 | NOT DETECTED | — | NEGATIVE |
| NEG154 | NOT DETECTED | — | NEGATIVE |
| NEG155 | NOT DETECTED | — | NEGATIVE |
| NEG156 | NOT DETECTED | — | NEGATIVE |
| NEG157 | NOT DETECTED | — | NEGATIVE |
| NEG158 | NOT DETECTED | — | NEGATIVE |
| NEG159 | NOT DETECTED | — | NEGATIVE |
| NEG160 | NOT DETECTED | — | NEGATIVE |
| NEG161 | NOT DETECTED | — | NEGATIVE |
| NEG162 | NOT DETECTED | — | NEGATIVE |
| NEG163 | NOT DETECTED | — | NEGATIVE |
| NEG164 | NOT DETECTED | — | NEGATIVE |
| NEG165 | NOT DETECTED | — | NEGATIVE |
| NEG166 | NOT DETECTED | — | NEGATIVE |
| NEG167 | NOT DETECTED | — | NEGATIVE |
| NEG168 | NOT DETECTED | — | NEGATIVE |
| NEG169 | NOT DETECTED | — | NEGATIVE |
| NEG170 | NOT DETECTED | — | NEGATIVE |
| NEG171 | NOT DETECTED | — | NEGATIVE |
| NEG172 | NOT DETECTED | — | NEGATIVE |
| NEG173 | NOT DETECTED | — | NEGATIVE |
| NEG174 | NOT DETECTED | — | NEGATIVE |
| NEG175 | NOT DETECTED | — | NEGATIVE |
| NEG176 | NOT DETECTED | — | NEGATIVE |
| NEG177 | NOT DETECTED | — | NEGATIVE |
| NEG178 | NOT DETECTED | — | NEGATIVE |
| NEG179 | NOT DETECTED | — | NEGATIVE |
| NEG180 | NOT DETECTED | — | NEGATIVE |
| NEG181 | NOT DETECTED | — | NEGATIVE |
| NEG182 | NOT DETECTED | — | NEGATIVE |
| NEG183 | NOT DETECTED | — | NEGATIVE |
| NEG184 | NOT DETECTED | — | NEGATIVE |
| NEG185 | NOT DETECTED | — | NEGATIVE |
| NEG186 | NOT DETECTED | — | NEGATIVE |
| NEG187 | NOT DETECTED | — | NEGATIVE |
| NEG188 | NOT DETECTED | — | NEGATIVE |
| NEG189 | NOT DETECTED | — | NEGATIVE |
| NEG190 | NOT DETECTED | — | NEGATIVE |
| NEG191 | NOT DETECTED | — | NEGATIVE |
| NEG192 | NOT DETECTED | — | NEGATIVE |
| NEG193 | NOT DETECTED | — | NEGATIVE |
| NEG194 | NOT DETECTED | — | NEGATIVE |
| NEG195 | NOT DETECTED | — | NEGATIVE |
| NEG196 | NOT DETECTED | — | NEGATIVE |
| NEG197 | NOT DETECTED | — | NEGATIVE |
| NEG198 | NOT DETECTED | — | NEGATIVE |
| NEG199 | NOT DETECTED | — | NEGATIVE |
| NEG200 | NOT DETECTED | — | NEGATIVE |
| NEG201 | NOT DETECTED | — | NEGATIVE |
| NEG202 | NOT DETECTED | — | NEGATIVE |
| NEG203 | NOT DETECTED | — | NEGATIVE |
| NEG204 | NOT DETECTED | — | NEGATIVE |
| NEG205 | NOT DETECTED | — | NEGATIVE |
| NEG206 | NOT DETECTED | — | NEGATIVE |
| NEG207 | NOT DETECTED | — | NEGATIVE |
| NEG208 | NOT DETECTED | — | NEGATIVE |
| NEG209 | NOT DETECTED | — | NEGATIVE |
| NEG210 | NOT DETECTED | — | NEGATIVE |
| NEG211 | NOT DETECTED | — | NEGATIVE |
| NEG212 | NOT DETECTED | — | NEGATIVE |
| NEG213 | NOT DETECTED | — | NEGATIVE |
| NEG214 | NOT DETECTED | — | NEGATIVE |
| NEG215 | NOT DETECTED | — | NEGATIVE |
| NEG216 | NOT DETECTED | — | NEGATIVE |
| NEG217 | NOT DETECTED | — | NEGATIVE |
| NEG218 | NOT DETECTED | — | NEGATIVE |
| NEG219 | NOT DETECTED | — | NEGATIVE |
| NEG220 | NOT DETECTED | — | NEGATIVE |
| NEG221 | NOT DETECTED | — | NEGATIVE |
| NEG222 | NOT DETECTED | — | NEGATIVE |
| NEG223 | NOT DETECTED | — | NEGATIVE |
| NEG224 | NOT DETECTED | — | NEGATIVE |
| NEG225 | NOT DETECTED | — | NEGATIVE |
| NEG226 | NOT DETECTED | — | NEGATIVE |
| NEG227 | NOT DETECTED | — | NEGATIVE |
| NEG228 | NOT DETECTED | — | NEGATIVE |
| NEG229 | NOT DETECTED | — | NEGATIVE |
| NEG230 | NOT DETECTED | — | NEGATIVE |
| NEG231 | NOT DETECTED | — | NEGATIVE |
| NEG232 | NOT DETECTED | — | NEGATIVE |
| NEG233 | NOT DETECTED | — | NEGATIVE |
| NEG234 | NOT DETECTED | — | NEGATIVE |
| NEG235 | NOT DETECTED | — | NEGATIVE |
| NEG236 | NOT DETECTED | — | NEGATIVE |
| NEG237 | NOT DETECTED | — | NEGATIVE |
| NEG238 | NOT DETECTED | — | NEGATIVE |
| NEG239 | NOT DETECTED | — | NEGATIVE |
| NEG240 | NOT DETECTED | — | NEGATIVE |
| NEG241 | NOT DETECTED | — | NEGATIVE |
| NEG242 | NOT DETECTED | — | NEGATIVE |
| NEG243 | NOT DETECTED | — | NEGATIVE |
| NEG244 | NOT DETECTED | — | NEGATIVE |
| NEG245 | NOT DETECTED | — | NEGATIVE |
| NEG246 | NOT DETECTED | — | NEGATIVE |
| NEG247 | NOT DETECTED | — | NEGATIVE |
| NEG248 | NOT DETECTED | — | NEGATIVE |
| NEG249 | NOT DETECTED | — | NEGATIVE |
| NEG250 | NOT DETECTED | — | NEGATIVE |
| NEG251 | NOT DETECTED | — | NEGATIVE |
| NEG252 | NOT DETECTED | — | NEGATIVE |
| NEG253 | NOT DETECTED | — | NEGATIVE |
| NEG254 | NOT DETECTED | — | NEGATIVE |
| NEG255 | NOT DETECTED | — | NEGATIVE |
| NEG256 | NOT DETECTED | — | NEGATIVE |
| NEG257 | NOT DETECTED | — | NEGATIVE |
| NEG258 | NOT DETECTED | — | NEGATIVE |
| NEG259 | NOT DETECTED | — | NEGATIVE |
| NEG260 | NOT DETECTED | — | NEGATIVE |
| NEG261 | NOT DETECTED | — | NEGATIVE |
| NEG262 | NOT DETECTED | — | NEGATIVE |
| NEG263 | NOT DETECTED | — | NEGATIVE |
| NEG264 | NOT DETECTED | — | NEGATIVE |
| NEG265 | NOT DETECTED | — | NEGATIVE |
| NEG266 | NOT DETECTED | — | NEGATIVE |
| NEG267 | NOT DETECTED | — | NEGATIVE |
| NEG268 | NOT DETECTED | — | NEGATIVE |
| NEG269 | NOT DETECTED | — | NEGATIVE |
| NEG270 | NOT DETECTED | — | NEGATIVE |
| NEG271 | NOT DETECTED | — | NEGATIVE |
| NEG272 | NOT DETECTED | — | NEGATIVE |
| NEG273 | NOT DETECTED | — | NEGATIVE |
| NEG274 | NOT DETECTED | — | NEGATIVE |
| NEG275 | NOT DETECTED | — | NEGATIVE |
| NEG276 | NOT DETECTED | — | NEGATIVE |
| NEG277 | NOT DETECTED | — | NEGATIVE |
| NEG278 | NOT DETECTED | — | NEGATIVE |
| NEG279 | NOT DETECTED | — | NEGATIVE |
| NEG280 | NOT DETECTED | — | NEGATIVE |
| NEG281 | NOT DETECTED | — | NEGATIVE |
| NEG282 | NOT DETECTED | — | NEGATIVE |
| NEG283 | NOT DETECTED | — | NEGATIVE |
| NEG284 | NOT DETECTED | — | NEGATIVE |
| NEG285 | NOT DETECTED | — | NEGATIVE |
| NEG286 | NOT DETECTED | — | NEGATIVE |
| NEG287 | NOT DETECTED | — | NEGATIVE |
| NEG288 | NOT DETECTED | — | NEGATIVE |
| NEG289 | NOT DETECTED | — | NEGATIVE |
| NEG290 | NOT DETECTED | — | NEGATIVE |
| NEG291 | NOT DETECTED | — | NEGATIVE |
| NEG292 | NOT DETECTED | — | NEGATIVE |
| NEG293 | NOT DETECTED | — | NEGATIVE |
| NEG294 | NOT DETECTED | — | NEGATIVE |
| NEG295 | NOT DETECTED | — | NEGATIVE |
| NEG296 | NOT DETECTED | — | NEGATIVE |
| NEG297 | NOT DETECTED | — | NEGATIVE |
| NEG298 | NOT DETECTED | — | NEGATIVE |
| NEG299 | NOT DETECTED | — | NEGATIVE |
| NEG300 | NOT DETECTED | — | NEGATIVE |
| NEG301 | NOT DETECTED | — | NEGATIVE |
| NEG302 | NOT DETECTED | — | NEGATIVE |
| NEG303 | NOT DETECTED | — | NEGATIVE |
| NEG304 | NOT DETECTED | — | NEGATIVE |
| NEG305 | NOT DETECTED | — | NEGATIVE |
| NEG306 | NOT DETECTED | — | NEGATIVE |
| NEG307 | NOT DETECTED | — | NEGATIVE |
| NEG308 | NOT DETECTED | — | NEGATIVE |
| NEG309 | NOT DETECTED | — | NEGATIVE |
| NEG310 | NOT DETECTED | — | NEGATIVE |
| NEG311 | NOT DETECTED | — | NEGATIVE |
| NEG312 | NOT DETECTED | — | NEGATIVE |
| NEG313 | NOT DETECTED | — | NEGATIVE |
| NEG314 | NOT DETECTED | — | NEGATIVE |
| NEG315 | NOT DETECTED | — | NEGATIVE |
| NEG316 | NOT DETECTED | — | NEGATIVE |
| NEG317 | NOT DETECTED | — | NEGATIVE |
| NEG318 | NOT DETECTED | — | NEGATIVE |
| NEG319 | NOT DETECTED | — | NEGATIVE |
| NEG320 | NOT DETECTED | — | NEGATIVE |
| NEG321 | NOT DETECTED | — | NEGATIVE |
| NEG322 | NOT DETECTED | — | NEGATIVE |
| NEG323 | NOT DETECTED | — | NEGATIVE |
| NEG324 | NOT DETECTED | — | NEGATIVE |
| NEG325 | NOT DETECTED | — | NEGATIVE |
| NEG326 | NOT DETECTED | — | NEGATIVE |
| NEG327 | NOT DETECTED | — | NEGATIVE |
| NEG328 | NOT DETECTED | — | NEGATIVE |
| NEG329 | NOT DETECTED | — | NEGATIVE |
| NEG330 | NOT DETECTED | — | NEGATIVE |
| NEG331 | NOT DETECTED | — | NEGATIVE |
| NEG332 | NOT DETECTED | — | NEGATIVE |
| NEG333 | NOT DETECTED | — | NEGATIVE |
| NEG334 | NOT DETECTED | — | NEGATIVE |
| NEG335 | NOT DETECTED | — | NEGATIVE |
| NEG336 | NOT DETECTED | — | NEGATIVE |
| NEG337 | NOT DETECTED | — | NEGATIVE |
| NEG338 | NOT DETECTED | — | NEGATIVE |
| NEG339 | NOT DETECTED | — | NEGATIVE |
| NEG340 | NOT DETECTED | — | NEGATIVE |
| NEG341 | NOT DETECTED | — | NEGATIVE |
| NEG342 | NOT DETECTED | — | NEGATIVE |
| NEG343 | NOT DETECTED | — | NEGATIVE |
| NEG344 | NOT DETECTED | — | NEGATIVE |
| NEG345 | NOT DETECTED | — | NEGATIVE |
| NEG346 | NOT DETECTED | — | NEGATIVE |
| NEG347 | NOT DETECTED | — | NEGATIVE |
| NEG348 | NOT DETECTED | — | NEGATIVE |
| NEG349 | NOT DETECTED | — | NEGATIVE |
| NEG350 | NOT DETECTED | — | NEGATIVE |
| NEG351 | NOT DETECTED | — | NEGATIVE |
| NEG352 | NOT DETECTED | — | NEGATIVE |
| NEG353 | NOT DETECTED | — | NEGATIVE |
| NEG354 | NOT DETECTED | — | NEGATIVE |
| NEG355 | NOT DETECTED | — | NEGATIVE |
| NEG356 | NOT DETECTED | — | NEGATIVE |
| NEG357 | NOT DETECTED | — | NEGATIVE |
| NEG358 | NOT DETECTED | — | NEGATIVE |
| NEG359 | NOT DETECTED | — | NEGATIVE |
| NEG360 | NOT DETECTED | — | NEGATIVE |
| NEG361 | NOT DETECTED | — | NEGATIVE |
| NEG362 | NOT DETECTED | — | NEGATIVE |
| NEG363 | NOT DETECTED | — | NEGATIVE |
| NEG364 | NOT DETECTED | — | NEGATIVE |
| NEG365 | NOT DETECTED | — | NEGATIVE |
| NEG366 | NOT DETECTED | — | NEGATIVE |
| NEG367 | NOT DETECTED | — | NEGATIVE |
| NEG368 | NOT DETECTED | — | NEGATIVE |
| NEG369 | NOT DETECTED | — | NEGATIVE |
| NEG370 | NOT DETECTED | — | NEGATIVE |
| NEG371 | NOT DETECTED | — | NEGATIVE |
| NEG372 | NOT DETECTED | — | NEGATIVE |
| NEG373 | NOT DETECTED | — | NEGATIVE |
| NEG374 | NOT DETECTED | — | NEGATIVE |
| NEG375 | NOT DETECTED | — | NEGATIVE |
| NEG376 | NOT DETECTED | — | NEGATIVE |
| NEG377 | NOT DETECTED | — | NEGATIVE |
| NEG378 | NOT DETECTED | — | NEGATIVE |
| NEG379 | NOT DETECTED | — | NEGATIVE |
| NEG380 | NOT DETECTED | — | NEGATIVE |
| NEG381 | NOT DETECTED | — | NEGATIVE |
| NEG382 | NOT DETECTED | — | NEGATIVE |
| NEG383 | NOT DETECTED | — | NEGATIVE |
| NEG384 | NOT DETECTED | — | NEGATIVE |
| NEG385 | NOT DETECTED | — | NEGATIVE |
| NEG386 | NOT DETECTED | — | NEGATIVE |
| NEG387 | NOT DETECTED | — | NEGATIVE |
| NEG388 | NOT DETECTED | — | NEGATIVE |
| NEG389 | NOT DETECTED | — | NEGATIVE |
| NEG390 | NOT DETECTED | — | NEGATIVE |
| NEG391 | NOT DETECTED | — | NEGATIVE |
| NEG392 | NOT DETECTED | — | NEGATIVE |
| NEG393 | NOT DETECTED | — | NEGATIVE |
| NEG394 | NOT DETECTED | — | NEGATIVE |
| NEG395 | NOT DETECTED | — | NEGATIVE |
| NEG396 | NOT DETECTED | — | NEGATIVE |
| NEG397 | NOT DETECTED | — | NEGATIVE |
| NEG398 | NOT DETECTED | — | NEGATIVE |
| NEG399 | NOT DETECTED | — | NEGATIVE |
| NEG400 | NOT DETECTED | — | NEGATIVE |
| NEG401 | NOT DETECTED | — | NEGATIVE |
| NEG402 | NOT DETECTED | — | NEGATIVE |
| NEG403 | NOT DETECTED | — | NEGATIVE |
| NEG404 | NOT DETECTED | — | NEGATIVE |
| NEG405 | NOT DETECTED | — | NEGATIVE |
| NEG406 | NOT DETECTED | — | NEGATIVE |
| NEG407 | NOT DETECTED | — | NEGATIVE |
| NEG408 | NOT DETECTED | — | NEGATIVE |
| NEG409 | NOT DETECTED | — | NEGATIVE |
| NEG410 | NOT DETECTED | — | NEGATIVE |
| NEG411 | NOT DETECTED | — | NEGATIVE |
| NEG412 | NOT DETECTED | — | NEGATIVE |
| NEG413 | NOT DETECTED | — | NEGATIVE |
| NEG414 | NOT DETECTED | — | NEGATIVE |
| NEG415 | NOT DETECTED | — | NEGATIVE |
| NEG416 | NOT DETECTED | — | NEGATIVE |
| NEG417 | NOT DETECTED | — | NEGATIVE |
| NEG418 | NOT DETECTED | — | NEGATIVE |
| NEG419 | NOT DETECTED | — | NEGATIVE |
| NEG420 | NOT DETECTED | — | NEGATIVE |
| NEG421 | NOT DETECTED | — | NEGATIVE |
| NEG422 | NOT DETECTED | — | NEGATIVE |
| NEG423 | NOT DETECTED | — | NEGATIVE |
| NEG424 | NOT DETECTED | — | NEGATIVE |
| NEG425 | NOT DETECTED | — | NEGATIVE |
| NEG426 | NOT DETECTED | — | NEGATIVE |
| NEG427 | NOT DETECTED | — | NEGATIVE |
| NEG428 | NOT DETECTED | — | NEGATIVE |
| NEG429 | NOT DETECTED | — | NEGATIVE |
| NEG430 | NOT DETECTED | — | NEGATIVE |
| NEG431 | NOT DETECTED | — | NEGATIVE |
| NEG432 | NOT DETECTED | — | NEGATIVE |
| NEG433 | NOT DETECTED | — | NEGATIVE |
| NEG434 | NOT DETECTED | — | NEGATIVE |
| NEG435 | NOT DETECTED | — | NEGATIVE |
| NEG436 | NOT DETECTED | — | NEGATIVE |
| NEG437 | NOT DETECTED | — | NEGATIVE |
| NEG438 | NOT DETECTED | — | NEGATIVE |
| NEG439 | NOT DETECTED | — | NEGATIVE |
| NEG440 | NOT DETECTED | — | NEGATIVE |
| NEG441 | NOT DETECTED | — | NEGATIVE |
| NEG442 | NOT DETECTED | — | NEGATIVE |
| NEG443 | NOT DETECTED | — | NEGATIVE |
| NEG444 | NOT DETECTED | — | NEGATIVE |

### DISCUSSION

The rapid spread of SARS-CoV-2 has challenged the testing capacity of many countries. In the United Kingdom, the testing strategy early on in the outbreak prioritized identifying and protecting essential workers and vulnerable patients and, during the height of the outbreak, only those who were symptomatic were tested. However, to effectively control localized outbreaks as governments begin to ease lockdown policies, a wider testing strategy achieved by decentralised and rapid testing capacity will be of the utmost importance. Currently, reverse transcription, real-time quantitative polymerase chain reaction (RT-qPCR) detection of SARS-CoV-2 RNA remains the gold standard for the diagnosis of COVID-19. Whilst largely reliable and able to evaluate samples through high-throughput processes, RT-qPCR requires trained personnel, RNA extraction, and sophisticated instrumentation, all of which limit the use of RT-qPCR test for decentralized testing. In addition, the rapid rise in the global demand for RT-qPCR testing has resulted in a global shortage in necessary supplies, particularly in kits for RNA extraction.

The invention provides a reverse transcription, loop mediated isothermal amplification (RT-LAMP) assay for the detection of SARS-CoV-2 RNA. The RT-LAMP assay is a colorimetric nucleic acid amplification assay—successful amplification of the target sequence results in a colour change from pink to yellow. The reaction takes place in a single-tube format and simply requires heating at 65° C. for 30 minutes for the reaction to proceed. The RT-LAMP assay is rapid, single-step, and ideal for point-of-care-testing (POCT) as the only equipment required is a heating platform. The assay endpoint can be assessed using a variety of outputs including colour change, fluorescence and absorbance (Becherer, L. et al. 2020). By shortening the sample collection, testing, and readout, the POCT RT-LAMP assay is particularly suited to settings where real-time results would directly impact on patient care, including mobile testing centres, emergency departments, primary care facilities, residential homes, and airports.

Several groups have reported RT-LAMP assays for detecting SARS-CoV-2 RNA (Broughton et al. 2020, Park et al. 2020, Yang et al. 2020, Yu et al. 2020, Yan et al. 2020). The RT-LAMP assay method of the present invention is shown herein to reliably detect 20 copies of an Orf1ab RNA transcript. The method has also been further streamlined as a POCT outside of standard diagnostic laboratories. The formulation has been validated as described herein using several commercial SARS-CoV-2 transcripts, residual oropharyngeal clinical samples and saliva samples from restaurant workers.

Hence, the present invention provides an optimised reverse transcription-loop-mediated isothermal amplification (RT-LAMP) assay for the detection of SARS-CoV-2 from extracted RNA for clinical application. It is shown herein that the stability and reliability of the RT-LAMP assay is improved by the addition of a temperature-dependent switch oligonucleotide to reduce self- or off-target amplification. Also described herein is a freeze-dried master mix for single step RT-LAMP reaction, simplifying the operation for end users and improving long-term storage and transportation. The new RT-LAMP assay has been applied for testing clinical extracted RNA samples extracted from swabs of 72 patients and from saliva of 527 restaurant workers and demonstrated a sensitivity of 95% (95% CI 88.9 to 98.3%) and specificity of 99% (95% CI 97.7% to 99.7%). The assay can detect to <100 copies of SARS-CoV2 RNA. Cross reactivity with other human coronaviruses was not observed. The outcome of RT-LAMP can be reported by both colorimetric detection and quantifiable fluorescent reading. Objective measures with a digitized reading data flow would allow for the sharing of results for local or national surveillance.

The rapid spread of SARS-CoV-2, including to resource limited areas, requests the development of novel, rapid methods for detecting infection in individuals. The standard diagnostic tool, RT-qPCR, cannot be readily deployed outside of large diagnostic laboratories due to the necessary technical expertise, sophisticated instrumentation, and costly reagents required for sample preparation and processing. The RT-LAMP assay is an ideal POCT as the only required equipment is a simple heating platform, which could be a heating block, dry incubator, water bath or thermal cycler. The assay is simple to perform, delivering an accurate result rapidly that can be easily interpreted by several different detection systems including fluorescence and absorbance detection, or simple visual inspection in a POC setting. Effective surveillance depends on the frequency of testing and test turnaround time, which has been shown to be only marginally improved by using a test with a high sensitivity (Larremore et al. 2020).

### A Short Oligo Switch to Minimise False Positives

Several adaptations have been introduced in the present invention to improve on the performance of the RT-LAMP assay. These differentiate the invention from other published RT-LAMP assays for SARS-CoV-2 detection. To minimize the risk of false positive due to unspecific amplification or primer dimers in RT-LAMP (Suleman et al. 2016, Postel et al. 2010, Abbasi et al. 2016), short oligo switch has been introduced with sequence complementary to the FIP primer, which ‘locks’ FIP from random self-amplification in the absence of the target RNA. In the presence of the target RNA sequence of SARS-CoV-2, the switch is competitively inhibited for binding the FIP primer and the RT-LAMP reaction proceeds. The addition of the switch decreased the risk of self- or off-target amplification (FIG. 1) and did not demonstrably impair the sensitivity of the assay (FIG. 2a).

Detection Limit of this RT-LAMP Assay

The RT-LAMP assay of the invention is sensitive and highly specific for SARS-CoV-2 RNA. Neither O117_N nor O117_Q primer set produced false positive results over eight reactions of samples containing the four seasonal coronaviruses. The 50% endpoint values for transcripts 19/304 and RNA control 2 are largely in agreement and demonstrate that the RT-LAMP assay can readily detect <100 copies per reaction of SARS-CoV-2 RNA. The substantially higher 50% endpoint for RNA control 1 may be due to improper initial quantification during shipment or transcript degradation. While a trend towards increased sensitivity in O117_N as one may expect is noted, no statistically significant difference between the O117_N and O117_Q preparations was found when averaged across the 19/304 and RNA Control 2 transcripts, but this may be a consequence of the low sample size.

The RT-LAMP assay demonstrated high concordance with a standard RT-qPCR assay in a laboratory setting. RNA extracts from clinical samples were tested using the RT-LAMP assay and showed reasonably high levels of OPA (90.3%) compared to RT-qPCR. The false negative samples presented have Ct values greater than 31 (Table 1). This suggests that the detection limit of O117_Q is equivalent to Ct=˜31 in an RT-qPCR assay.

The five false positives detected by our RT-LAMP assay from the RNA extract samples raise the concerns of self-amplification and carryover contamination repeatedly observed in RT-LAMP (Suleman et al. 2016, Postel et al. 2010, Abbasi et al. 2016). The present invention can mitigate these risks through the addition of the switch oligonucleotide which demonstrably increases the stability of the reaction. Through freeze-drying the kit components, the process is also simplified for end users and limits the opportunities for onsite carryover contamination.

The application of RT-LAMP has several limitations. Of 16 clinical positive samples with higher Ct values (Ct>31), the assay was able to detect 13 (81%), and thus it may miss identifying some samples with low viral loads. Elution with AVE might affect the RT-LAMP reaction. Therefore the use RNase free water as elution buffer for the rest of RT-LAMP experiments may be beneficial. However, the assay detects the vast majority of samples from patients who are likely to be very infectious and this assay could be used for rapid identification of individuals with medium to high viral loads to divert these samples away from overburdened diagnostic laboratories. Some previous data has reported that a threshold of Ct=33 was threshold for which a patient is no longer considered infectious, as viable virus isolated from patients with a Ct value beyond 33 did not generate a positive culture growth (Walsh et al. 2020, Scola et al. 2020). More recent data, which remains to be confirmed, suggests that infectious virus was not recovered at a Ct above 24 (Binnicker et al. 2020). An important consideration in this discussion is that Ct values are not directly comparable and may differ by as much as 5 cycles when compared directly. It was also found that two sets of primers including O117_Q and S17 can significantly enhance sensitivity (FIG. 10). The switch in O117_Q also prevents false positive results using these two sets of primers.

### Drying Mixes Enables Room Temperature Transport and Storage

Drying the master mix simplifies the process for the end user—all that is required is the addition of water and the patient sample. A further advantage of the dried reagent form is its long-term durability and storage at room temperature. Performance of the dried O117_N and O117_Q kits was not compromised by storage at room temperature for 14 days, although the color of the kits might change to yellow after 5 days due to the change of pH. However, pH and the colour of the kits can be re-adjusted to the original pink colour by adding 2.5 μl of 10 mM KOH. The pH corrected kits still worked as normal in response to water control and full viral RNA transcript (FIG. 3). A fluorescence quantitative reading showed that the dry kits responded well to viral RNA after the dry kits were stored at room temperature for at least 14 days (See FIG. 12), suggesting that Bst 2.0 DNA polymerase and WarmStart reverse transcriptase in NEB mastermix remain active for a long duration under drying storage condition. The dried kits can be transported without cold chain constraints, reducing transport and storage costs, increasing the resilience of the supply chain and providing access to testing for resource limited settings and POCT outside of standard diagnostic laboratories.

### Colorimetric and Fluorescent Double Display to Report RT-LAMP Results

Colorimetric RT-LAMP detection methods rely on the qualitative assessment of a pH-dependent colour change of phenol red as an indirect indication of polymerase amplification of the target sequence. As visual interpretation of the experimental results could be subjective, three different quantitative methods to overcome these limitations have been established. The first method exploits the absorbance changes associated with phenol red as a function of pH and the other two methods utilized fluorescent dyes and functions independent of pH. The fluorescent methods are particularly attractive as they function independent to the pH changes and could potentially overcome the problem associated with buffers present in the sample collection media affecting the readouts associated with phenol red. Although RT-LAMP reactions have been conducted in a qPCR machine with real time fluorescence measurement, this necessitates the use of a qPCR machine. Here we show that these readouts can be performed accurately using relatively inexpensive equipment that is readily available to many laboratories. Furthermore, these methods provide additional flexibility to the end users such that the quantification methods can be chosen according to equipment availability and/or throughput requirements. Where higher throughput is required, the RT-LAMP reaction can be conducted in a multi-well format optimised for 96 or 384 well plates using a fluorescent dye such as SYTO9 and read with a microplate reader. On the other hand, the Qubit 2.0 is a relatively inexpensive desktop fluorometer requiring only a power source and can be readily integrated into the testing workflow in a variety of diverse settings, including universities, schools, GP clinics and airports. The additional advantage of Qubit allows electronic transfer of data which could be linked to local and national surveillance programmes.

Thus, given the high accuracy of our LAMP assay, the potential use of multiple detection systems (including portable options such as Qubit), ease of assay performance and the rapid turn around time, this assay could be used as an effective and highly practical first-line screening tool. This RT-LAMP assay shows high accuracy, acceptable sensitivity, and rapid turnaround time, thereby potentially providing a strategic and affordable way to manage surveillance of the SARS-COV-2 public health crisis.

### Materials And Methods

### Primer Design.

Primers were designed as described above. The primer set O117 targeting Orf1ab of SARS-CoV-2 was designed using PrimerExplorer (http://primerexplorer.jp/e/, Tomita et al. 2008) and each primer was synthesised by Integrated DNA Technologies (IDT, UK). The Switch was synthesised by Integrated DNA Technologies (IDT, UK) with an Iowa Black Dark Quencher at the 3′ end (See Table 1).

10× O117_N primer mix was prepared by mixing equal volumes of 16 μM FIP, 16 μM BIP, 2 μM F3, 2 μM B3, 4 μM LF and 4 μM LB.
10× O117_Q primer mix was prepared by mixing equal volumes of 16 μM FIP, 16 μM BIP, 2 μM F3, 2 μM B3, 4 μM LF, 4 μM LB and 24 μM Switch.

### RT-LAMP Reaction Mix Preparation and Generalized Procedure.

All equipment, laminar-flow cabinets, and working benches were sprayed with 70% w/v ethanol (Sigma-Aldrich Co., UK) and RNaseZap (Sigma-Aldrich Co. UK) prior to reaction mix preparation. All reagents and PCR tubes were kept on ice during kit preparation.

For wet reaction mix, 12.5 μL of WarmStart Colorimetric LAMP 2× Master Mix (DNA & RNA) from New England Biolabs (New England Biolabs, UK), 5 μL of DNase & RNase-free molecular grade water (Thermo Fisher Scientific Ltd), and 2.5 μL of 10× O117_N or 10× O117_Q primer mix was added into PCR tubes and mixed homogeneously.

For dried reaction mix, 12.5 μL 2× Master Mix was supplemented with 7.5 μL drying protective agent, OSD-D1™ (Oxsed Ltd, Oxford) containing sucrose, dextran, lactose and trehalose. 2.5 μL of 10× O117_N or 10× O117_Q primer mix was then added into the PCR tubes. The PCR tubes with caps open were dried with a freeze dryer (VirTis Genesis Pilot Lyophilizer, SP Scientific). After desiccated, importing nitrogen gas to release the vacuum. Seal the caps of the PCR tubes under nitrogen environment. The dried reaction mixes were stored at −20° C. immediately.

The RT-LAMP reaction was run by adding 5 μL of sample of interest to a 20 μL wet reaction mix or a dried reaction mix resuspended in 20 μL of DNase/RNase-free water and heating the reaction at 65° C. for 30 minutes. A positive result was confirmed through a pH-dependent colorimetric change or confirmation of the production of LAMP product using absorbance or fluorescence.

### Full-Length Transcripts.

Three full-length SARS-CoV-2 RNA transcripts were used in this study. Research reagent 19/304 (NIBSC, UK) was the kind gift of Giada Mattiuzo. Synthetic RNA Control 1—MT007544.1 (Twist Bioscience Ltd) and Synthetic RNA Control 2—MN908947.3 (Twist Bioscience Ltd) were the kind gifts of John Taylor (Oxford Medical Genetics Laboratories).

### Clinical Samples.

We confirm that that all methods were carried out in accordance with relevant guidelines and regulations. All experimental protocols were approved by Oxford University. The informed consent was obtained from all subjects or, if subjects are under 18, from a parent and/or legal guardian. Residual samples from clinical testing of 72 symptomatic patients from the Oxford University Hospitals NHS Foundation Trust, Department of Microbiology, were included. The study was registered and accepted as a service evaluation on Oxford University Hospitals Governance System Ulysses register (CSS-MICRO-6330). The samples (527 saliva specimens) from restaurant staff were recruited as part of a testing pilot commissioned by the Food and Environmental Hygiene Department (FEHD), Hong Kong. All restaurant staff consented to taking part in the COVID-19 testing project and were informed that samples would be used for research. Prenetics Ltd were authorised by the Hong Kong government to test restaurant staff (https://www.info.gov/hk/gia/general/202007/17/P2020071700975.htm).

### RT-qPCR

Briefly, for the patient samples oro-nasopharyngeal swabs were stored in Universal Transfer Medium (COPAN Diagnostic, USA) or 0.9% saline and extracted using the QIAsymphony system (Qiagen Co., UK). 200 μL of sample solution was mixed with 430 μL of Buffer OBL and carried into the extraction phase. RNA was eluted into 60 μL of buffer AVE and 10 μL of the elution was carried into a RT-qPCR amplification using the Rotor-Gene Q (Qiagen Co., UK). RT-qPCR reactions were set up using a RNA polymerase gene target validated by Public Health England (PHE, UK) and the Altona RealStar SARS-CoV-2 RT-PCR Kit targeting the E and S genes.

For the restaurant staff deep-throat saliva was taken from 527 participants and diluted with PBS in 1:1 ratio. Diluted DTS of 200 μL was added to the binding buffer of MagMAX Viral/Pathogen Nucleic Acid Isolation Kit (Thermo Fisher Scientific) and viral RNA extraction was carried out according to the manufacturer protocol with the exception that RNA was eluted in 60 μL RNase-free water instead of the elution buffer. The entire isolation process was done in Kingfisher Flex automated system (Thermo Fisher Scientific). RT-PCR for detecting extracted RNA of SARS CoV-2 was carried out according to the US CDC 2019-Novel Coronavirus (2019-nCoV) Real-Time RT-PCR Diagnostic Panel Instructions for Use. RT-PCR targeted N1, N2 of SARS-CoV-2 genes and human RNaseP gene. Briefly, 5 μL of extracted RNA or control, 1.5 μL combined primer/probe mix (IDT, US), 5 μL 4× TaqMan Fast Virus 1-Step Master Mix (Thermo Fisher Scientific) and 8.5 μL RNase-free water were mixed (total volume 20 μL) and loaded into real-time PCR system (Applied Biosciences ViiA7, Thermo Fisher Scientific). The data were analysed using the QuartStudio Real-Time PCR software (Thermo Fisher Scientific).

### RT-LAMP

RT-LAMP for detecting SARS CoV-2 was carried out according to the manufacturing protocol (Oxsed RaViD Direct SARS-CoV-2 Test). For the wet reaction mix, 5 μL of the eluted RNA sample was carried into a reaction containing O117_Q for RT-LAMP assay. For dry reactions, 5 μL of extracted RNA was mixed with 20 μL RNase-free water to reconstitute lyophilized RT-LAMP master mix. The reaction was incubated at 65° C. for 30 min and the colour was recorded.

### Analytic Stability, Sensitivity, and Specificity.

The stability of the wet reaction mix containing O117_N or O117_Q was assessed. 5 μL of 40 copies/μL of Synthetic RNA Control 2 was added as a positive control and 5 μL of 0.2 μg/μL human genome cDNA was added as a negative control. Human genome cDNA was reverse transcribed from whole RNA extracted from human bone marrow derived MSC line (Lonza, UK). RNA extraction was performed as previously described (Brinkhof et al. 2018 and 2006) and reverse transcribed using the QuantiTect Reverse Transcription kit (Qiagen, Manchester, UK). 0.5-1 μg whole RNA was converted into cDNA per manufacturer's instructions including a genomic DNA wipe-out step. Final cDNA sample was diluted to 0.2 μg/μL in RNAse/DNAse free water and stored at −20° C. until used for RT-LAMP assay as negative control. The reaction was run as described above and the colour change was assessed at 0, 30, and 60 minutes.

To obtain an estimate of analytic sensitivity, the 50% endpoint of the LAMP assay was assessed using the three full-length SARS-CoV-2 transcripts. 19/304 was extracted using the viral RNA mini kit (Qiagen Co., UK) and eluted in buffer AVE. Serial dilutions of 5 μL of RNA in buffer AVE were used to spike 20 μL of solution from a 50 μL negative throat swab resuspended in 450 μL of RNase-free water. 25 μL of solution was added to a dried kit containing O117_N or O117_Q primers and were tested in parallel and with five replicates. The LAMP assay was run as described above and assessed via colour change and confirmed via UV-Vis of LAMP product on a 2% agarose gel stained with Sybr-Safe (Thermo Fisher Scientific). The 50% endpoint was calculated using the Reed-Muench method (Reed et al. 1938).

The specificity of the LAMP assay for SARS-CoV-2 RNA against other human-infective coronaviruses was assessed. RNA was extracted from biobanked respiratory samples positive for OC43, HKU1, NL63, and 229E (matched samples were previously collected and processed as described in Gaunt et al. 2010) using the QIAamp Viral RNA Mini Kit (Qiagen Co., UK) according to the manufacturer's instructions. 20 μL of solution from a 50 μL negative throat swab resuspended in 450 μL of RNase-free water was spiked with 5 μL of the RNA eluate. 25 μL was added to a dried kit containing O117_N or O117_Q primers and were tested in parallel. The LAMP assay was run in duplicate at 65° C. for 40 minutes and assessed via colour change.

### Quantitative Detection Methods.

Three methods for converting the qualitative colour change output of RT-LAMP reactions into a quantitative output were assessed. The absorbance spectrum of negative and positive samples was read using a Fluorostar Omega microplate reader (BMG Labtech, UK), and the reaction outcome was assessed using the ratio of 430/560 nm. Detection of the LAMP product was also performed using an intercalating fluorescent dye, SYTO 9, at a final concentration of 3 μM (Thermo Fisher Scientific), added to the reaction mix once the 30 minutes RT-LAMP reaction was completed. Excitation and emission were performed at 485 nm and 500 nm, respectively by Fluorostar Omega microplate reader (BMG Labtech, UK). Alternatively, 5 μL of the reaction product was carried into the Qubit dsDNA BR Assay Kit (Invitrogen, UK). The assay was performed according to the manufacturer's instructions and read using a Qubit 2.0 fluorometer. Cutoff for positive results for absorbance and SYTO 9 quantitative assays were determined to be three times the standard deviations above the negative controls. For Qubit, three times the standard deviation was calculated using negative samples and negative controls.

### Exemplary Primer and Switch Oligonucleotide Sequences

SEQ ID NO: 1 =
GGTTTTCAAGCC
SEQ ID NO: 2 =
GGTTTTCAAGCCAGATTCATTATGGATGTCACAATTCAGAAGTAGGA
SEQ ID NO: 3 =
TCTTCGTAAGGGTGGTCGCAGCACACTTGTTATGGCAAC
SEQ ID NO: 4 =
CCCCAAAATGCTGTTGTT
SEQ ID NO: 5 =
TAGCACGTGGAACCCAAT
SEQ ID NO: 6 =
TCGGCAAGACTATGCTCAGG
SEQ ID NO: 7 =
TTGCCTTTGGAGGCTGTGT
SEQ ID NO: 8 =
GGCTTGAAAACC
SEQ ID NO: 9 =
TCTTTCACACGTGGTGTT
SEQ ID NO: 10 =
GTACCAAAAATCCAGCCTC
SEQ ID NO: 11 =
CATGGAACCAAGTAACATTGGAAAACCTGACAAAGTTTTCAGATCC
SEQ ID NO: 12 =
CTCTGGGACCAATGGTACTAAGAGGACTTCTCAGTGGAAGCA
SEQ ID NO: 13 =
GAAAGGTAAGAACAAGTCCTGAGT
SEQ ID NO: 14 =
CTGTCCTACCATTTAATGATGGTGT

### REFERENCES

Abbasi, I., Kirstein, O. D., Hailu, A. & Warburg, A. Optimization of loop-mediated isothermal amplification (LAMP) assays for the detection of Leishmania DNA in human blood samples. Acta Trop. 162, 20-26 (2016).
Baek, Y. H. et al. Development of a reverse transcription-loop-mediated isothermal amplification as a rapid early-detection method for novel SARS-CoV-2. Emerg. Microbes Infect. 9, 998-1007 (2020).
Becherer, L. et al. Loop-mediated isothermal amplification (LAMP)—review and classification of methods for sequence-specific detection. Anal. Methods 12, 717-746 (2020).
Binnicker, M. J. Can the Severe Acute Respiratory Syndrome Coronavirus 2 Polymerase Chain Reaction Cycle Threshold Value and Time From Symptom Onset to Testing Predict Infectivity?Clin. Infect. Dis. 71, 2667-2668 (2020).
Brinkhof, B. et al. Improving characterisation of human Multipotent Stromal Cells cultured in 2D and 3D: Design and evaluation of primer sets for accurate gene expression normalisation. PLOS ONE 13, e0209772 (2018).
Brinkhof, B., Spee, B., Rothuizen, J. & Penning, L. C. Development and evaluation of canine reference genes for accurate quantification of gene expression. Anal. Biochem. 356, 36-43 (2006).
Broughton, J. P. et al. CRISPR-Cas12-based detection of SARS-CoV-2. Nat. Biotechnol. 1-5 (2020)
Dao Thi, V. L. et al. Screening for SARS-CoV-2 infections with colorimetric RT-LAMP and LAMP sequencing. Preprint at http://medrxiv.org/lookup/doi/10.1101/2020.05.05.20092288 (2020).
Gaunt, E. R., Hardie, A., Claas, E. C. J., Simmonds, P. & Templeton, K. E. Epidemiology and clinical presentations of the four human coronaviruses 229E, HKU1, NL63, and OC43 detected over 3 years using a novel multiplex real-time PCR method. J. Clin. Microbiol. 48, 2940-2947 (2010).
Hsieh, K., Mage, P. L., Csordas, A. T., Eisenstein, M. & Soh, H. T. Simultaneous elimination of carryover contamination and detection of DNA with uracil-DNA-glycosylase-supplemented loop-mediated isothermal amplification (UDG-LAMP). Chem. Commun. 50, 3747-3749 (2014).
Huang et al. RT-LAMP for rapid diagnosis of coronavirus SARS-CoV-2. Microbial Biotechnology 13(5): 950-961 (2020).
Larremore, D. B. et al. Test sensitivity is secondary to frequency and turnaround time for COVID-19 screening. Sci. Adv. November 20:eabd5393 (2020).
Nagamine K, Hase T, Notomi T. Accelerated reaction by loop-mediated isothermal amplification using loop primers. Molecular and Cellular Probes 16, 223-229 (2002).
Niu, P. et al. Three Novel Real-Time RT-PCR Assays for Detection of COVID-19 Virus. China CDC Wkly. 2, 453-457 (2020).
Notomi T, et al. Loop-mediated isothermal amplification of DNA. Nucleic Acids Research 28, (2000).
Park, G.-S. et al. Development of Reverse Transcription Loop-Mediated Isothermal Amplification Assays Targeting Severe Acute Respiratory Syndrome Coronavirus 2 (SARS-CoV-2). J. Mol. Diagn. JMD 22, 729-735 (2020).
Postel, A. et al. Evaluation of two commercial loop-mediated isothermal amplification assays for detection of avian influenza H5 and H7 hemagglutinin genes. J. Vet. Diagn. Investig. Off Publ. Am. Assoc. Vet. Lab. Diagn. Inc 22, 61-66 (2010).
Reed, L. J. & Muench, H. A SIMPLE METHOD OF ESTIMATING FIFTY PERCENT ENDPOINTS. Am. J. Epidemiol. 27, 493-497 (1938).
Scola, L. & Al, B. et. Viral RNA load as determined by cell culture as a management tool for discharge of SARS-CoV-2 patients from infectious disease wards: draft. (2020).
Suleman, E., Mtshali, M. S. & Lane, E. Investigation of false positives associated with loop-mediated isothermal amplification assays for detection of Toxoplasma gondii in archived tissue samples of captive felids. J. Vet. Diagn. Investig. Off Publ. Am. Assoc. Vet. Lab. Diagn. Inc 28, 536-542 (2016).
Tanner, N. A., Zhang, Y. & Evans, T. C. Visual detection of isothermal nucleic acid amplification using pH-sensitive dyes. BioTechniques 58, 59-68 (2015).
Thi, V. L. D. et al. A colorimetric RT-LAMP assay and LAMP-sequencing for detecting SARS-CoV-2 RNA in clinical samples. Sci. Transl. Med. 12, (2020).
Tomita, N., Mori, Y., Kanda, H. & Notomi, T. Loop-mediated isothermal amplification (LAMP) of gene sequences and simple visual detection of products. Nat. Protoc. 3, 877-882 (2008).
Walsh, K. A. et al. SARS-CoV-2 detection, viral load and infectivity over the course of an infection. J. Infect. 81, 357-371 (2020).
Yan, C. et al. Rapid and visual detection of 2019 novel coronavirus (SARS-CoV-2) by a reverse transcription loop-mediated isothermal amplification assay. Clin Microbiol Infect 26, 773-779 (2020).
Yang, W. et al. Rapid Detection of SARS-CoV-2 Using Reverse transcription RT-LAMP method. medRxiv 2020.03.02.20030130 (2020) doi:10.1101/2020.03.02.20030130.
Yu, L. et al. Rapid Detection of COVID-19 Coronavirus Using a Reverse Transcriptional Loop-Mediated Isothermal Amplification (RT-LAMP) Diagnostic Platform. Clin. Chem. 66, 975-977 (2020).

1. A set of oligonucleotide primers for use in a method of amplifying a fragment of DNA, the set of primers comprising a forward inner primer (FIP), a reverse inner primer (BIP), a forward outer primer (F3), a reverse outer primer (B3), and a switch oligonucleotide, wherein the switch oligonucleotide comprises a nucleotide sequence that is complementary to a fragment of one of the forward or reverse primers, wherein the switch oligonucleotide is adapted to anneal to the complementary primer at temperatures that are below the temperature range for amplification of the DNA, and wherein the switch oligonucleotide prevents amplification from the complementary primer when the complementary primer is bound to the switch oligonucleotide.

2. A method of reducing false positives in the detection of a target DNA or RNA sequence using loop-mediated isothermal amplification (LAMP) or reverse transcription loop-mediated isothermal amplification (RT-LAMP), wherein the method comprises including a switch oligonucleotide in the LAMP or RT-LAMP reaction, wherein the switch oligonucleotide comprises a nucleotide sequence that is complementary to a fragment of a forward inner (FIP), reverse inner (BIP), forward outer (F3) or reverse outer (B3) primer used for the amplification, wherein the switch oligonucleotide is adapted to anneal to the complementary primer at temperatures that are below the temperature range for the amplification, and wherein the switch oligonucleotide prevents amplification from the complementary primer when the complementary primer is bound to the switch oligonucleotide.

3. A method of loop-mediated isothermal amplification (LAMP) or reverse transcription loop-mediated isothermal amplification (RT-LAMP), the method comprising
(a) mixing the set of primers of claim 1 with template DNA or RNA, deoxyribonucleotide triphosphates (dNTP), DNA polymerase and optionally reverse transcriptase in solution; and
(b) heating the mixture to the working temperature of the DNA polymerase.

4. The set of primers of claim 1 or the method of claim 2 or claim 3, wherein the switch oligonucleotide is adapted to prevent elongation at the 3′ end of the switch oligonucleotide.

5. The set of primers or method of claim 4, wherein the switch oligonucleotide comprises a dark quencher moiety, optionally wherein one or more of the primers comprises a fluorophore.

6. The set of primers or method of any one of claims 1 to 5, wherein the switch oligonucleotide is complementary to the forward loop primer or the reverse loop primer.

7. The set of primers or method of any one of claims 1 to 6, wherein the set of primers further comprises a forward loop primer (LF) and/or a reverse loop primer (LB).

8. A kit for amplifying a fragment of DNA, wherein the kit comprises the set of primers of any one of claims 1 and 4 to 7.

9. The kit of claim 7, which further comprises:
(i) DNA polymerase;
(ii) reverse transcriptase;
(iii) a pH indicator and/or colorimetric indicator;
(iv) a fluorophore;
(v) deoxyribonucleotide triphosphates (dNTP);
(vi) buffer components; and/or
(vii) instructions for use.

10. The kit of claim 8 or claim 9, wherein the primers and optionally one or more of the additional components of the kit are dried and optionally combined as a reagent mix.

11. Use of the set of primers of any one of claims 1 and 4 to 7 or the kit of any one of claims 8 to 10 in a method of detecting or amplifying a target DNA or RNA sequence.

12. The set of primers of any one of claims 1 and 4 to 7, the kit of any one of claim 8 to 10, or the use of claim 11, wherein the method comprises reverse transcription of an RNA to produce cDNA and amplification of the reverse transcribed cDNA.

13. The set of primers, method, kit or use of any one of claims 1 to 12, wherein the switch oligonucleotide is in excess of the complementary primer.

14. The set of primers, method, kit or use of any one of claims 1 to 13, wherein the method is for detecting a polynucleotide of a pathogen in a sample.

15. The set of primers, method, kit or use of claim 14, wherein the pathogen is a Coronaviridae.

16. The set of primers, method, kit or use of claim 16, wherein the Coronaviridae is SARS-CoV-2.

17. The set of primers, method, kit or use of claim 15 or 16, wherein the complementary primer comprises the nucleotide sequence of SEQ ID NO: 1 or SEQ ID NO: 2, or a variant having at least 50% sequence identity to SEQ ID NO: 1 or SEQ ID NO: 2, and/or wherein the switch oligonucleotide comprises the nucleotide sequence of SEQ ID NO: 8 or a variant having at least 50% sequence identity to SEQ ID NO: 8.

18. The set of primers, method, kit or use of claim 17, wherein the set of primers further includes a reverse inner primer (BIP) comprising the nucleotide sequence of SEQ ID NO: 3 or a variant having at least 50% sequence identity to SEQ ID NO: 3; a forward outer primer (F3) comprising the nucleotide sequence of SEQ ID NO: 4 or a variant having at least 50% sequence identity to SEQ ID NO: 4; a reverse outer primer (B3) comprising the nucleotide sequence of SEQ ID NO: 5 or a variant having at least 50% sequence identity to SEQ ID NO: 5; a forward loop primer (LF) comprising the nucleotide sequence of SEQ ID NO: 6 or a variant having at least 50% sequence identity to SEQ ID NO: 6; and/or a reverse loop primer (BF) comprising the nucleotide sequence of SEQ ID NO: 7 or a variant having at least 50% sequence identity to SEQ ID NO: 7.

19. The set of primers, method, kit or use of claim 18, wherein the set of primers comprise six further primers having the nucleotide sequences of SEQ ID NOS: 9 to 14.

20. A kit for detecting SARS-CoV-2 or for diagnosing a SARS-CoV-2 infection or Covid-19 in a subject, the kit comprising a set of six primers and a switch oligonucleotide having the nucleotide sequences of SEQ ID NOs: 2 to 8 or a set of 12 primers and a switch oligonucleotide having the nucleotide sequences of SEQ ID NOs: 2 to 14, a DNA polymerase, a reverse transcriptase, a colorimetric pH indicator, deoxyribonucleotide triphosphates (dNTP), and optionally a buffer, optionally wherein the kit comprises a vacuum-dried reagent mix.

21. A method of detecting SARS-CoV-2 in a sample or diagnosing a SARS-CoV-2 infection or COVID-19 in a subject, the method comprising
(i) obtaining the sample, or obtaining a biological sample from the subject;
(ii) reverse transcription to produce cDNA from RNA in the sample;
(iii) amplification of the reverse transcribed cDNA using a set of six primers and a switch oligonucleotide having the nucleotide sequences of SEQ ID NOs: 2 to 8 or a set of 12 primers and a switch oligonucleotide having the nucleotide sequences of SEQ ID NOs: 2 to 14;
(iv) detecting the amplified DNA; and
determining the presence of SARS-CoV-2 or diagnosing a SARS-CoV-2 infection or COVID-19 in the subject.


SEQUENCE LISTING (14 sequences)

SEQ ID NO: 1 - DNA, 12 bp, Artificial Sequence
  primer
ggttttcaagcc

SEQ ID NO: 2 - DNA, 47 bp, Artificial Sequence
  primer
ggttttcaagccagattcattatggatgtcacaattcagaagtagga

SEQ ID NO: 3 - DNA, 39 bp, Artificial Sequence
  primer
tcttcgtaagggtggtcgcagcacacttgttatggcaac

SEQ ID NO: 4 - DNA, 18 bp, Artificial Sequence
  primer
ccccaaaatgctgttgtt

SEQ ID NO: 5 - DNA, 18 bp, Artificial Sequence
  primer
tagcacgtggaacccaat

SEQ ID NO: 6 - DNA, 20 bp, Artificial Sequence
  primer
tcggcaagactatgctcagg

SEQ ID NO: 7 - DNA, 19 bp, Artificial Sequence
  primer
ttgcctttggaggctgtgt

SEQ ID NO: 8 - DNA, 12 bp, Artificial Sequence
  switch oligonucleotide
ggcttgaaaacc

SEQ ID NO: 9 - DNA, 18 bp, Artificial Sequence
  primer
tctttcacacgtggtgtt

SEQ ID NO: 10 - DNA, 19 bp, Artificial Sequence
  primer
gtaccaaaaatccagcctc

SEQ ID NO: 11 - DNA, 46 bp, Artificial Sequence
  primer
catggaaccaagtaacattggaaaacctgacaaagttttcagatcc

SEQ ID NO: 12 - DNA, 42 bp, Artificial Sequence
  primer
ctctgggaccaatggtactaagaggacttctcagtggaagca

SEQ ID NO: 13 - DNA, 24 bp, Artificial Sequence
  primer
gaaaggtaagaacaagtcctgagt

SEQ ID NO: 14 - DNA, 25 bp, Artificial Sequence
  primer
ctgtcctaccatttaatgatggtgt